(19) United States
(12) Patent Application Publication
(10) Pub. No.: US 2026/0209790 A1
(43) Pub. Date: Jul. 23, 2026

(54) MODIFIED INTERNAL RIBOSOME ENTRY SITE (IRES) ELEMENTS AND METHODS OF USE

(71) Applicant: Gilead Sciences, Inc., Foster City, CA (US)

(72) Inventor: Bo Jiang, Vista, CA (US)

(21) Appl. No.: 19/395,512

(22) Filed: Nov. 20, 2025

Related U.S. Application Data
(60) Provisional application No. 63/724,727, filed on Nov. 25, 2024.

(51) Int. Cl. C12N 15/85; C07K 14/52; C07K 16/00

(52) U.S. Cl. CPC C12N 15/85; C07K 14/52; C07K 16/00; C07K 2317/31; C07K 2317/55; C07K 2317/569; C07K 2319/30; C12N 2830/50; C12N 2840/203

(57) ABSTRACT

Provided are improved IRES variants for improved expression of a protein of interest in a mammalian host cell.

### CROSS-REFERENCE TO RELATED APPLICATIONS

This application claims the benefit under 35 U.S.C. § 119(e) of U.S. Provisional Application No. 63/724,727, filed on Nov. 25, 2024, which is hereby incorporated herein by reference in its entirety for all purposes.

### SEQUENCE LISTING

The instant application contains a Sequence Listing which has been submitted electronically in .XML format and is hereby incorporated by reference in its entirety. Said .XML copy, created on Sep. 26, 2025, is named 1457-US-WO_SL.xml and is 67,258 bytes in size.

### BACKGROUND

The goal of cell line development (CLD) within the biotech and biopharmaceutical industry is to generate clonal-derived, high-level producing, and stable cell lines suitable for clinical and commercial manufacturing of desired therapeutic proteins (e.g., a monoclonal antibody, a multispecific binding molecule, an Fc fusion protein or an immunoadhesin). Typically, the CLD process involves 1) construction of the expression vector encoding the desired therapeutic protein (i.e., having a gene-of-interest (GOI) encoding a protein of interest (POI)); 2) introducing the expression vector into a expression host (most often Chinese Hamster Ovary (CHO)) cells via transfection; 3) selecting for cells in which the GOI-containing expression vector has been stably integrated into the host genome; 4) screening and identifying clonal cell lines that express high levels of the protein of interest with desirable product qualities and long-term sustainable productivity suitable to large-scale clinical and commercial manufacturing processes. After entering host cells, the expression vector DNA molecules integrate into the host genome randomly, both in terms of the integration location and the number of vector copies to be integrated. Consequently, the expression levels of the GOI can vary tremendously among different cells post transfection, with most of the stably transfected cells exhibiting low expression levels of the protein of interest, because most of the integration locations withing the host genome do not support high levels of recombinant gene expression. In order to identify clonal cell lines that express high levels of the protein of interest suitable for clinical and commercial manufacturing, a very large number (e.g., usually in the range of 1000s) of transfected cell pools and single-cell derived clonal cell lines have to be screened and characterized. This makes the CLD a very labor-intensive and time-consuming process.

Several strategies have been adopted by the biotechnology and biopharmaceutical industry to improve the efficiency and/or shorten the timeline of the CLD process. Implementation of automated liquid handling capacity has dramatically increased the throughput and streamlined the pool/clone screening workflow. However, automation alone has little impact in reducing the overall CLD timeline. Targeted-integration methodology has been used to introduce the GOI expression cassette into pre-defined genomic locus (e.g., an expression “hot-spot”), which nearly eliminated the need to screen large numbers of pools/clones and significantly shortened the overall CLD timeline. However, the targeted-integration approach requires the identification of expression hot-spot(s) within the host genome. In addition, the productivity of cell lines obtained from targeted integration generally will be lower than those obtained from random integration, mainly due to the lower copy number of the expression vector integrated by targeted-integration technology.

### SUMMARY

In one aspect, provided is a modified, non-naturally occurring internal ribosome entry site (IRES) comprising a polynucleotide sequence having at least 95%, 96%, 97%, 98% or 99% sequence identity, to SEQ ID NO: 1, and comprising one or both of: (i) 1-5 modifications (e.g., substitutions, additions or deletions) within positions 135-155, e.g., within positions 136-150 (e.g., within stem loop H depicted in FIG. 1); and/or (ii) 1-5 modifications (e.g., substitutions, additions or deletions) within positions 265-280, e.g., within positions 269-274 (e.g., within hammerhead I depicted in FIG. 1), wherein the positions are with reference to SEQ ID NO: 1. In some embodiments, the modified IRES comprises a polynucleotide sequence of any one of SEQ ID NOs: 3-4, 9-25 and 33-42 or a polynucleotide sequence comprising at least 95%, 96%, 97%, 98% or 99% sequence identity to any one of SEQ ID NOs: 3-4, 9-25 and 33-42. In some embodiments, the modified IRES is no longer than 557 nucleotide bases in length.

In another aspect, provided is an expression cassette comprising a modified IRES comprising a polynucleotide sequence of any one of SEQ ID NOs: 3-4, 9-25 and 33-42. In some embodiments, the expression cassette comprises from 5′ to 3′ the following operably linked elements: (1) A promoter; (2) A polynucleotide encoding a protein of interest; (3) A modified IRES comprising a polynucleotide sequence of any one of SEQ ID NOs: 3-4, 9-25 and 33-42, e.g., of any one of SEQ ID NOs: 12-14, 17, 22, 24, 40 and 42; (4) A selection marker, e.g., a polynucleotide encoding a glutamine synthetase (GS); and (5) A polyadenylation (polyA) signal sequence. In some embodiments, the protein of interest is a therapeutic protein. In some embodiments, the therapeutic protein is selected from an antibody and an immunoadhesin (e.g., an Fc fusion protein). In some embodiments, the antibody is a multi-specific antibody. In some embodiments, the promoter is a constitutive promoter. In some embodiments, the promoter promotes or induces expression of the protein of interest in a mammalian host cell. In some embodiments, the promoter is selected from a CMV promoter, a CAG promoter, an EF1a promoter, a PGK promoter, a SV40 promoter, an UBC promoter, and a beta-actin promoter. In some embodiments, the modified IRES comprises a polynucleotide sequence of any one of SEQ ID NOs: 12-14, 17, 22, 24, 40 and 42. In some embodiments, the selection marker comprises a polynucleotide encoding a glutamine synthetase (GS) or an antibiotic resistance protein. In some embodiments, the antibiotic resistance protein is encoded by NeoR, BsdR, HygR, PuroR, BleoR (a.k.a., ZeoR). In some embodiments, the GS is selected from a human GS, a fish GS, a koala GS, a hamster GS and variants thereof. In some embodiments, the GS is encoded by a polynucleotide of any one of SEQ ID NOs: 26-30. In some embodiments, the GS is a human GS or a koala GS. In some embodiments, the modified IRES comprises SEQ ID NO: 40, and the selection marker comprises a polynucleotide encoding a human GS or a hamster GS. In some embodiments, the modified IRES comprises SEQ ID NO: 40, and the selection marker comprises a polynucleotide of SEQ ID NO: 28 or SEQ ID NO: 29. In some embodiments, the GS is encoded by a polynucleotide of any one of SEQ ID NOs: 26 and 29-30. In some embodiments, the modified IRES is selected from SEQ ID NOs: 11-25 and 42, and the selection marker comprises a polynucleotide encoding human variant GS-R324S (SEQ ID NO: 30). In some embodiments, the modified IRES is selected from SEQ ID NOs: 12-14, 22, 24 and 42, and the selection marker comprises a polynucleotide encoding human variant GS-R324S (SEQ ID NO: 30). In some embodiments, the modified IRES is selected from SEQ ID NOs: 12-14 and 42, and the selection marker comprises a polynucleotide encoding human variant GS R324S (SEQ ID NO: 30). In some embodiments, the modified IRES is selected from SEQ ID NOs: 11-25 and 42, and the selection marker comprises a polynucleotide encoding an antibiotic resistance gene, e.g., BleoR. In some embodiments, the modified IRES is selected from SEQ ID NOs: 12-14, 16, 17, 22 and 24, and the selection marker comprises a polynucleotide encoding an antibiotic resistance gene, e.g., BleoR. In some embodiments, the modified IRES is selected from SEQ ID NOs: 16, 17, 22 and 24, and the selection marker comprises a polynucleotide encoding an antibiotic resistance gene, e.g., BleoR. In some embodiments, the modified IRES comprises SEQ ID NO: 17, and the selection marker comprises a polynucleotide encoding an antibiotic resistance gene, e.g., BsdR or ZeoR. In some embodiments, the polyA signal sequence is a SV40 polyA or a bovine growth hormone (BGH) polyA.

In a further aspect, provided is an expression cassette comprising from 5′ to 3′ the following operably linked elements: (1) A promoter; (2) A polynucleotide encoding an Fc fusion protein or an immunoadhesin; (3) A modified IRES comprising a polynucleotide of any one of SEQ ID NOs: 3-4, 9-25 and 33-42; (4) A selection marker encoding a human GS or a koala GS; and (5) A polyadenylation (polyA) signal sequence. In some embodiments, the modified IRES comprises a polynucleotide sequence of any one of SEQ ID NOs: 12-14, 17, 22, 24, 40 and 42. In some embodiments, the modified IRES is selected from SEQ ID NOs: 11-25 and 42, and the selection marker comprises a polynucleotide encoding human variant GS-R324S (SEQ ID NO: 30). In some embodiments, the modified IRES is selected from SEQ ID NOs: 12-14, 22, 24 and 42, and the selection marker comprises a polynucleotide encoding human variant GS-R324S (SEQ ID NO: 30). In some embodiments, the modified IRES is selected from SEQ ID NOs: 12-14 and 42, and the selection marker comprises a polynucleotide encoding human variant GS R324S (SEQ ID NO: 30). In some embodiments, the modified IRES is selected from SEQ ID NOs: 11-25 and 42, and the selection marker comprises a polynucleotide encoding an antibiotic resistance gene, e.g., BleoR. In some embodiments, the modified IRES is selected from SEQ ID NOs: 12-14, 16, 17, 22 and 24, and the selection marker comprises a polynucleotide encoding an antibiotic resistance gene, e.g., BleoR. In some embodiments, the modified IRES is selected from SEQ ID NOs: 16, 17, 22 and 24, and the selection marker comprises a polynucleotide encoding an antibiotic resistance gene, e.g., BleoR. In some embodiments, the polyA signal sequence is a SV40 polyA or a bovine growth hormone (BGH) polyA.

In a further aspect, provided is a vector comprising one or more expression cassettes as described above and herein. In some embodiments, the vector comprises two or more expression cassettes as described above and herein. In some embodiments, the vector comprises three or more expression cassettes as described above and herein. In some embodiments, the vector comprises four or more expression cassettes as described above and herein. In some embodiments, the vector is a plasmid. In some embodiments, the vector is a mammalian expression vector. In some embodiments, the vector integrates into the genome of a host cell.

In a further aspect, provided is a vector comprising a set of two or more expression cassettes, at least one expression cassette comprising a modified IRES comprising a polynucleotide sequence of any one of SEQ ID NOs: 3-4, 9-25 and 33-42. In some embodiments, the vector comprises a set of expression cassettes comprising from 5′ to 3′ the following operably linked elements: (1) A first promoter; (2) A first polynucleotide encoding a first polypeptide of the protein of interest; (3) A first polyA signal sequence; (4) A second promoter; (5) A second polynucleotide encoding a second polypeptide of the protein of interest; (6) A modified IRES comprising a polynucleotide sequence of any one of SEQ ID NOs: 3-4, 9-25 and 33-42; (7) A selection marker; and (8) A second polyA signal sequence. In some embodiments, the vector comprises a set of expression cassettes comprising from 5′ to 3′ the following operably linked elements: (1) A first promoter; (2) A first polynucleotide encoding a first polypeptide of the protein of interest; (3) A modified IRES comprising a polynucleotide sequence of any one of SEQ ID NOs: 3-4, 9-25 and 33-42; (4) A selection marker; (5) A first polyA signal sequence; (6) A second promoter; (7) A second polynucleotide encoding a second polypeptide of the protein of interest; and (8) A second polyA signal sequence. In some embodiments, the vector comprises a set of expression cassettes comprising from 5′ to 3′ the following operably linked elements: (1) A first promoter; (2) A first polynucleotide encoding a first polypeptide of the protein of interest; (3) A first modified IRES comprising a polynucleotide sequence of any one of SEQ ID NOs: 3-4, 9-25 and 33-42; (4) A first selection marker; (5) A first polyA signal sequence; (6) A second promoter; (7) A second polynucleotide encoding a second polypeptide of the protein of interest; (8) A second modified IRES, optionally comprising a polynucleotide sequence of any one of SEQ ID NOs: 3-4, 9-25 and 33-42; (9) A second selection marker, optionally, wherein the second selection marker is different from the first selection marker; (10) A second polyA signal sequence. In some embodiments, the vector comprises a set of expression cassettes comprising from 5′ to 3′ the following operably linked elements: (1) A first promoter; (2) A first polynucleotide encoding an antibody light chain; (3) A first polyA signal sequence; (4) A second promoter; (5) A second polynucleotide encoding an antibody heavy chain; (6) A modified IRES comprising a polynucleotide sequence of any one of SEQ ID NOs: 3-4, 9-25 and 33-42; (7) A selection marker; and (8) A second polyA signal sequence. In some embodiments, the vector comprises a set of expression cassettes comprising from 5′ to 3′ the following operably linked elements: (1) A first promoter; (2) A first polynucleotide encoding an antibody heavy chain; (3) A first polyA signal sequence; (4) A second promoter; (5) A second polynucleotide encoding an antibody light chain; (6) A modified IRES comprising a polynucleotide sequence of any one of SEQ ID NOs: 3-4, 9-25 and 33-42; (7) A selection marker, optionally, wherein the second selection marker is different from the first selection marker; and (8) A second polyA signal sequence. In some embodiments, the vector comprises a set of expression cassettes comprising from 5′ to 3′ the following operably linked elements: (1) A first promoter; (2) A first polynucleotide encoding an antibody light chain; (3) A modified IRES comprising a polynucleotide sequence of any one of SEQ ID NOs: 3-4, 9-25 and 33-42; (4) A selection marker; (5) A first polyA signal sequence; (6) A second promoter; (7) A second polynucleotide encoding an antibody heavy chain; and (8) A second polyA signal sequence. In some embodiments, the vector comprises a set of expression cassettes comprising from 5′ to 3′ the following operably linked elements: (1) A first promoter; (2) A first polynucleotide encoding an antibody heavy chain; (3) A modified IRES comprising a polynucleotide sequence of any one of SEQ ID NOs: 3-4, 9-25 and 33-42; (4) A selection marker; (5) A first polyA signal sequence; (6) A second promoter; (7) A second polynucleotide encoding an antibody light chain; and (8) A second polyA signal sequence. In some embodiments, the vector comprises a set of expression cassettes comprising from 5′ to 3′ the following operably linked elements: (1) A first promoter; (2) A first polynucleotide encoding an antibody light chain; (3) A first modified IRES comprising a polynucleotide sequence of any one of SEQ ID NOs: 3-4, 9-25 and 33-42; (4) A first selection marker; (5) A first polyA signal sequence; (6) A second promoter; (7) A second polynucleotide encoding an antibody heavy chain; (8) A second modified IRES, optionally comprising a polynucleotide sequence of any one of SEQ ID NOs: 3-4, 9-25 and 33-42; (9) A second selection marker, optionally, wherein the second selection marker is different from the first selection marker; and (10) A second polyA signal sequence. In some embodiments, the vector comprises a set of expression cassettes comprising from 5′ to 3′ the following operably linked elements: (1) A first promoter; (2) A first polynucleotide encoding an antibody heavy chain; (3) A first modified IRES comprising a polynucleotide sequence of any one of SEQ ID NOs: 3-4, 9-25 and 33-42; (4) A first selection marker; (5) A first polyA signal sequence; (6) A second promoter; (7) A second polynucleotide encoding an antibody light chain; (8) A second modified IRES, optionally comprising a polynucleotide sequence of any one of SEQ ID NOs: 3-4, 9-25 and 33-42; (9) A second selection marker, optionally, wherein the second selection marker is different from the first selection marker; and (10) A second polyA signal sequence. In some embodiments, the vector comprises a set of expression cassettes comprising from 5′ to 3′ the following operably linked elements: (1) A first promoter; (2) A first polynucleotide encoding a first polypeptide of the protein of interest; (3) A first polyA signal sequence; (4) A second promoter; (5) A second polynucleotide encoding a second polypeptide of the protein of interest; (6) A second polyA signal sequence; (7) A third promoter; (8) A third polynucleotide encoding a third polypeptide of the protein of interest; (9) A modified IRES as described herein, e.g., of any one of SEQ ID NOs: 3-4, 9-25 and 33-42, e.g., of any one of SEQ ID NOs: 12-14, 17, 22, 24, 40 and 42; (10) A selection marker, e.g., a polynucleotide encoding a GS; and (11) A third polyA signal sequence. In some embodiments, the vector comprises a set of expression cassettes comprising from 5′ to 3′ the following operably linked elements: (1) A first promoter; (2) A first polynucleotide encoding a first polypeptide of the protein of interest; (3) A modified IRES as described herein, e.g., of any one of SEQ ID NOs: 3-4, 9-25 and 33-42, e.g., of any one of SEQ ID NOs: 12-14, 17, 22, 24, 40 and 42; (4) A selection marker, e.g., a polynucleotide encoding a GS; (5) A first polyA signal sequence; (6) A second promoter; (7) A second polynucleotide encoding a second polypeptide of the protein of interest; (8) A second polyA signal sequence; (9) A third promoter; (10) A third polynucleotide encoding a third polypeptide of the protein of interest; and (11) A third polyA signal sequence. In some embodiments, the vector comprises a set of expression cassettes comprising from 5′ to 3′ the following operably linked elements: (1) A first promoter; (2) A first polynucleotide encoding a first polypeptide of the protein of interest; (3) A first polyA signal sequence; (4) A second promoter; (5) A second polynucleotide encoding a second polypeptide of the protein of interest; (6) A modified IRES as described herein, e.g., of any one of SEQ ID NOs: 3-4, 9-25 and 33-42, e.g., of any one of SEQ ID NOs: 12-14, 17, 22, 24, 40 and 42; (7) A selection marker, e.g., a polynucleotide encoding a GS; (8) A second polyA signal sequence; (9) A third promoter; (10) A third polynucleotide encoding a third polypeptide of the protein of interest; and (11) A third polyA signal sequence. In some embodiments, the vector comprises a set of expression cassettes comprising from 5′ to 3′ the following operably linked elements: (1) A first promoter; (2) A first polynucleotide encoding a first polypeptide of the protein of interest; (3) A first modified IRES as described herein, e.g., of any one of SEQ ID NOs: 3-4, 9-25 and 33-42, e.g., of any one of SEQ ID NOs: 12-14, 17, 22, 24, 40 and 42; (4) A first selection marker, e.g., a polynucleotide encoding a GS; (5) A first polyA signal sequence; (6) A second promoter; (7) A second polynucleotide encoding a second polypeptide of the protein of interest; (8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42; (9) A second selection marker, optionally, wherein the second selection marker is different from the first selection marker; (10) A second polyA signal sequence; (11) A third promoter; (12) A third polynucleotide encoding a third polypeptide of the protein of interest; (13) A third modified IRES, optionally comprising a polynucleotide of any one of SEQ ID NOs: 3-4, 9-25 and 33-42, e.g., of any one of SEQ ID NOs: 12-14, 17, 22, 24, 40 and 42; (14) A third selection marker, optionally wherein the third selection marker is different from the first selection marker and the second selection marker; and (15) A third polyA signal sequence. In some embodiments, the vector comprises a set of expression cassettes comprising from 5′ to 3′ the following operably linked elements: (1) A first promoter; (2) A first polynucleotide encoding an antibody light chain; (3) A first polyA signal sequence; (4) A second promoter; (5) A second polynucleotide encoding an antibody heavy chain; (6) A modified IRES as described herein, e.g., of any one of SEQ ID NOs: 3-4, 9-25 and 33-42, e.g., of any one of SEQ ID NOs: 12-14, 17, 22, 24, 40 and 42; (7) A selection marker, e.g., a polynucleotide encoding a GS; (8) A second polyA signal sequence; (9) A third promoter; (10) A third polynucleotide encoding an Fc fusion protein or a scFv-Fc fusion protein; and (11) A third polyA signal sequence. In some embodiments, the vector comprises a set of expression cassettes comprising from 5′ to 3′ the following operably linked elements: (1) A first promoter; (2) A first polynucleotide encoding an antibody light chain; (3) A first polyA signal sequence; (4) A second promoter; (5) A second polynucleotide encoding an antibody heavy chain; (6) A second polyA signal sequence; (7) A third promoter; (8) A third polynucleotide encoding an Fc fusion protein or a scFv-Fc fusion protein; (9) A modified IRES as described herein, e.g., of any one of SEQ ID NOs: 3-4, 9-25 and 33-42, e.g., of any one of SEQ ID NOs: 12-14, 17, 22, 24, 40 and 42; (10) A selection marker, e.g., a polynucleotide encoding a GS; and (11) A third polyA signal sequence. In some embodiments, the vector comprises a set of expression cassettes comprising from 5′ to 3′ the following operably linked elements: (1) A first promoter; (2) A first polynucleotide encoding an antibody light chain; (3) A first polyA signal sequence; (4) A second promoter; (5) A second polynucleotide encoding an antibody heavy chain; (6) A first modified IRES as described herein, e.g., of any one of SEQ ID NOs: 3-4, 9-25 and 33-42, e.g., of any one of SEQ ID NOs: 12-14, 17, 22, 24, 40 and 42; (7) A first selection marker, e.g., a polynucleotide encoding a GS; (8) A second polyA signal sequence; (9) A third promoter; (10) A third polynucleotide encoding an Fc fusion protein or a scFv-Fc fusion protein; (11) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42; (12) A second selection marker, optionally, wherein the second selection marker is different from the first selection marker; and (13) A third polyA signal sequence. In some embodiments, the vector comprises a set of expression cassettes comprising from 5′ to 3′ the following operably linked elements: (1) A first promoter; (2) A first polynucleotide encoding an antibody heavy chain; (3) A first polyA signal sequence; (4) A second promoter; (5) A second polynucleotide encoding an antibody light chain; (6) A modified IRES as described herein, e.g., of any one of SEQ ID NOs: 3-4, 9-25 and 33-42, e.g., of any one of SEQ ID NOs: 12-14, 17, 22, 24, 40 and 42; (7) A selection marker, e.g., a polynucleotide encoding a GS; (8) A second polyA signal sequence; (9) A third promoter; (10) A third polynucleotide encoding an Fe fusion protein or a scFv-Fc fusion protein; and (11) A third polyA signal sequence. In some embodiments, the vector comprises a set of expression cassettes comprising from 5′ to 3′ the following operably linked elements: (1) A first promoter; (2) A first polynucleotide encoding an antibody heavy chain; (3) A first polyA signal sequence; (4) A second promoter; (5) A second polynucleotide encoding an antibody light chain; (6) A second polyA signal sequence; (7) A third promoter; (8) A third polynucleotide encoding an Fc fusion protein or a scFv-Fc fusion protein; (9) A modified IRES as described herein, e.g., of any one of SEQ ID NOs: 3-4, 9-25 and 33-42, e.g., of any one of SEQ ID NOs: 12-14, 17, 22, 24, 40 and 42; (10) A selection marker, e.g., a polynucleotide encoding a GS; and (11) A third polyA signal sequence. In some embodiments, the vector comprises a set of expression cassettes comprising from 5′ to 3′ the following operably linked elements: (1) A first promoter; (2) A first polynucleotide encoding an antibody heavy chain; (3) A first polyA signal sequence; (4) A second promoter; (5) A second polynucleotide encoding an antibody light chain; (6) A first modified IRES as described herein, e.g., of any one of SEQ ID NOs: 3-4, 9-25 and 33-42, e.g., of any one of SEQ ID NOs: 12-14, 17, 22, 24, 40 and 42; (7) A selection marker, e.g., a polynucleotide encoding a GS; (8) A second polyA signal sequence; (9) A third promoter; (10) A third polynucleotide encoding an Fc fusion protein or a scFv-Fc fusion protein; (11) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42; (12) A selection marker, e.g., a polynucleotide encoding a GS; and (13) A third polyA signal sequence. In some embodiments, the vector comprises a set of expression cassettes comprising from 5′ to 3′ the following operably linked elements: (1) A first promoter; (2) A first polynucleotide encoding an antibody light chain; (3) A modified IRES as described herein, e.g., of any one of SEQ ID NOs: 3-4, 9-25 and 33-42, e.g., of any one of SEQ ID NOs: 12-14, 17, 22, 24, 40 and 42; (4) A selection marker, e.g., a polynucleotide encoding a GS; (5) A first polyA signal sequence; (6) A second promoter; (7) A second polynucleotide encoding an antibody heavy chain; (8) A second polyA signal sequence; (9) A third promoter; (10) A third polynucleotide encoding an Fc fusion protein or a scFv-Fc fusion protein; and (11) A third polyA signal sequence. In some embodiments, the vector comprises a set of expression cassettes comprising from 5′ to 3′ the following operably linked elements: (1) A first promoter; (2) A first polynucleotide encoding an antibody heavy chain; (3) A modified IRES as described herein, e.g., of any one of SEQ ID NOs: 3-4, 9-25 and 33-42, e.g., of any one of SEQ ID NOs: 12-14, 17, 22, 24, 40 and 42; (4) A selection marker, e.g., a polynucleotide encoding a GS; (5) A first polyA signal sequence; (6) A second promoter; (7) A second polynucleotide encoding an antibody light chain; (8) A second polyA signal sequence; (9) A third promoter; (10) A third polynucleotide encoding an Fc fusion protein or a scFv-Fc fusion protein; and (11) A third polyA signal sequence. In some embodiments, the vector comprises a set of expression cassettes comprising from 5′ to 3′ the following operably linked elements: (1) A first promoter; (2) A first polynucleotide encoding an antibody light chain; (3) A first modified IRES as described herein, e.g., of any one of SEQ ID NOs: 3-4, 9-25 and 33-42, e.g., of any one of SEQ ID NOs: 12-14, 17, 22, 24, 40 and 42; (4) A first selection marker, e.g., a polynucleotide encoding a GS; (5) A first polyA signal sequence; (6) A second promoter; (7) A second polynucleotide encoding an antibody heavy chain; (8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42; (9) A second selection marker, optionally, wherein the second selection marker is different from the first selection marker; (10) A second polyA signal sequence; (11) A third promoter; (12) A third polynucleotide encoding an Fc fusion protein or a scFv-Fc fusion protein; (13) A third modified IRES, optionally comprising a polynucleotide of any one of SEQ ID NOs: 3-4, 9-25 and 33-42, e.g., of any one of SEQ ID NOs: 12-14, 17, 22, 24, 40 and 42; (14) A third selection marker, optionally wherein the third selection marker is different from the first selection marker and the second selection marker; and (15) A third polyA signal sequence. In some embodiments, the vector comprises a set of expression cassettes comprising from 5′ to 3′ the following operably linked elements: (1) A first promoter; (2) A first polynucleotide encoding an antibody heavy chain; (3) A first modified IRES as described herein, e.g., of any one of SEQ ID NOs: 3-4, 9-25 and 33-42, e.g., of any one of SEQ ID NOs: 12-14, 17, 22, 24, 40 and 42; (4) A first selection marker, e.g., a polynucleotide encoding a GS; (5) A first polyA signal sequence; (6) A second promoter; (7) A second polynucleotide encoding an antibody light chain; (8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42; (9) A second selection marker, optionally, wherein the second selection marker is different from the first selection marker; (10) A second polyA signal sequence; (11) A third promoter; (12) A third polynucleotide encoding an Fc fusion protein or a scFv-Fc fusion protein; (13) A third modified IRES, optionally comprising a polynucleotide of any one of SEQ ID NOs: 3-4, 9-25 and 33-42, e.g., of any one of SEQ ID NOs: 12-14, 17, 22, 24, 40 and 42; (14) A third selection marker, optionally wherein the third selection marker is different from the first selection marker and the second selection marker; and (15) A third polyA signal sequence.

With respect to further embodiments of the vectors described herein, in some embodiments, the first promoter and the second promoter are selected from selected from a CMV promoter, a CAG promoter, an EF1a promoter, a PGK promoter, a SV40 promoter, an UBC promoter, and a beta-actin promoter. In some embodiments, the first promoter and the second promoter are the same. In some embodiments, the first promoter and the second promoter are different. In some embodiments, the modified IRES comprises a polynucleotide sequence of any one of SEQ ID NOs: 12-14, 17, 22, 24, 40 and 42. In some embodiments, the first modified IRES and the second modified IRES are the same. In some embodiments, the first modified IRES and the second modified IRES are different. In some embodiments, the selection marker is a polynucleotide encoding a glutamine synthetase (GS) or an antibiotic resistance protein. In some embodiments, the first selection marker comprises a polynucleotide encoding a GS and the second selection marker comprises a polynucleotide encoding an antibiotic resistance gene. In some embodiments, the first selection marker comprises a polynucleotide encoding an antibiotic resistance gene and the second selection marker comprises a polynucleotide encoding a GS. In some embodiments, the antibiotic resistance protein is encoded by NeoR, BsdR, HygR, PuroR, BleoR (a.k.a., ZeoR). In some embodiments, the GS is a human GS, a fish GS, a koala GS, a hamster GS and variants thereof. In some embodiments, the GS is encoded by a polynucleotide of any one of SEQ ID NOs: 26-30. In some embodiments, the GS is a human GS or a koala GS. In some embodiments, the GS is encoded by a polynucleotide of any one of SEQ ID NOs: 26 and 29-30. In some embodiments, the modified IRES is selected from SEQ ID NOs: 11-25 and 42, and the first selection marker comprises human variant GS-R324S (SEQ ID NO: 30). In some embodiments, the modified IRES is selected from SEQ ID NOs: 12-14, 22, 24 and 42, and the selection marker comprises human variant GS-R324S (SEQ ID NO: 30). In some embodiments, the modified IRES is selected from SEQ ID NOs: 12-14 and 42, and the selection marker comprises human variant GS R324S (SEQ ID NO: 30). In some embodiments, the polyA signal sequence is a SV40 polyA signal sequence or a bovine growth hormone (BGH) polyA signal sequence. In some embodiments, the vector is a plasmid. In some embodiments, the vector is a mammalian expression vector. In some embodiments, the vector integrates into the genome of a host cell.

In a further aspect, provided are host cells comprising one or more of the vectors described herein, comprising one or more of the vectors described herein, comprising one or more of the modified IRES comprising a polynucleotide sequence of any one of SEQ ID NOs: 3-4, 9-25 and 33-42, described herein. In some embodiments, the host cell is a mammalian cell. In some embodiments, the mammalian cell is a Chinese hamster ovary (CHO) cell. In some embodiments, the host cell comprises a first vector and a second vector; (a) the first vector comprising an expression cassette comprising from 5′ to 3′ the following operably linked elements: (1) A first promoter; (2) A polynucleotide encoding an antibody heavy chain; (3) A first modified IRES comprising a polynucleotide of any one of SEQ ID NOs: 3-4, 9-25 and 33-42; (4) A first selection marker; and (5) A polyadenylation (polyA) signal sequence; and (b) the second vector comprising a set of expression cassettes comprising from 5′ to 3′ the following operably linked elements: (1) A second promoter; (2) A polynucleotide encoding an antibody light chain; (3) A second polyA signal sequence; (4) A second modified IRES, optionally comprising a polynucleotide sequence of any one of SEQ ID NOs: 3-4, 9-25 and 33-42; (5) A second selection marker, optionally, wherein the second selection marker is different from the first selection marker; and (6) A second polyA signal sequence. In some embodiments, the host cell comprises a first vector and a second vector: (a) the first vector comprising an expression cassette comprising from 5′ to 3′ the following operably linked elements: (1) A first promoter; (2) A polynucleotide encoding an Fc fusion protein or an immunoadhesin; (3) A first modified IRES comprising a polynucleotide of any one of SEQ ID NOs: 3-4, 9-25 and 33-42; (4) A first selection marker; and (5) A polyadenylation (polyA) signal sequence; and (b) the second vector comprising a set of expression cassettes comprising from 5′ to 3′ the following operably linked elements: (1) A second promoter; (2) A polynucleotide encoding an antibody light chain; (3) A second polyA signal sequence; (4) A third promoter; (5) A polynucleotide encoding an antibody heavy chain; (6) A second modified IRES, optionally comprising a polynucleotide sequence of any one of SEQ ID NOs: 3-4, 9-25 and 33-42; (7) A second selection marker, optionally, wherein the second selection marker is different from the first selection marker; and (8) A third polyA signal sequence. In some embodiments, the host cell comprises a first vector and a second vector: (a) the first vector comprising an expression cassette comprising from 5′ to 3′ the following operably linked elements: (1) A first promoter; (2) A polynucleotide encoding an Fc fusion protein or an immunoadhesin; (3) A first modified IRES comprising a polynucleotide of any one of SEQ ID NOs: 3-4, 9-25 and 33-42; (4) A first selection marker; and (5) A polyadenylation (polyA) signal sequence; and (b) the second vector comprising a set of expression cassettes comprising from 5′ to 3′ the following operably linked elements: (1) A second promoter; (2) A polynucleotide encoding an antibody heavy chain; (3) A second polyA signal sequence; (4) A third promoter; (5) A polynucleotide encoding an antibody light chain; (6) A second modified IRES, optionally comprising a polynucleotide sequence of any one of SEQ ID NOs: 3-4, 9-25 and 33-42; (7) A second selection marker, optionally, wherein the second selection marker is different from the first selection marker; and (8) A third polyA signal sequence. In some embodiments, the host cell comprises a first vector and a second vector: (a) the first vector comprising an expression cassette comprising from 5′ to 3′ the following operably linked elements: (1) A first promoter; (2) A polynucleotide encoding an Fe fusion protein or an immunoadhesin; (3) A first modified IRES comprising a polynucleotide of any one of SEQ ID NOs: 3-4, 9-25 and 33-42; (4) A first selection marker; and (5) A polyadenylation (polyA) signal sequence; and (b) the second vector comprising a set of expression cassettes comprising from 5′ to 3′ the following operably linked elements: (1) A second promoter; (2) A polynucleotide encoding an antibody light chain; (3) A second modified IRES, optionally comprising a polynucleotide sequence of any one of SEQ ID NOs: 3-4, 9-25 and 33-42; (4) A second selection marker, optionally, wherein the second selection marker is different from the first selection marker; (5) A second polyA signal sequence; (6) A third promoter; (7) A polynucleotide encoding an antibody heavy chain; and (8) A third polyA signal sequence. In some embodiments, the host cell comprises a first vector and a second vector: (a) the first vector comprising an expression cassette comprising from 5′ to 3′ the following operably linked elements: (1) A first promoter; (2) A polynucleotide encoding an Fc fusion protein or an immunoadhesin; (3) A first modified IRES comprising a polynucleotide of any one of SEQ ID NOs: 3-4, 9-25 and 33-42; (4) A first selection marker; and (5) A polyadenylation (polyA) signal sequence; and (b) the second vector comprising a set of expression cassettes comprising from 5′ to 3′ the following operably linked elements: (1) A second promoter; (2) A polynucleotide encoding an antibody heavy chain; (3) A second modified IRES, optionally comprising a polynucleotide sequence of any one of SEQ ID NOs: 3-4, 9-25 and 33-42; (4) A second selection marker, optionally, wherein the second selection marker is different from the first selection marker; (5) A second polyA signal sequence; (6) A third promoter; (7) A polynucleotide encoding an antibody light chain; and (8) A third polyA signal sequence. In some embodiments, the host cell comprises a first vector and a second vector: (a) the first vector comprising an expression cassette comprising from 5′ to 3′ the following operably linked elements: (1) A first promoter; (2) A polynucleotide encoding an Fc fusion protein or an immunoadhesin; (3) A first modified IRES comprising a polynucleotide of any one of SEQ ID NOs: 3-4, 9-25 and 33-42; (4) A first selection marker; and (5) A polyadenylation (polyA) signal sequence; and (b) the second vector comprising a set of expression cassettes comprising from 5′ to 3′ the following operably linked elements: (1) A second promoter; (2) A polynucleotide encoding an antibody light chain; (3) A second modified IRES, optionally comprising a polynucleotide sequence of any one of SEQ ID NOs: 3-4, 9-25 and 33-42; (4) A second selection marker, optionally, wherein the second selection marker is different from the first selection marker; (5) A second polyA signal sequence; (6) A third promoter; (7) A polynucleotide encoding an antibody heavy chain; (8) A third modified IRES, optionally comprising a polynucleotide sequence of any one of SEQ ID NOs: 3-4, 9-25 and 33-42; (9) A third selection marker; and (10) A third polyA signal sequence. In some embodiments, the host cell comprises a first vector and a second vector: (a) the first vector comprising an expression cassette comprising from 5′ to 3′ the following operably linked elements: (1) A first promoter; (2) A polynucleotide encoding an Fc fusion protein or an immunoadhesin; (3) A first modified IRES comprising a polynucleotide of any one of SEQ ID NOs: 3-4, 9-25 and 33-42; (4) A first selection marker; and (5) A polyadenylation (polyA) signal sequence; and (b) the second vector comprising a set of expression cassettes comprising from 5′ to 3′ the following operably linked elements: (1) A second promoter; (2) A polynucleotide encoding an antibody heavy chain; (3) A second modified IRES, optionally comprising a polynucleotide sequence of any one of SEQ ID NOs: 3-4, 9-25 and 33-42; (4) A second selection marker, optionally, wherein the second selection marker is different from the first selection marker; (5) A second polyA signal sequence; (6) A third promoter; (7) A polynucleotide encoding an antibody light chain; (8) A third modified IRES, optionally comprising a polynucleotide sequence of any one of SEQ ID NOs: 3-4, 9-25 and 33-42; (9) A third selection marker; and (10) A third polyA signal sequence.

In a further aspect, provided are methods of expressing a protein of interest, comprising: (a) Introducing into a mammalian host cell a vector as described herein; and (b) Culturing the host cell under conditions to allow expression of the protein of interest. In some embodiments, the mammalian host cell is a CHO cell. In some embodiments, at least 1 g/L e.g., at least 1.2 g/L, at least 1.4 g/L, at least 1.6 g/L, at least 1.8 g/L, at least 2.0 g/L, at least 3.0 g/L, at least 4.0 g/L, at least 5.0 g/L, at least 6.0 g/L, at least 7.0 g/L, at least 8.0 g/L, at least 9.0 g/L, of the protein of interest is expressed. In some embodiments, at least 2-fold, e.g., at least 5-fold, at least 10-fold, at least 15-fold, at least 20-fold, of the protein of interest is expressed in comparison to a host cell comprising an expression cassette comprising a wild-type encephalomyocarditis virus (EMCV) IRES277 (SEQ ID NO:1).

### BRIEF DESCRIPTION OF THE DRAWINGS

FIGS. 1A-1F illustrate RNA secondary structure of wild-type encephalomyocarditis virus (EMCV) IRES RNA sequence is GenBank Accession number M81861.1 (DNA sequence provided as SEQ ID NO: 32). Adopted from Duke, et al., J Virol (1992) 66(3):1602-9.

FIG. 2 illustrates a schematic of an expression cassette comprising an IRES.

FIG. 3 illustrates modified IRES elements increased GS-selection stringency. Expression vector format in the 5′ to 3′ direction: CMV promoter (CMVp)-green fluorescent protein (GFP)-internal ribosome entry site (IRES)-glutamine synthase (GS)-SV40 polyadenylation (polyA) signal sequence. ires277 denotes the wild-type, un-modified IRES element (corresponding to nucleotides 277-834 of GenBank entry M81861.1). All other IRES variants described herein are variants of ires277, having the specified nucleotide modification(s). The variant IRES sequences enabled effective death/elimination of cells that produced no or low levels of GS, as indicated by the much lower cell viabilities observed during the early stage of the selection period, and a much delayed active-growth (recovery) phase post-transfection.

FIGS. 4A-4B. FIG. 4A illustrates that the modified IRES sequences increased cell death during post-transfection stable pool selection. FIG. 4B illustrates that the modified IRES sequences increased stable pool recovery time post-transfection. Expression vector format in the 5′ to 3′ direction: CMVp-GFP-IRES-GS-SV40 polyA signal sequence.

FIG. 5 illustrates that the modified IRES sequences increased GFP expression in stably transfected cell pools. Expression vector format in the 5′ to 3′ direction: CMVp-GFP-IRES-GS-SV40 polyA signal sequence.

FIG. 6 illustrates that modified IRES elements resulted in substantially increased (bulk pool) expression levels of an Fc fusion protein (Fc-1), which is encoded by a single open reading frame (ORF). Expression vector format in the 5′ to 3′ direction: CMVp-Fc fusion protein-IRES-GS-SV40 polyA signal sequence.

FIG. 7 illustrates that modified IRES elements resulted in elevated (bulk pool) expression levels of a therapeutic monoclonal antibody (mAb-1), which is encoded by two open reading frames (ORFs) (heavy chain and light chain). Expression vector format for the modified IRES-containing vectors in the 5′ to 3′ direction: CMVp-LC-BGH polyA signal sequence-CMVp-HC-IRES-GS-SV40 polyA signal sequence. Expression vector format for non-IRES vector in the 5′ to 3′ direction: CMVp-LC-BGH polyA signal sequence-CMVp-HC-BGH polyA signal sequence-SV40p-GS-SV40 polyA signal sequence.

FIG. 8 illustrates that combining IRES-variant with non-hamster GS gene further improved expression levels for monoclonal antibody mAb-1. Expression vector format for the modified IRES-containing vectors in the 5′ to 3′ direction: CMVp-LC-BGH polyA signal sequence-CMVp-HC-IRES-GS-SV40 polyA signal sequence.

FIG. 9 illustrates that IRES variants described herein paired with human GS R324S mutant improved expression levels for monoclonal antibody mAb-2. Expression vector format for the modified IRES-containing vectors in the 5′ to 3′ direction: CMVp-LC-IRES-GS-BGH polyA signal sequence-CMVp-HC-SV40 polyA signal sequence. Expression vector format for non-IRES vector in the 5′ to 3′ direction: CMVp-LC-BGH polyA signal sequence-CMVp-HC-BGH polyA signal sequence-SV40p-GS-SV40 polyA signal sequence.

FIG. 10 illustrates that IRES variant described herein paired with human GS R324S mutant improved expression levels for Fc fusion protein Fc-1. Expression vector format for the modified IRES-containing vectors in the 5′ to 3′ direction: CMVp-Fc-IRES-GS-SV40 polyA signal sequence. Expression vector format for non-IRES vector in the 5′ to 3′ direction: CMVp-Fc-BGH polyA signal sequence-SV40p-GS-SV40 polyA signal sequence.

FIGS. 11A-B depict the fed-batch expression levels of a targeted cytokine observed from bulk pools transfected with vectors containing different IRES variants and human GS-R324S gene. Expression vector format for the modified IRES-containing vectors in the 5′ to 3′ direction: CMVp-LC1 CDS-IRES (modified variant)-human GS-R324S CDS-BGH (bovine growth hormone) polyA signal. Expression vector format for non-IRES vectors in the 5′ to 3′ direction: CMVp-HC1 CDS-BGH polyA signal-SV40p-BSD (blasticidin resistence gene) CDS-SV40 polyA signal, and CMVp-HC2 CDS-BGH polyA signal-SV40p-BSD (blasticidin resistence gene) CDS-SV40 polyA signal.

FIG. 12 illustrates fed-batch Fc-1 expression levels observed from bulk pools transfected with vectors containing different IRES variants and the ZeoR gene as the selectable marker.

FIG. 13 illustrates fed-batch bsAb-1 expression levels observed from bulk pools transfected with vectors containing different IRES variants and the hamster or the wild-type human GS gene as the selectable marker.

FIG. 14 illustrates fed-batch VHH1-VHH2-Fc-1 expression levels observed from bulk pools transfected with vectors containing different IRES variants and the hamster or the wild-type human GS gene as the selectable marker.

### DETAILED DESCRIPTION

### 1. Introduction

Provided are improved internal ribosome entry site (IRES) variants based on a truncated sequence of wild-type encephalomyocarditis virus (EMCV) IRES RNA sequence (GenBank Accession number M81861.1; DNA sequence provided as SEQ ID NO: 32). The herein described IRES variants find use for increased expression of biologic molecules, e.g., having one polypeptide chain (e.g., Fc fusion proteins or immunoadhesins), two polypeptide chains (e.g., an immunoglobulin heavy chain (Hc) and an immunoglobulin light chain (Lc), e.g., such as monoclonal antibodies), three polypeptide chains (e.g., asymmetric bispecific molecules having Hc, Lc and a third polypeptide, e.g., an Fc fusion protein, a cytokine Fc fusion protein, a receptor extracellular domain Fc fusion protein, an scFv-Fc fusion protein) or four polypeptide chains (e.g., Fab-Fab format bispecific molecules). Illustrative variant IRES polynucleotide sequences are provided in Table A.

### 2. Variant IRESs

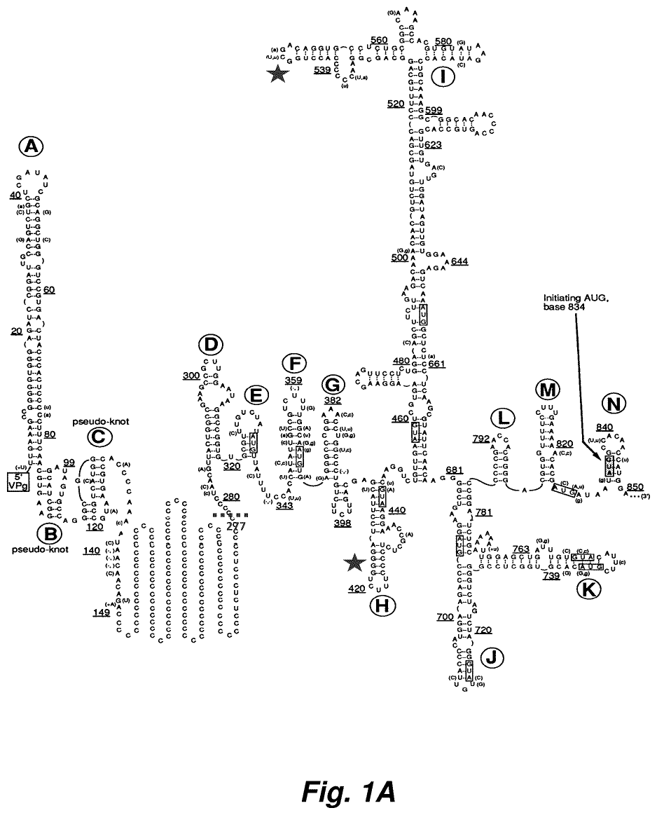

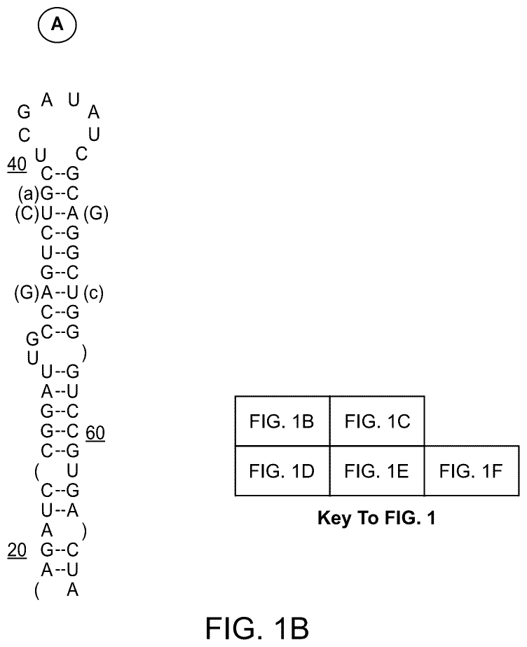

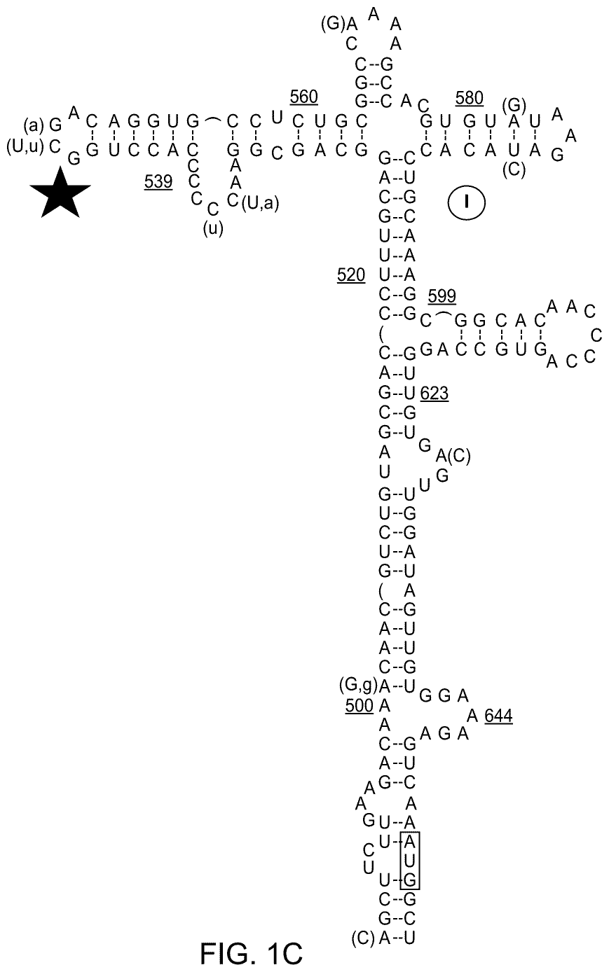

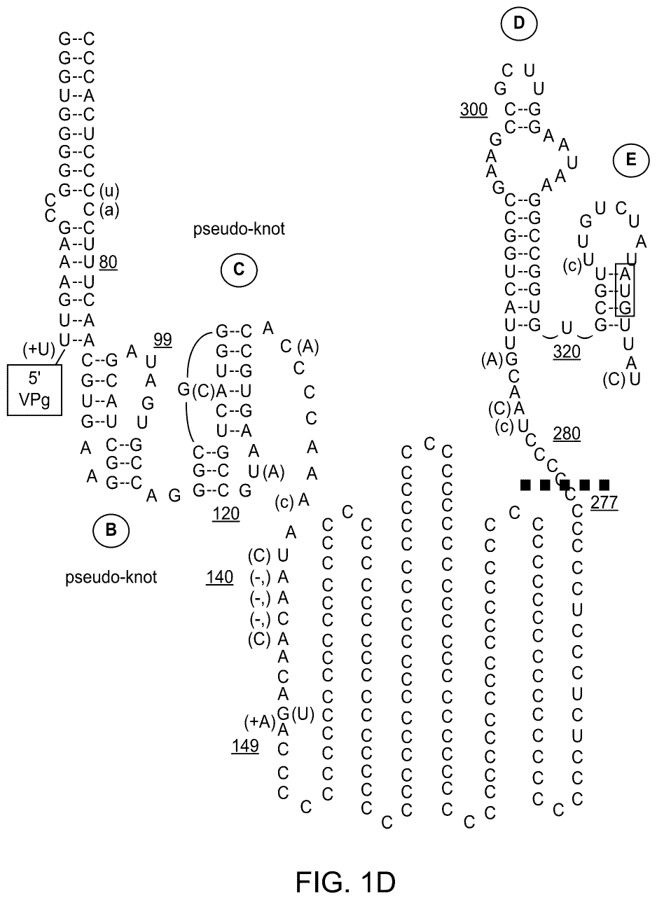

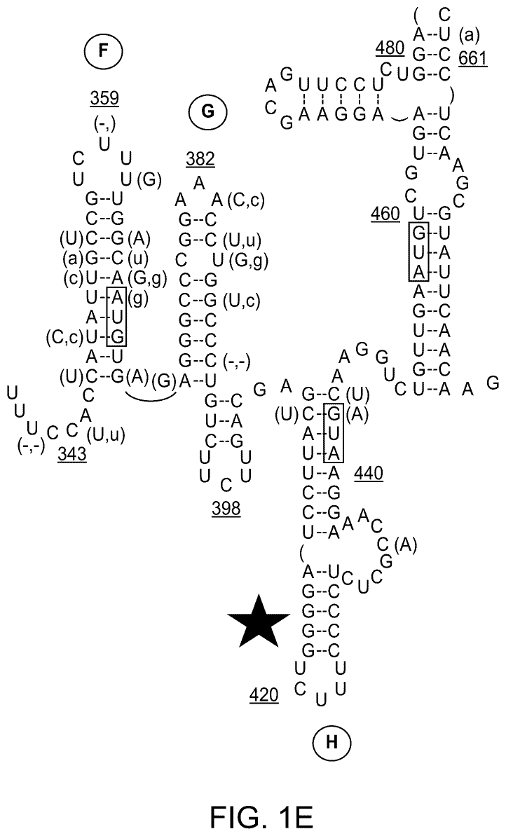

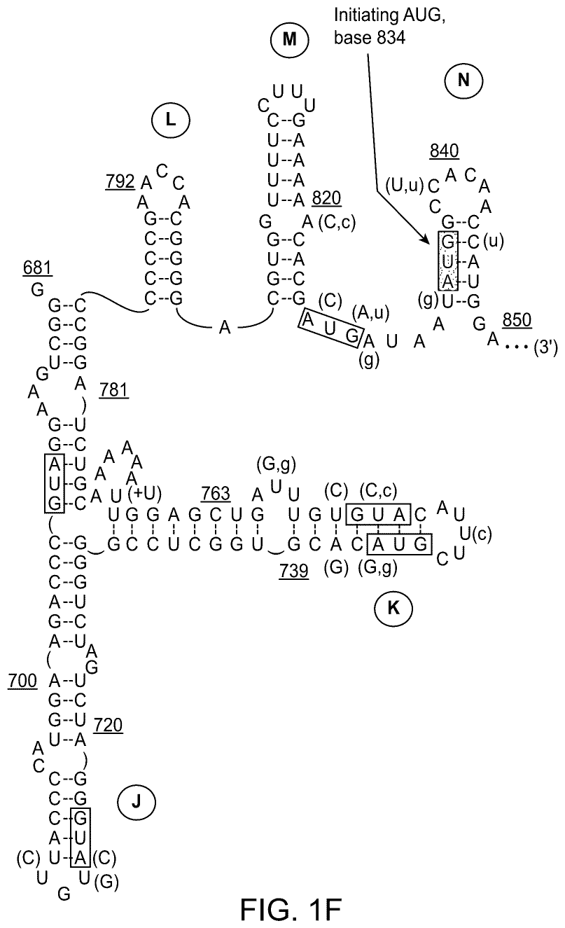

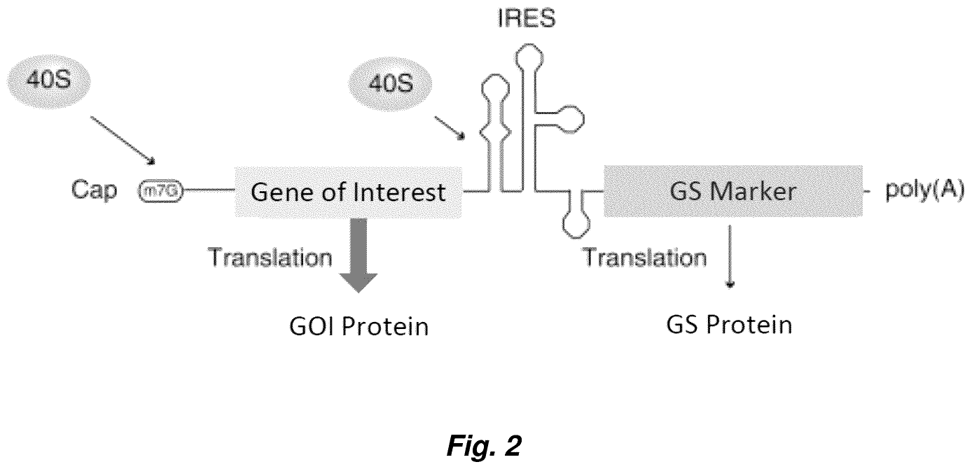

Generally, the variant IRES sequences described herein are based on a truncated form of wild-type encephalomyocarditis virus (EMCV) (GenBank Accession number M81861.1), where the first 276 nucleic acid bases are truncated and nucleic acid bases corresponding to positions 277-835 are retained (hereinafter referred to as “ires277”; SEQ ID NO:1 and depicted in FIG. 1). An “internal ribosome entry site” or “IRES” refers to an RNA element that binds to the 40S ribosomal subunit and allows for translation initiation in a 5′-cap-independent manner.

Generally, the herein described variant IRES sequences are modified (e.g., have one or more substitutions, additions or deletions) within one or both of positions 135-155, e.g., within positions 136-150 and positions 265-280, e.g., within positions 269-274, wherein the positions are with reference to SEQ ID NO: 1. A polynucleotide “variant,” as the term is used herein, is a polynucleotide that typically differs from a polynucleotide specifically disclosed herein in one or more substitutions, deletions, additions and insertions. Such variants may be naturally occurring or may be synthetically generated, for example, by modifying one or more of the polynucleotide sequences described herein and evaluating one or more biological activities of the encoded polypeptide as described herein, e.g., using any of a number of techniques well known in the art.

In various embodiments, the modified IRES comprises a polynucleotide sequence having at least 95%, 96%, 97%, 98% or 99% sequence identity, to SEQ ID NO: 1, and comprises from 1-5, e.g., from 1-4, e.g., from 1-3, e.g., from 1-2 modifications (e.g., substitutions, additions or deletions) or 1 modification, wherein the modifications are within positions 135-155, e.g., within positions 136-150 (e.g., within stem loop H depicted in FIG. 1), wherein the positions are with reference to SEQ ID NO: 1. In various embodiments, the modified IRES comprises a polynucleotide sequence having at least 95%, 96%, 97%, 98% or 99% sequence identity, to SEQ ID NO: 1, and comprises from 1-5, e.g., from 1-4, e.g., from 1-3, e.g., from 1-2 modifications (e.g., substitutions, additions or deletions) or 1 modification, wherein the modifications are within positions 265-280, e.g., within positions 269-274 (e.g., within hammerhead I depicted in FIG. 1), wherein the positions are with reference to SEQ ID NO: 1. In various embodiments, the modified IRES comprises a polynucleotide sequence having at least 95%, 96%, 97%, 98% or 99% sequence identity, to SEQ ID NO: 1, comprises from 1-5, e.g., from 1-4, e.g., from 1-3, e.g., from 1-2 modifications (e.g., substitutions, additions or deletions) or 1 modification, wherein the modifications are within positions 135-155, e.g., within positions 136-150 (e.g., within stem loop H depicted in FIG. 1), and comprises from 1-5, e.g., from 1-4, e.g., from 1-3, e.g., from 1-2 modifications (e.g., substitutions, additions or deletions) or 1 modification, wherein the modifications are within positions 265-280, e.g., within positions 269-274 (e.g., within hammerhead I depicted in FIG. 1), wherein the positions are with reference to SEQ ID NO: 1. In some embodiments, the foregoing genera exclude SEQ ID NOs. 2 and 5-8. As used herein “sequence identity” is determined by aligning a polynucleotide of interest to a reference sequence using an appropriate alignment algorithm, set to default parameters. One example of algorithms that are suitable for determining percent sequence identity and sequence similarity are the BLAST and BLAST 2.0 algorithms, which are described in Altschul et al. (1977) Nucl. Acids Res.25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides described herein. Software for performing BLAST analyses is publicly available through the National Center for Biotechnology Information (blast.ncbi.nlm.nih.gov/Blast.cgi). As appropriate, sequence alignments can be determined along the full length of the polynucleotide sequence of interest.

Illustrative variant IRES sequences modified within positions 135-155, e.g., within positions 136-150, wherein the positions are with reference to SEQ ID NO: 1, include SEQ ID NOs: 1-4, 9-13 and 19-21. Illustrative variant IRES sequences modified within positions 265-280, e.g., within positions 269-274, wherein the positions are with reference to SEQ ID NO: 1, include SEQ ID NOs: 5-8 and 14-18. Illustrative variant IRES sequences modified both within positions 135-155, e.g., within positions 136-150, e.g., within positions 146-149, and within positions 265-280, e.g., within positions 269-274, e.g., within positions 270-272, wherein the positions are with reference to SEQ ID NO: 1, include SEQ ID NOs: 22-25 and 33-42.

In some embodiments, the modified internal ribosome entry site (IRES) comprises a polynucleotide sequence of any one of SEQ ID NOs: 3-4, 9-25 and 33-42. In some embodiments, the modified IRES comprises a polynucleotide sequence of SEQ ID NO: 3, or a polynucleotide having at least 95%, 96%, 97%, 98% or 99% sequence identity to SEQ ID NO: 3. In some embodiments, the modified IRES comprises a polynucleotide sequence of SEQ ID NO: 4, or a polynucleotide having at least 95%, 96%, 97%, 98% or 99% sequence identity to SEQ ID NO: 4. In some embodiments, the modified IRES comprises a polynucleotide sequence of SEQ ID NO: 9, or a polynucleotide having at least 95%, 96%, 97%, 98% or 99% sequence identity to SEQ ID NO: 9. In some embodiments, the modified IRES comprises a polynucleotide sequence of SEQ ID NO: 10, or a polynucleotide having at least 95%, 96%, 97%, 98% or 99% sequence identity to SEQ ID NO: 10. In some embodiments, the modified IRES comprises a polynucleotide sequence of SEQ ID NO: 11, or a polynucleotide having at least 95%, 96%, 97%, 98% or 99% sequence identity to SEQ ID NO: 11. In some embodiments, the modified IRES comprises a polynucleotide sequence of SEQ ID NO: 12, or a polynucleotide having at least 95%, 96%, 97%, 98% or 99% sequence identity to SEQ ID NO: 12. In some embodiments, the modified IRES comprises a polynucleotide sequence of SEQ ID NO: 13, or a polynucleotide having at least 95%, 96%, 97%, 98% or 99% sequence identity to SEQ ID NO: 13. In some embodiments, the modified IRES comprises a polynucleotide sequence of SEQ ID NO: 14, or a polynucleotide having at least 95%, 96%, 97%, 98% or 99% sequence identity to SEQ ID NO: 14. In some embodiments, the modified IRES comprises a polynucleotide sequence of SEQ ID NO: 15, or a polynucleotide having at least 95%, 96%, 97%, 98% or 99% sequence identity to SEQ ID NO: 15. In some embodiments, the modified IRES comprises a polynucleotide sequence of SEQ ID NO: 16, or a polynucleotide having at least 95%, 96%, 97%, 98% or 99% sequence identity to SEQ ID NO: 16. In some embodiments, the modified IRES comprises a polynucleotide sequence of SEQ ID NO: 17, or a polynucleotide having at least 95%, 96%, 97%, 98% or 99% sequence identity to SEQ ID NO: 17. In some embodiments, the modified IRES comprises a polynucleotide sequence of SEQ ID NO: 18, or a polynucleotide having at least 95%, 96%, 97%, 98% or 99% sequence identity to SEQ ID NO: 18. In some embodiments, the modified IRES comprises a polynucleotide sequence of SEQ ID NO: 19, or a polynucleotide having at least 95%, 96%, 97%, 98% or 99% sequence identity to SEQ ID NO: 19. In some embodiments, the modified IRES comprises a polynucleotide sequence of SEQ ID NO: 20, or a polynucleotide having at least 95%, 96%, 97%, 98% or 99% sequence identity to SEQ ID NO: 20. In some embodiments, the modified IRES comprises a polynucleotide sequence of SEQ ID NO: 21, or a polynucleotide having at least 95%, 96%, 97%, 98% or 99% sequence identity to SEQ ID NO: 21. In some embodiments, the modified IRES comprises a polynucleotide sequence of SEQ ID NO: 22, or a polynucleotide having at least 95%, 96%, 97%, 98% or 99% sequence identity to SEQ ID NO: 22. In some embodiments, the modified IRES comprises a polynucleotide sequence of SEQ ID NO: 23, or a polynucleotide having at least 95%, 96%, 97%, 98% or 99% sequence identity to SEQ ID NO: 23. In some embodiments, the modified IRES comprises a polynucleotide sequence of SEQ ID NO: 24, or a polynucleotide having at least 95%, 96%, 97%, 98% or 99% sequence identity to SEQ ID NO: 24. In some embodiments, the modified IRES comprises a polynucleotide sequence of SEQ ID NO: 25, or a polynucleotide having at least 95%, 96%, 97%, 98% or 99% sequence identity to SEQ ID NO: 25. In some embodiments, the modified IRES comprises a polynucleotide sequence of SEQ ID NO: 33, or a polynucleotide having at least 95%, 96%, 97%, 98% or 99% sequence identity to SEQ ID NO: 33. In some embodiments, the modified IRES comprises a polynucleotide sequence of SEQ ID NO: 34, or a polynucleotide having at least 95%, 96%, 97%, 98% or 99% sequence identity to SEQ ID NO: 34. In some embodiments, the modified IRES comprises a polynucleotide sequence of SEQ ID NO: 35. In some embodiments, the modified IRES comprises a polynucleotide sequence of SEQ ID NO: 36, or a polynucleotide having at least 95%, 96%, 97%, 98% or 99% sequence identity to SEQ ID NO: 36.

In some embodiments, the modified IRES is no longer than 557 nucleotide bases in length.

In various embodiments, modified IRES, expression cassettes, vectors and host cells, described herein, are provided in provided in isolated form. This means that such modified IRES, expression cassettes, vectors and/or host cells is at least 50% w/w pure of interfering proteins, cellular and other contaminants arising from its production or purification but does not exclude the possibility that the agent is combined with an excess of pharmaceutical acceptable carrier(s) or other vehicle intended to facilitate its use. The term “isolated,” when applied to a modified IRES, expression cassette, vector and/or host cell, described herein, denotes that the modified IRES, expression cassette, vector and/or host cell is essentially free of cellular components with which it is associated in the natural state. It can be, for example, in a homogeneous state and may be in either a dry or aqueous solution. Purity and homogeneity can be determined using known methods, e.g., analytical chemistry techniques such as polyacrylamide gel electrophoresis, column chromatography, thin layer chromatography, or high-performance liquid chromatography (HPLC), mass spectrometry, flow cytometry analysis, as appropriate. An “isolated” or “purified” modified IRES, expression cassette, vector is substantially free of other cellular material, or culture medium when produced by recombinant techniques, or chemical precursors or other chemicals when chemically synthesized. In various embodiments, purified modified IRES, expression cassettes, vectors and/or host cells are at least 60%, 70%, 75%, 80%, 85%, 90%, 91%, 92%, 93%, 94%, 95%, 96%, 97%, 98%, 99%, or 100% (w/w), separated from, purified of, or free of interfering proteins and contaminants from production or purification.

Illustrative variant IRES polynucleotide sequences are provided in Table A.

TABLE A
DNA coding sequences of illustrative variant IRES277 elements

| SEQ | | |
|---|---|---|
| ID | | |
| NO: | IRES name | sequence |
| 32 | Bases 1- | ttgaaagccgggggtgggagatccggattgccagtctgctcgatatcgcaggctgggtccgtgactacccactccccctttcaa |
| | 850 in | cgtgaaggctacgatagtgccagggcgggtactgccgtaagtgccaccccaaaataacaacagacccccccccccccccccccc |
| | M81861.1 | cccccccccccccccccccccccccccccccccccccccccccccccccccccccccccccccccccccccccccccccccccc |
| | | ccccccccccctctccctcccccccccctaacgttactggccgaagccgcttggaataaggccggtgtgcgtttgtctatatgt |
| | | tattttccaccatattgccgtcttttggcaatgtgagggcccggaaacctggccctgtcttcttgacgagcattcctaggggtc |
| | | tttcccctctcgccaaaggaatgcaaggtctgttgaatgtcgtgaaggaagcagttcctctggaagcttcttgaagacaaacaa |
| | | cgtctgtagcgaccctttgcaggcagcggaaccccccacctggcgacaggtgcctctgcggccaaaagccacgtgtataagata |
| | | cacctgcaaaggcggcacaaccccagtgccacgttgtgagttggatagttgtggaaagagtcaaatggctctcctcaagcgtat |
| | | tcaacaaggggctgaaggatgcccagaaggtaccccattgtatgggatctgatctggggcctcggtgcacatgctttacatgtg |
| | | tttagtcgaggttaaaaaacgtctaggccccccgaaccacggggacgtggttttcctttgaaaaacacgatgataatatggcca |
| | | caaccatgga |
| 1 | ires277 | cccctaacgttactggccgaagccgcttggaataaggccggtgtgcgtttgtctatatgttattttccaccatattgccgtctt |
| | wt, | ttggcaatgtgagggcccggaaacctggccctgtcttcttgacgagcattcctaggggtctttcccctctcgccaaaggaatgc |
| | truncated | aaggtctgttgaatgtcgtgaaggaagcagttcctctggaagcttcttgaagacaaacaacgtctgtagcgaccctttgcaggc |
| | | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 2 | ires277- | cccctaacgttactggccgaagccgcttggaataaggccggtgtgcgtttgtctatatgttattttccaccatattgccgtctt |
| | 2G2C | ttggcaatgtgagggcccggaaacctggccctgtcttcttgacgagcattcctaccggtctttcccctctcgccaaaggaatgc |
| | | aaggtctgttgaatgtcgtgaaggaagcagttcctctggaagcttcttgaagacaaacaacgtctgtagcgaccctttgcaggc |
| | | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 3 | ires277- | cccctaacgttactggccgaagccgcttggaataaggccggtgtgcgtttgtctatatgttattttccaccatattgccgtctt |
| | 3G3C | ttggcaatgtgagggcccggaaacctggccctgtcttcttgacgagcattcctacccgtctttcccctctcgccaaaggaatgc |
| | | aaggtctgttgaatgtcgtgaaggaagcagttcctctggaagcttcttgaagacaaacaacgtctgtagcgaccctttgcaggc |
| | | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 4 | ires277- | cccctaacgttactggccgaagccgcttggaataaggccggtgtgcgtttgtctatatgttattttccaccatattgccgtctt |
| | 4G4C | ttggcaatgtgagggcccggaaacctggccctgtcttcttgacgagcattcctacccctctttcccctctcgccaaaggaatgc |
| | | aaggtctgttgaatgtcgtgaaggaagcagttcctctggaagcttcttgaagacaaacaacgtctgtagcgaccctttgcaggc |
| | | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 5 | ires277- | cccctaacgttactggccgaagccgcttggaataaggccggtgtgcgtttgtctatatgttattttccaccatattgccgtctt |
| | CAGA | ttggcaatgtgagggcccggaaacctggccctgtcttcttgacgagcattcctaggggtctttcccctctcgccaaaggaatgc |
| | | aaggtctgttgaatgtcgtgaaggaagcagttcctctggaagcttcttgaagacaaacaacgtctgtagcgaccctttgcaggc |
| | | agcggaaccccccacctgcagacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 6 | ires277- | cccctaacgttactggccgaagccgcttggaataaggccggtgtgcgtttgtctatatgttattttccaccatattgccgtctt |
| | AAAA | ttggcaatgtgagggcccggaaacctggccctgtcttcttgacgagcattcctaggggtctttcccctctcgccaaaggaatgc |
| | | aaggtctgttgaatgtcgtgaaggaagcagttcctctggaagcttcttgaagacaaacaacgtctgtagcgaccctttgcaggc |
| | | agcggaaccccccacctgaaaacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 7 | ires277- | cccctaacgttactggccgaagccgcttggaataaggccggtgtgcgtttgtctatatgttattttccaccatattgccgtctt |
| | TTAA | ttggcaatgtgagggcccggaaacctggccctgtcttcttgacgagcattcctaggggtctttcccctctcgccaaaggaatgc |
| | | aaggtctgttgaatgtcgtgaaggaagcagttcctctggaagcttcttgaagacaaacaacgtctgtagcgaccctttgcaggc |
| | | agcggaaccccccacctgttaacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 8 | ires277- | cccctaacgttactggccgaagccgcttggaataaggccggtgtgcgtttgtctatatgttattttccaccatattgccgtctt |
| | GAGA | ttggcaatgtgagggcccggaaacctggccctgtcttcttgacgagcattcctaggggtctttcccctctcgccaaaggaatgc |
| | | aaggtctgttgaatgtcgtgaaggaagcagttcctctggaagcttcttgaagacaaacaacgtctgtagcgaccctttgcaggc |
| | | agcggaaccccccacctggagacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 9 | ires277- | cccctaacgttactggccgaagccgcttggaataaggccggtgtgcgtttgtctatatgttattttccaccatattgccgtctt |
| | d419 | ttggcaatgtgagggcccggaaacctggccctgtcttcttgacgagcattcctagggg-ctttcccctctcgccaaaggaatgc |
| | (RF5) | aaggtctgttgaatgtcgtgaaggaagcagttcctctggaagcttcttgaagacaaacaacgtctgtagcgaccctttgcaggc |
| | | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 10 | ires277- | cccctaacgttactggccgaagccgcttggaataaggccggtgtgcgtttgtctatatgttattttccaccatattgccgtctt |
| | d420 | ttggcaatgtgagggcccggaaacctggccctgtcttcttgacgagcattcctaggggt-tttcccctctcgccaaaggaatgc |
| | (RF6) | aaggtctgttgaatgtcgtgaaggaagcagttcctctggaagcttcttgaagacaaacaacgtctgtagcgaccctttgcaggc |
| | | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 11 | ires277- | cccctaacgttactggccgaagccgcttggaataaggccggtgtgcgtttgtctatatgttattttccaccatattgccgtctt |
| | d422- | ttggcaatgtgagggcccggaaacctggccctgtcttcttgacgagcattcctaggggtct--cccctctcgccaaaggaatgc |
| | d423 | aaggtctgttgaatgtcgtgaaggaagcagttcctctggaagcttcttgaagacaaacaacgtctgtagcgaccctttgcaggc |
| | (RF8) | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 12 | ires277- | cccctaacgttactggccgaagccgcttggaataaggccggtgtgcgtttgtctatatgttattttccaccatattgccgtctt |
| | c424g- | ttggcaatgtgagggcccggaaacctggccctgtcttcttgacgagcattcctaggggtctttggcctctcgccaaaggaatgc |
| | c425g | aaggtctgttgaatgtcgtgaaggaagcagttcctctggaagcttcttgaagacaaacaacgtctgtagcgaccctttgcaggc |
| | (RF3) | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 13 | ires277- | cccctaacgttactggccgaagccgcttggaataaggccggtgtgcgtttgtctatatgttattttccaccatattgccgtctt |
| | c425g- | ttggcaatgtgagggcccggaaacctggccctgtcttcttgacgagcattcctaggggtctttcggctctcgccaaaggaatgc |
| | c426g | aaggtctgttgaatgtcgtgaaggaagcagttcctctggaagcttcttgaagacaaacaacgtctgtagcgaccctttgcaggc |
| | (RF4) | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 14 | ires277- | cccctaacgttactggccgaagccgcttggaataaggccggtgtgcgtttgtctatatgttattttccaccatattgccgtctt |
| | g546c | ttggcaatgtgagggcccggaaacctggccctgtcttcttgacgagcattcctaggggtctttcccctctcgccaaaggaatgc |
| | (RF9) | aaggtctgttgaatgtcgtgaaggaagcagttcctctggaagcttcttgaagacaaacaacgtctgtagcgaccctttgcaggc |
| | | agcggaaccccccacctcgcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 15 | ires277- | cccctaacgttactggccgaagccgcttggaataaggccggtgtgcgtttgtctatatgttattttccaccatattgccgtctt |
| | t545a- | ttggcaatgtgagggcccggaaacctggccctgtcttcttgacgagcattcctaggggtctttcccctctcgccaaaggaatgc |
| | g546c | aaggtctgttgaatgtcgtgaaggaagcagttcctctggaagcttcttgaagacaaacaacgtctgtagcgaccctttgcaggc |
| | (RF10) | agcggaaccccccaccacgcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 16 | ires277- | cccctaacgttactggccgaagccgcttggaataaggccggtgtgcgtttgtctatatgttattttccaccatattgccgtctt |
| | d547 | ttggcaatgtgagggcccggaaacctggccctgtcttcttgacgagcattcctaggggtctttcccctctcgccaaaggaatgc |
| | (RF11) | aaggtctgttgaatgtcgtgaaggaagcagttcctctggaagcttcttgaagacaaacaacgtctgtagcgaccctttgcaggc |
| | | agcggaaccccccacctg-cgacaggtgcctctgcggccaaaagccacgtgtataagatacacctgcaaaggggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 17 | ires277- | cccctaacgttactggccgaagccgcttggaataaggccggtgtgcgtttgtctatatgttattttccaccatattgccgtctt |
| | d549 | ttggcaatgtgagggcccggaaacctggccctgtcttcttgacgagcattcctaggggtctttcccctctcgccaaaggaatgc |
| | (RF12) | aaggtctgttgaatgtcgtgaaggaagcagttcctctggaagcttcttgaagacaaacaacgtctgtagcgaccctttgcaggc |
| | | agcggaaccccccacctggc-acaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 18 | ires277- | cccctaacgttactggccgaagccgcttggaataaggccggtgtgcgtttgtctatatgttattttccaccatattgccgtctt |
| | d550 | ttggcaatgtgagggcccggaaacctggccctgtcttcttgacgagcattcctaggggtctttcccctctcgccaaaggaatgc |
| | (RF13) | aaggtctgttgaatgtcgtgaaggaagcagttcctctggaagcttcttgaagacaaacaacgtctgtagcgaccctttgcaggc |
| | | agcggaaccccccacctggcg-caggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 19 | ires277- | cccctaacgttactggccgaagccgcttggaataaggccggtgtgcgtttgtctatatgttattttccaccatattgccgtctt |
| | c412g | ttggcaatgtgagggcccggaaacctggccctgtcttcttgacgagcattcgtaggggtctttcccctctcgccaaaggaatgc |
| | (RF1) | aaggtctgttgaatgtcgtgaaggaagcagttcctctggaagcttcttgaagacaaacaacgtctgtagcgaccctttgcaggc |
| | | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 20 | ires277- | cccctaacgttactggccgaagccgcttggaataaggccggtgtgcgtttgtctatatgttattttccaccatattgccgtctt |
| | g415c | ttggcaatgtgagggcccggaaacctggccctgtcttcttgacgagcattcctacgggtctttcccctctcgccaaaggaatgc |
| | (RF2) | aaggtctgttgaatgtcgtgaaggaagcagttcctctggaagcttcttgaagacaaacaacgtctgtagcgaccctttgcaggc |
| | | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 21 | ires277- | cccctaacgttactggccgaagccgcttggaataaggccggtgtgcgtttgtctatatgttattttccaccatattgccgtctt |
| | d421 | ttggcaatgtgagggcccggaaacctggccctgtcttcttgacgagcattcctaggggtc-ttcccctctcgccaaaggaatgc |
| | (RF7) | aaggtctgttgaatgtcgtgaaggaagcagttcctctggaagcttcttgaagacaaacaacgtctgtagcgaccctttgcaggc |
| | | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 22 | ires277 | cccctaacgttactggccgaagccgcttggaataaggccggtgtgcgtttgtctatatgttattttccaccatattgccgtctt |
| | c424g- | ttggcaatgtgagggcccggaaacctggccctgtcttcttgacgagcattcctaggggtctttggcctctcgccaaaggaatgc |
| | c425g + | aaggtctgttgaatgtcgtgaaggaagcagttcctctggaagcttcttgaagacaaacaacgtctgtagcgaccctttgcaggc |
| | G546c | agcggaaccccccacctcgcgacaggtgcctctgcggccaaaagccacgtgtataagatacacctgcaaaggcggcacaacccc |
| | (RF3n9) | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 23 | ires277 | cccctaacgttactggccgaagccgcttggaataaggccggtgtgcgtttgtctatatgttattttccaccatattgccgtctt |
| | c424g- | ttggcaatgtgagggcccggaaacctggccctgtcttcttgacgagcattcctaggggtctttggcctctcgccaaaggaatgc |
| | c425g + | aaggtctgttgaatgtcgtgaaggaagcagttcctctggaagcttcttgaagacaaacaacgtctgtagcgaccctttgcaggc |
| | D547 | agcggaaccccccacctg-cgacaggtgcctctgcggccaaaagccacgtgtataagatacacctgcaaaggcggcacaacccc |
| | (RF3n11) | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 24 | ires277 | cccctaacgttactggccgaagccgcttggaataaggccggtgtgcgtttgtctatatgttattttccaccatattgccgtctt |
| | d422- | ttggcaatgtgagggcccggaaacctggccctgtcttcttgacgagcattcctaggggtct--cccctctcgccaaaggaatgc |
| | d423 | aaggtctgttgaatgtcgtgaaggaagcagttcctctggaagcttcttgaagacaaacaacgtctgtagcgaccctttgcaggc |
| | + G546c | agcggaaccccccacctcgcgacaggtgcctctgcggccaaaagccacgtgtataagatacacctgcaaaggcggcacaacccc |
| | (RF8n9) | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 25 | ires277 | cccctaacgttactggccgaagccgcttggaataaggccggtgtgcgtttgtctatatgttattttccaccatattgccgtctt |
| | d422- | ttggcaatgtgagggcccggaaacctggccctgtcttcttgacgagcattcctaggggtct--cccctctcgccaaaggaatgc |
| | d423 | aaggtctgttgaatgtcgtgaaggaagcagttcctctggaagcttcttgaagacaaacaacgtctgtagcgaccctttgcaggc |
| | + D547 | agcggaaccccccacctg-cgacaggtgcctctgcggccaaaagccacgtgtataagatacacctgcaaaggcggcacaacccc |
| | (RF8n11) | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 33 | ires277 | cccctaacgttactggccgaagccgcttggaataaggccggtgtgcgtttgtctatatgttattttccaccatattgccgtctt |
| | 9415c + | ttggcaatgtgagggcccggaaacctggccctgtcttcttgacgagcattcctacgggtctttcccctctcgccaaaggaatgc |
| | g546c | aaggtctgttgaatgtcgtgaaggaagcagttcctctggaagcttcttgaagacaaacaacgtctgtagcgaccctttgcaggc |
| | (RF2n9) | agcggaaccccccacctcgcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 34 | ires277 | cccctaacgttactggccgaagccgcttggaataaggccggtgtgcgtttgtctatatgttattttccaccatattgccgtctt |
| | d419 + | ttggcaatgtgagggcccggaaacctggccctgtcttcttgacgagcattcctagggg-ctttcccctctcgccaaaggaatgc |
| | | aaggtctgttgaatgtcgtgaaggaagcagttcctctggaagcttcttgaagacaaacaacgtctgtagcgaccctttgcaggc |
| | g546c | agcggaaccccccacctcgcgacaggtgcctctgcggccaaaagccacgtgtataagatacacctgcaaaggcggcacaacccc |
| | (RF5n9) | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 35 | ires277 | cccctaacgttactggccgaagccgcttggaataaggccggtgtgcgtttgtctatatgttattttccaccatattgccgtctt |
| | d420 + | ttggcaatgtgagggcccggaaacctggccctgtcttcttgacgagcattcctaggggt-tttcccctctcgccaaaggaatgc |
| | g546c | aaggtctgttgaatgtcgtgaaggaagcagttcctctggaagcttcttgaagacaaacaacgtctgtagcgaccctttgcaggc |
| | (RF6n9) | agcggaaccccccacctcgcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 36 | ires277 | cccctaacgttactggccgaagccgcttggaataaggccggtgtgcgtttgtctatatgttattttccaccatattgccgtctt |
| | d421 + | ttggcaatgtgagggcccggaaacctggccctgtcttcttgacgagcattcctaggggtctt-cccctctcgccaaaggaatgc |
| | g546c | aaggtctgttgaatgtcgtgaaggaagcagttcctctggaagcttcttgaagacaaacaacgtctgtagcgaccctttgcaggc |
| | (RF7n9) | agcggaaccccccacctcgcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 37 | ires277 | cccctaacgttactggccgaagccgcttggaataaggccggtgtgcgtttgtctatatgttattttccaccatattgccgtctt |
| | d419- | ttggcaatgtgagggcccggaaacctggccctgtcttcttgacgagcattcctagggg--tttcccctctcgccaaaggaatgc |
| | d420 | aaggtctgttgaatgtcgtgaaggaagcagttcctctggaagcttcttgaagacaaacaacgtctgtagcgaccctttgcaggc |
| | (RF14) | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 38 | ires277 | cccctaacgttactggccgaagccgcttggaataaggccggtgtgcgtttgtctatatgttattttccaccatattgccgtctt |
| | c424a- | ttggcaatgtgagggcccggaaacctggccctgtcttcttgacgagcattcctaggggtctttagcctctcgccaaaggaatgc |
| | c425g | aaggtctgttgaatgtcgtgaaggaagcagttcctctggaagcttcttgaagacaaacaacgtctgtagcgaccctttgcaggc |
| | (RF15) | agcggaaccccccacctggcgacaggtgcctctgcggccaaaagccacgtgtataagatacacctgcaaaggcggcacaacccc |
| | | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 39 | ires277 | cccctaacgttactggccgaagccgcttggaataaggccggtgtgcgtttgtctatatgttattttccaccatattgccgtctt |
| | c424g- | ttggcaatgtgagggcccggaaacctggccctgtcttcttgacgagcattcctaggggtctttggcccctcgccaaaggaatgc |
| | c425g- | aaggtctgttgaatgtcgtgaaggaagcagttcctctggaagcttcttgaagacaaacaacgtctgtagcgaccctttgcaggc |
| | t428c | agcggaaccccccacctggcgacaggtgcctctgcggccaaaagccacgtgtataagatacacctgcaaaggcggcacaacccc |
| | (RF17) | agtgccacgttgtgagttggatagttgtggaaagagtcaaatggctctcctcaagcgtattcaacaaggggctgaaggatgccc |
| | | agaaggtaccccattgtatgggatctgatctggggcctcggtgcacatgctttacatgtgtttagtcgaggttaaaaaacgtct |
| | | aggccccccgaaccacggggacgtggttttcctttgaaaaacacgatgataat |
| 40 | ires277 | cccctaacgttactggccgaagccgcttggaataaggccggtgtgcgtttgtctatatgttattttccaccatattgccgtctt |
| | 546inA | ttggcaatgtgagggcccggaaacctggccctgtcttcttgacgagcattcctaggggtctttcccctctcgccaaaggaatgc |
| | (RF20) | aaggtctgttgaatgtcgtgaaggaagcagttcctctggaagcttcttgaagacaaacaacgtctgtagcgaccctttgcaggc |
| | | agcggaaccccccacctgAgcgacaggtgcctctgcggccaaaagccacgtgtataagatacacctgcaaaggcggcacaaccc |
| | | cagtgccacgttgtgagttggatagttgtggaaagagtcaaatggctctcctcaagcgtattcaacaaggggctgaaggatgcc |
| | | cagaaggtaccccattgtatgggatctgatctggggcctcggtgcacatgctttacatgtgtttagtcgaggttaaaaaacgtc |
| | | taggccccccgaaccacggggacgtggttttcctttgaaaaacacgatgataat |
| 41 | ires277 | cccctaacgttactggccgaagccgcttggaataaggccggtgtgcgtttgtctatatgttattttccaccatattgccgtctt |
| | 547inA | ttggcaatgtgagggcccggaaacctggccctgtcttcttgacgagcattcctaggggtctttcccctctcgccaaaggaatgc |
| | (RF21) | aaggtctgttgaatgtcgtgaaggaagcagttcctctggaagcttcttgaagacaaacaacgtctgtagcgaccctttgcaggc |
| | | agcggaaccccccacctggAcgacaggtgcctctgcggccaaaagccacgtgtataagatacacctgcaaaggcggcacaaccc |
| | | cagtgccacgttgtgagttggatagttgtggaaagagtcaaatggctctcctcaagcgtattcaacaaggggctgaaggatgcc |
| | | cagaaggtaccccattgtatgggatctgatctggggcctcggtgcacatgctttacatgtgtttagtcgaggttaaaaaacgtc |
| | | taggccccccgaaccacggggacgtggttttcctttgaaaaacacgatgataat |
| 42 | ires277 | cccctaacgttactggccgaagccgcttggaataaggccggtgtgcgtttgtctatatgttattttccaccatattgccgtctt |
| | 549inA | ttggcaatgtgagggcccggaaacctggccctgtcttcttgacgagcattcctaggggtctttcccctctcgccaaaggaatgc |
| | (RF23) | aaggtctgttgaatgtcgtgaaggaagcagttcctctggaagcttcttgaagacaaacaacgtctgtagcgaccctttgcaggc |
| | | agcggaaccccccacctggcgAacaggtgcctctgcggccaaaagccacgtgtataagatacacctgcaaaggcggcacaaccc |
| | | cagtgccacgttgtgagttggatagttgtggaaagagtcaaatggctctcctcaagcgtattcaacaaggggctgaaggatgcc |
| | | cagaaggtaccccattgtatgggatctgatctggggcctcggtgcacatgctttacatgtgtttagtcgaggttaaaaaacgtc |
| | | taggccccccgaaccacggggacgtggttttcctttgaaaaacacgatgataat |

### 3. Expression Cassettes

Further provided are expression cassettes comprising the modified IRES sequences, described herein. Generally, the expression cassettes comprise one or more iterations of the following operably linked elements in the 5′ to 3′ direction: promoter-coding sequence of protein of interest (CDS)-IRES-selection marker-polyA signal sequence. The term “operably linked” refers to two or more (i.e., at least two) nucleic acid sequence or polypeptide elements that are usually physically linked and are in a functional relationship with each other. Operably linked elements can but need not directly abut one another. For instance, a promoter is operably linked to a coding sequence if the promoter is able to initiate or regulate the transcription or expression of a coding sequence, in which case, the coding sequence should be understood as being “under the control of” the promoter.

With respect to promoters, in various embodiments, the promoter for expression of the proteins of interest (e.g., antibodies, Fc fusion proteins, multi-specific binding molecules) in mammalian cells is a constitutive promoter or an inducible promoter. Illustrative promoters for expression of the proteins of interest in mammalian cells include without limitation an SV40 promoter (Mulligan et al., Nature, 277:108 (1979)), MMLV-LTR promoter, an elongation-factor 1α subunit (EF-1α) promoter (Mizushima et al., Nucleic Acids Res., 18:5322 (1990)), a cytomegalovirus (CMV) promoter; a cytomegalovirus (CMV) early enhancer element (C)-first exon and the first intron of chicken beta-actin gene (A)-splice acceptor of the rabbit beta-globin gene (G) (CAG) promoter (Miyazaki, et al., Gene (1989) 79(2):269-77; Niwa, et al., Gene (1991) 108(2):193-9); a phosphoglycerokinase (PGK) promoter, a human neutrophil specific α-defensin (DEF) promoter, a ubiquitin C (UbC) promoter, and a beta-actin promoter. See, e.g., Qin, et al., (2010) PLOS ONE 5(5): e10611.

With respect to selection markers, in various embodiments, the selection marker is a polynucleotide encoding a glutamine synthetase (GS) or an antibiotic resistance protein. In some embodiments, the selection marker is an antibiotic resistance protein encoded by NeoR, BsdR, HygR, PuroR, BleoR (a.k.a., ZeoR). See, Tsai, et al., J Biol Chem. (2022) 298(5):101846. In some embodiments, the selection marker is a polynucleotide encoding a glutamine synthetase (GS) enzyme. In some embodiments, GS is selected from a human GS, a fish GS, a koala GS, a hamster GS and variants thereof. In various embodiments, the GS is encoded by a polynucleotide of SEQ ID NOs: 26-29 or a polynucleotide having at least 90%, 91%, 92%, 93%, 94%, 95%, 96%, 97%, 98%, 99% sequence identity to a polynucleotide of SEQ ID NOs: 26-29. In various embodiments, the GS is a koala GS encoded by a polynucleotide of SEQ ID NO: 26 or a polynucleotide having at least 90%, 91%, 92%, 93%, 94%, 95%, 96%, 97%, 98%, 99% sequence identity to a polynucleotide of SEQ ID NOs: 26. In various embodiments, the GS is a fish GS encoded by a polynucleotide of SEQ ID NO: 27 or a polynucleotide having at least 90%, 91%, 92%, 93%, 94%, 95%, 96%, 97%, 98%, 99% sequence identity to a polynucleotide of SEQ ID NOs: 27. In various embodiments, the GS is a hamster GS encoded by a polynucleotide of SEQ ID NO: 28 or a polynucleotide having at least 90%, 91%, 92%, 93%, 94%, 95%, 96%, 97%, 98%, 99% sequence identity to a polynucleotide of SEQ ID NOs: 28. In various embodiments, the GS is a human GS encoded by a polynucleotide of SEQ ID NO: 29 or a polynucleotide having at least 90%, 91%, 92%, 93%, 94%, 95%, 96%, 97%, 98%, 99% sequence identity to a polynucleotide of SEQ ID NOs: 29. In some embodiments, the GS is a variant human GS. In some embodiments the human variant GS is R324S (SEQ ID NO: 30). Illustrative GS-encoding polynucleotide sequences of use in the herein described expression cassettes are provided in Table B, below.

With respect to polyadenylation (polyA) signal sequences, a polyA signal sequence of use generally have two recognition elements: (i) an essentially invariant AAUAAA hexamer, and (ii) a more variable element rich in U or GU residues located approximately 20-50 nucleotides downstream of (3′ to) the essentially invariant AAUAAA hexamer (SEQ ID NO: 31). Illustrative polyA signal sequences of use include SV40 polyA signal sequence and bovine growth hormone (BGH) polyA signal sequence.

In some embodiments, the expression cassette comprises from 5′ to 3′ the following operably linked elements:
1) A promoter;
2) A polynucleotide encoding a protein of interest;
3) A modified IRES sequence, as described herein, e.g., of any one of SEQ ID NOs: 3-4, 9-25 and 33-42, e.g., of any one of SEQ ID NOs: 12-14, 17, 22, 24, 40 and 42; 4) A selection marker; and
5) A poly adenosine (polyA) signal sequence. In various embodiments, the protein of interest can be an immunoglobulin heavy chain, an immunoglobulin light chain or an Fc-fusion protein.

TABLE B
Illustrative glutamine synthetase (GS) coding sequences

| SEQ | | |
|---|---|---|
| ID | GS | |
| NO: | species | sequence |
| 26 | koala | atggctacctctgcctccagccacctgaacaagaacatcaagcagatgtacatgaacctgcctcagggcaacaaggtgctggcc |
| | | atgtatatctggatcgacggcaccggcgagggcctgagatgcaagacaagaaccctggactccgagcctaagtccatcgacgat |
| | | ctgcccgagtggaacttcgacggctcctccacctatcagtccgagggctccaactccgacatgtacttggtgcctgccgctctg |
| | | ttcagggaccctttcagaaaggaccccaacaagctggtgttctgcgaggtgttcaagtacaaccggaagcctgccgagacaaac |
| | | ctgcggcatacctgcaagcggatcatggacatggtcaccaaccagaaaccttggtttggcatggaacaagagtacaccctgatg |
| | | ggcaccgacggccatccttttggctggccttctaacggcttccctggacctcagggcccttactattgtggcgtgggcgccgat |
| | | aaggcctacggcagagatatcgctgaggcccactacagagcctgtctgtacgctggcgtgaagatcgtgggcaccaatgccgag |
| | | gctatgccctctcagtgggagttccagatcggcccttgtgaaggcatcgagatgggcgatcatctgtgggtcgcccggttcatc |
| | | ctgcacagagtgtgcgaggacttcggcatgatcgtgaccttcgatcccaagcctattcctggcaactggaacggcgctggctgc |
| | | cacaccaacttctctaccaaggccatgcgggaagagaacggcctgaagttcatcgaggaagccatcgagcggctgtccaagcgg |
| | | cacaagtaccacatcagagcctacgatcccaaaggcggcctggacaatgctagaaggctgaccggcttcaacgagacatccaac |
| | | atcaacgagttctctgccggcgtggccaacagaggcgccagcatcagaatccccagaatggtcggacaggacaagaagggctac |
| | | ttcgaggacagacggccttccgccaactgcgatccttacgctgtgacagaggccctgatccggacctgcctgttgaatgagaca |
| | | ggcgaggaacccttccagtacaagaac |
| 27 | fish | atggctacctccgcctcctccaagctgtccaaggctatgaagcagcagtacatggacctgcctcagggcgagaaggtgcaagtg |
| | | atgtatatctggatcgacggcaccggcgagggcctgagatgcaagacaagaaccctggactccgagcctaagtccgtggaagaa |
| | | ctgcccgagtggaacttcgacggctccagcacatatcaggccgagggctccaactccgacatgtatctgttccctcaagccatg |
| | | ttcagggaccctttccggaaggaccccaacaagctggtgctgtgcgacgtgctgaagtacaaccacaagcctgccgagacaaac |
| | | ctgcggcagtcctgcaagaaaatcatggacatggtgcagaatcagcacccttggtttggcatggaacaagagtacaccctgctg |
| | | ggcaccgacggccatccttttggctggccttctaacggcttccctggacctcagggcccttactattgtggcgtgggcgccgat |
| | | aaggcctacggcagagatgtggtggaagcccactacagagcctgtctgtacgctggcgtgaagatctgtggcaccaacgccgaa |
| | | gtgatgcccgctcagtgggagtttcaagtgggcccttgtgaaggcatcgagatgggcgatcatctgtgggtcgccagattcctg |
| | | ctgcacagagtgtgcgaggatttcggcgtggtggccagcttcgaccccaagcctattcctggcaactggaacggcgctggctgc |
| | | cacaccaacttctccaccaaagagatgcgcgaggaaggcggcctgaagcacatcgaggaatccatcgagaagctgggcaagaga |
| | | caccagtaccacatccgggcctacgatcccaaaggcggactggataatgccagaaggctgaccggccaccatgagacatccaac |
| | | atcaacgagttctctgccggcgtggccaacagaggcgccagcatcagaatccctagatccgtgggccaagagaagaagggctac |
| | | ttcgaggacagacggccttccgccaactgcgatccttacgctgtgaccgaggctctgatccggacctgtctgctgaacgaagag |
| | | ggcgaagtgcccgagaagtacatg |
| 28 | hamster | atggccacctcagcaagttcccacttgaacaaaaacatcaagcaaatgtacttgtgcctgccccagggtgagaaagtccaagcc |
| | | atgtatatctgggttgatggtactggagaaggactgcgctgcaaaacccgcaccctggactgtgagcccaagtgtgtagaagag |
| | | ttacctgagtggaattttgatggctctagtacctttcagtctgagggctccaacagtgacatgtatctcagccctgttgccatg |
| | | tttcgggaccccttccgcagagatcccaacaagctggtgttctgtgaagttttcaagtacaaccggaagcctgcagagaccaat |
| | | ttaaggcactcgtgtaaacggataatggacatggtgagcaaccagcacccctggtttggaatggaacaggagtatactctgatg |
| | | ggaacagatgggcacccttttggttggccttccaatggctttcctgggccccaaggtccgtattactgtggtgtgggcgcagac |
| | | aaagcctatggcagggatatcgtggaggctcactaccgcgcctgcttgtatgctggggtcaagattacaggaacaaatgctgag |
| | | gtcatgcctgcccagtgggagttccaaataggaccctgtgaaggaatccgcatgggagatcatctctgggtggcccgtttcatc |
| | | ttgcatcgagtatgtgaagactttggggtaatagcaacctttgaccccaagcccattcctgggaactggaatggtgcaggctgc |
| | | cataccaactttagcaccaaggccatgcgggaggagaatggtctgaagcacatcgaggaggccatcgagaaactaagcaagcgg |
| | | caccggtaccacattcgagcctacgatcccaaggggggcctggacaatgcccgtcgtctgactgggttccacgaaacgtccaac |
| | | atcaacgacttttctgctggtgtcgccaatcgcagtgccagcatccgcattccccggactgtcggccaggagaagaaaggttac |
| | | tttgaagaccgccgcccctctgccaattgtgacccctttgcagtgacagaagccatcgtccgcacatgccttctcaatgagact |
| | | ggcgacgagcccttccaatacaaaaac |
| 29 | human | atgaccacctcagcaagttcccacttaaataaaggcatcaagcaggtgtacatgtccctgcctcagggtgagaaagtccaggcc |
| | | atgtatatctggatcgatggtactggagaaggactgcgctgcaagacccggaccctggacagtgagcccaagtgtgtggaagag |
| | | ttgcctgagtggaatttcgatggctctagtactttacagtctgagggttccaacagtgacatgtatctcgtgcctgctgccatg |
| | | tttcgggaccccttccgtaaggaccctaacaagctggtgttatgtgaagttttcaagtacaatcgaaggcctgcagagaccaat |
| | | ttgaggcacacctgtaaacggataatggacatggtgagcaaccagcacccctggtttggcatggagcaggagtataccctcatg |
| | | gggacagatgggcacccctttggttggccttccaacggcttcccagggccccagggtccatattactgtggtgtgggagcagac |
| | | agagcctatggcagggacatcgtggaggcccattaccgggcctgcttgtatgctggagtcaaaattgcggggactaatgccgag |
| | | gtcatgcctgcccagtgggaatttcagattggaccttgtgaaggaatcagcatgggagatcatctctgggtggcccgtttcatc |
| | | ttgcatcgtgtgtgtgaagactttggagtgatagcaacctttgatcctaagcccattcctgggaactggaatggtgcaggctgc |
| | | cataccaacttcagcaccaaggccatgcgggaggagaatggtctgaagtacatcgaggaggccattgagaaactaagcaagcgg |
| | | caccagtaccacatccgtgcctatgatcccaagggaggcctggacaatgcccgacgtctaactggattccatgaaacctccaac |
| | | atcaacgacttttctgctggtgtagccaatcgtagcgccagcatacgcattccccggactgttggccaggagaagaagggttac |
| | | tttgaagatcgtcgcccctctgccaactgcgaccccttttcggtgacagaagccctcatccgcacgtgtcttctcaatgaaacc |
| | | ggcgatgagcccttccagtacaaaaat |
| 30 | human- | atgaccacctcagcaagttcccacttaaataaaggcatcaagcaggtgtacatgtccctgcctcagggtgagaaagtccaggcc |
| | GS- | atgtatatctggatcgatggtactggagaaggactgcgctgcaagacccggaccctggacagtgagcccaagtgtgtggaagag |
| | R324S | ttgcctgagtggaatttcgatggctctagtactttacagtctgagggttccaacagtgacatgtatctcgtgcctgctgccatg |
| | | tttcgggaccccttccgtaaggaccctaacaagctggtgttatgtgaagttttcaagtacaatcgaaggcctgcagagaccaat |
| | | ttgaggcacacctgtaaacggataatggacatggtgagcaaccagcacccctggtttggcatggagcaggagtataccctcatg |
| | | gggacagatgggcacccctttggttggccttccaacggcttcccagggccccagggtccatattactgtggtgtgggagcagac |
| | | agagcctatggcagggacatcgtggaggcccattaccgggcctgcttgtatgctggagtcaaaattgcggggactaatgccgag |
| | | gtcatgcctgcccagtgggaatttcagattggaccttgtgaaggaatcagcatgggagatcatctctgggtggcccgtttcatc |
| | | ttgcatcgtgtgtgtgaagactttggagtgatagcaacctttgatcctaagcccattcctgggaactggaatggtgcaggctgc |
| | | cataccaacttcagcaccaaggccatgcgggaggagaatggtctgaagtacatcgaggaggccattgagaaactaagcaagcgg |
| | | caccagtaccacatccgtgcctatgatcccaagggaggcctggacaatgcccgacgtctaactggattccatgaaacctccaac |
| | | atcaacgacttttctgctggtgtagccaatcgtagcgccagcatatccattccccggactgttggccaggagaagaagggttac |
| | | tttgaagatcgtcgcccctctgccaactgcgaccccttttcggtgacagaagccctcatccgcacgtgtcttctcaatgaaacc |
| | | ggcgatgagcccttccagtacaaaaat |

a. Biologic Molecule Having One Polypeptide Chain

In some embodiments, comprises elements for expressing a protein of interest (i.e., a biologic molecule) comprising one polypeptide chain. Illustrative biologic molecules expressed from an expression cassette having a coding sequence for a single polypeptide chain include without limitation, cytokines, polypeptide hormones, Fc fusion proteins (e.g., cytokine-Fc, Fc-cytokine, scFv-Fc, and immunoadhesins).

In some embodiments, an expression cassette for expressing a protein of interest (POI) having a single polypeptide chain comprises the following operably linked elements in the 5′ to 3′ direction: promoter-POI CDS-variant IRES as described herein, e.g., of any one of SEQ ID NOs: 3-4, 9-25 and 33-42-selection marker CDS-polyA signal sequence. In some embodiments, an expression cassette for expressing a protein of interest (POI) having a single polypeptide chain comprises the following operably linked elements in the 5′ to 3′ direction: promoter-POI CDS-variant IRES as described herein, e.g., of any one of SEQ ID NOs: 3-4, 9-25 and 33-42-GS CDS-polyA signal sequence. In some embodiments, an expression cassette for expressing a protein of interest (POI) having a single polypeptide chain comprises the following operably linked elements in the 5′ to 3′ direction: CMV promoter-POI CDS-variant IRES as described herein, e.g., of any one of SEQ ID NOs: 3-4, 9-25 and 33-42, as described herein-human R324S variant GS CDS-SV40 polyA signal sequence. In some embodiments, an expression cassette for expressing a protein of interest (POI) having a single polypeptide chain comprises the following operably linked elements in the 5′ to 3′ direction: CMV promoter-POI CDS-variant IRES of any one of SEQ ID NOs: 12-14, 17, 22, 24, 40 and 42, as described herein-human R324S variant GS CDS-SV40 polyA signal sequence. In some embodiments, an expression cassette for expressing an Fc fusion protein comprises the following operably linked elements in the 5′ to 3′ direction: CMV promoter-Fc fusion protein CDS-variant IRES of any one of SEQ ID NOs: 12-14, 17, 22, 24, 40 and 42, as described herein-human R324S variant GS CDS-SV40 polyA signal sequence.

In some embodiments, provided is an expression cassette comprising from 5′ to 3′ the following operably linked elements:
1) A promoter;
2) A polynucleotide encoding a polypeptide of interest;
3) A modified IRES as described herein, e.g., of any one of SEQ ID NOs: 3-4, 9-25 and 33-42, e.g., of any one of SEQ ID NOs: 12-14, 17, 22, 24, 40 and 42;
4) A selection marker, e.g., a polynucleotide encoding a GS, e.g., human variant GS is R324S (SEQ ID NO:30) or an antibiotic resistance gene, e.g., encoded by BleoR; and
) A polyA signal sequence. In various embodiments, the protein of interest can be an immunoglobulin heavy chain, an immunoglobulin light chain or an Fc-fusion protein.
   b. Biologic Molecule Having Two Polypeptide Chains

Further provided are sets of two or more expression cassettes comprising elements for expressing a protein of interest (i.e., a biologic molecule) comprising two polypeptide chains. A set of two or more expression cassettes can be in one (e.g., a multi-cistronic, e.g., a bicistronic vector) or two vectors. Illustrative biologic molecules expressed from a set of two or more expression cassettes having coding sequences for two polypeptide chains include without limitation, monospecific antibodies, monoclonal antibodies, scFv-Fc x scFv-Fc bispecific molecules, Fc fusion protein x scFv-Fc heterodimers, Fc fusion protein x Fc fusion protein heterodimers.

In some embodiments, the set of expression cassettes comprises from 5′ to 3′ the following operably linked elements:
1) A first promoter;
2) A first polynucleotide encoding a first polypeptide of the protein of interest; 3) A first polyA signal sequence;
4) A second promoter;
5) A second polynucleotide encoding a second polypeptide of the protein of interest; 6) A modified IRES as described herein, e.g., of any one of SEQ ID NOs: 3-4, 9-25 and 33-42, e.g., of any one of SEQ ID NOs: 12-14, 17, 22, 24, 40 and 42;
7) A selection marker, e.g., a polynucleotide encoding a GS, e.g., human variant GS is R324S (SEQ ID NO:30) or an antibiotic resistance gene, e.g., encoded by BleoR; and 8) A second polyA signal sequence.

In some embodiments, the set of expression cassettes comprises from 5′ to 3′ the following operably linked elements:
1) A first promoter;
2) A first polynucleotide encoding a first polypeptide of the protein of interest; 3) A modified IRES as described herein, e.g., of any one of SEQ ID NOs: 3-4, 9-25 and 33-42, e.g., of any one of SEQ ID NOs: 12-14, 17, 22, 24, 40 and 42;
4) A selection marker, e.g., a polynucleotide encoding a GS, e.g., human variant GS is R324S (SEQ ID NO:30) or an antibiotic resistance gene, e.g., encoded by BleoR;
) A first polyA signal sequence;
6) A second promoter;
7) A second polynucleotide encoding a second polypeptide of the protein of interest; and
8) A second polyA signal sequence.

In some embodiments, the set of expression cassettes comprises from 5′ to 3′ the following operably linked elements:
1) A first promoter;
2) A first polynucleotide encoding a first polypeptide of the protein of interest; 3) A first modified IRES as described herein, e.g., of any one of SEQ ID NOs: 3-4, 9-25 and 33-42, e.g., of any one of SEQ ID NOs: 12-14, 17, 22, 24, 40 and 42; 4) A first selection marker, e.g., a polynucleotide encoding a GS, e.g., human variant GS is R324S (SEQ ID NO:30) or an antibiotic resistance gene, e.g., encoded by BleoR; 5) A first polyA signal sequence;
6) A second promoter;
7) A second polynucleotide encoding a second polypeptide of the protein of interest; 8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
9) A second selection marker, e.g., a polynucleotide encoding an antibiotic resistance gene, e.g., a polynucleotide encoding an antibiotic resistance gene, e.g., optionally, wherein the second selection marker is different from the first selection marker; 10) A second polyA signal sequence.

In some embodiments, the set of expression cassettes comprises from 5′ to 3′ the following operably linked elements:
1) A first promoter;
2) A first polynucleotide encoding an antibody light chain;
3) A first polyA signal sequence;
4) A second promoter;
5) A second polynucleotide encoding an antibody heavy chain;
6) The modified IRES as described herein, e.g., of any one of SEQ ID NOs: 3-4, 9-25 and 33-42, e.g., of any one of SEQ ID NOs: 12-14, 17, 22, 24, 40 and 42;
7) A selection marker, e.g., a polynucleotide encoding a GS, e.g., human variant GS is R324S (SEQ ID NO:30) or an antibiotic resistance gene, e.g., encoded by BleoR; and 8) A second polyA signal sequence.

In some embodiments, the set of expression cassettes comprises from 5′ to 3′ the following operably linked elements:
1) A first promoter;
2) A first polynucleotide encoding an antibody heavy chain;
3) A first polyA signal sequence;
4) A second promoter;
5) A second polynucleotide encoding an antibody light chain;
6) A modified IRES as described herein, e.g., of any one of SEQ ID NOs: 3-4, 9-25 and 33-42, e.g., of any one of SEQ ID NOs: 12-14, 17, 22, 24, 40 and 42;
7) A selection marker, e.g., a polynucleotide encoding a GS, e.g., human variant GS is R324S (SEQ ID NO:30) or an antibiotic resistance gene, e.g., encoded by BleoR; and 8) A second polyA signal sequence.

In some embodiments, the set of expression cassettes comprises from 5′ to 3′ the following operably linked elements:
1) A first promoter;
2) A first polynucleotide encoding an antibody light chain;
3) A modified IRES as described herein, e.g., of any one of SEQ ID NOs: 3-4, 9-25 and 33-42, e.g., of any one of SEQ ID NOs: 12-14, 17, 22, 24, 40 and 42;
4) A selection marker, e.g., a polynucleotide encoding a GS, e.g., human variant GS is R324S (SEQ ID NO:30) or an antibiotic resistance gene, e.g., encoded by BleoR; 5) A first polyA signal sequence;
6) A second promoter;
7) A second polynucleotide encoding an antibody heavy chain; and
8) A second polyA signal sequence.

In some embodiments, the set of expression cassettes comprises from 5′ to 3′ the following operably linked elements:
1) A first promoter;
2) A first polynucleotide encoding an antibody heavy chain;
3) A modified IRES as described herein, e.g., of any one of SEQ ID NOs: 3-4, 9-25 and 33-42, e.g., of any one of SEQ ID NOs: 12-14, 17, 22, 24, 40 and 42;
4) A selection marker, e.g., a polynucleotide encoding a GS, e.g., human variant GS is R324S (SEQ ID NO:30) or an antibiotic resistance gene, e.g., encoded by BleoR;
) A first polyA signal sequence;
6) A second promoter;
7) A second polynucleotide encoding an antibody light chain; and
8) A second polyA signal sequence.

In some embodiments, the set of expression cassettes comprises from 5′ to 3′ the following operably linked elements:
1) A first promoter;
2) A first polynucleotide encoding an antibody light chain;
3) A first modified IRES as described herein, e.g., of any one of SEQ ID NOs: 3-4, 9-25 and 33-42, e.g., of any one of SEQ ID NOs: 12-14, 17, 22, 24, 40 and 42; 4) A first selection marker, e.g., a polynucleotide encoding a GS, e.g., human variant GS is R324S (SEQ ID NO:30) or an antibiotic resistance gene, e.g., encoded by BleoR; 5) A first polyA signal sequence;
6) A second promoter;
7) A second polynucleotide encoding an antibody heavy chain;
8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
9) A second selection marker, e.g., a polynucleotide encoding an antibiotic resistance gene, optionally, wherein the second selection marker is different from the first selection marker; and 10) A second polyA signal sequence.

In some embodiments, the set of expression cassettes comprises from 5′ to 3′ the following operably linked elements:
1) A first promoter;
2) A first polynucleotide encoding an antibody heavy chain;
3) A first modified IRES as described herein, e.g., of any one of SEQ ID NOs: 3-4, 9-25 and 33-42, e.g., of any one of SEQ ID NOs: 12-14, 17, 22, 24, 40 and 42; 4) A first selection marker, e.g., a polynucleotide encoding a GS, e.g., human variant GS is R324S (SEQ ID NO:30) or an antibiotic resistance gene, e.g., encoded by BleoR;
) A first polyA signal sequence;
6) A second promoter;
7) A second polynucleotide encoding an antibody light chain;
8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
9) A second selection marker, e.g., a polynucleotide encoding an antibiotic resistance gene, optionally, wherein the second selection marker is different from the first selection marker; and
) A second polyA signal sequence.

In various embodiments, the first promoter and the second promoter are the same. In some embodiments, the first promoter and the second promoter are different. In some embodiments, the first promoter and the second promoter induce equivalent expression levels of a 3′ protein of interest. In some embodiments, the first promoter and the second promoter induce different expression levels of a 3′ protein of interest.

In various embodiments, the first modified IRES and the second modified IRES are the same. In various embodiments, the first modified IRES and the second modified IRES are different.

In some embodiments, the first selection marker and the second selection marker are different. For example, in some embodiments, the first selection marker comprises a polynucleotide encoding a GS and the second selection marker comprises a polynucleotide encoding an antibiotic resistance gene. In some embodiments, the first selection marker comprises a polynucleotide encoding an antibiotic resistance gene and the second selection marker comprises a polynucleotide encoding a GS. In some embodiments, the selection marker is an antibiotic resistance protein, e.g., encoded by NeoR, BsdR, HygR, PuroR, BleoR (a.k.a., ZeoR). In some embodiments, the selection marker is an antibiotic resistance protein encoded by BleoR. In some embodiments, the selection marker is a polynucleotide encoding a glutamine synthetase (GS) enzyme. In some embodiments, GS is selected from a human GS, a fish GS, a koala GS, a hamster GS and variants thereof. In some embodiments, the GS is a variant human GS. In some embodiments the human variant GS is R324S (SEQ ID NO:30). In some embodiments, the first selection marker and the second selection marker are the same, e.g., when on different vectors or when on expression cassettes transfected into different host cells.

In various embodiments, the first polyA signal sequence and the second polyA signal sequence are the same. In various embodiments, the first polyA signal sequence and the second polyA signal sequence are different.

In various embodiments, the first expression cassette containing the first polynucleotide and the second expression cassette containing the second polynucleotide are in the same vector. In various embodiments, the first expression cassette containing the first polynucleotide and the second expression cassette containing the second polynucleotide are in different vectors.

c. Biologic Molecule Having Three Polypeptide Chains

Further provided are sets of three or more expression cassettes comprising elements for expressing a protein of interest (i.e., a biologic molecule) comprising three polypeptide chains. A set of three or more expression cassettes can be in one (e.g., a tricistronic vector), two (e.g., a bicistronic and a monocistronic vector) or three vectors (e.g., three monocistronic vectors). Illustrative biologic molecules expressed from a set of three or more expression cassettes having coding sequences for three polypeptide chains include without limitation, Fab-Fc x scFv-Fc heterodimers, Fc fusion protein x Fab-Fc heterodimers. Biologic molecules having three polypeptide chains can have an immunoglobulin heavy chain, an immunoglobulin light chain and an Fc fusion protein (e.g., a cytokine-Fc fusion protein, a receptor extracellular domain-Fc fusion protein, an scFv fusion protein). Illustrative tricistronic vectors for expressing multi-specific biologic molecules are described, e.g., in WO 2022/046644.

In some embodiments, the set of expression cassettes comprises from 5′ to 3′ the following operably linked elements:
1) A first promoter;
2) A first polynucleotide encoding a first polypeptide of the protein of interest;
3) A first polyA signal sequence;
4) A second promoter;
5) A second polynucleotide encoding a second polypeptide of the protein of interest;
6) A second polyA signal sequence;
7) A third promoter;
8) A third polynucleotide encoding a third polypeptide of the protein of interest;
9) A modified IRES as described herein, e.g., of any one of SEQ ID NOs: 3-4, 9-25 and 33-42, e.g., of any one of SEQ ID NOs: 12-14, 17, 22, 24, 40 and 42;
10) A selection marker, e.g., a polynucleotide encoding a GS or a polynucleotide encoding an antibiotic resistance gene; and
11) A third polyA signal sequence.

In some embodiments, the set of expression cassettes comprises from 5′ to 3′ the following operably linked elements:
1) A first promoter;
2) A first polynucleotide encoding a first polypeptide of the protein of interest;
3) A modified IRES as described herein, e.g., of any one of SEQ ID NOs: 3-4, 9-25 and 33-42, e.g., of any one of SEQ ID NOs: 12-14, 17, 22, 24, 40 and 42;
4) A selection marker, e.g., a polynucleotide encoding a GS or a polynucleotide encoding an antibiotic resistance gene;
5) A first polyA signal sequence;
6) A second promoter;
7) A second polynucleotide encoding a second polypeptide of the protein of interest;
8) A second polyA signal sequence;
9) A third promoter;
10) A third polynucleotide encoding a third polypeptide of the protein of interest; and
11) A third polyA signal sequence.

In some embodiments, the set of expression cassettes comprises from 5′ to 3′ the following operably linked elements:
1) A first promoter;
2) A first polynucleotide encoding a first polypeptide of the protein of interest;
3) A first polyA signal sequence;
4) A second promoter;
5) A second polynucleotide encoding a second polypeptide of the protein of interest;
6) A modified IRES as described herein, e.g., of any one of SEQ ID NOs: 3-4, 9-25 and 33-42, e.g., of any one of SEQ ID NOs: 12-14, 17, 22, 24, 40 and 42;
7) A selection marker, e.g., a polynucleotide encoding a GS or a polynucleotide encoding an antibiotic resistance gene;
8) A second polyA signal sequence;
9) A third promoter;
10) A third polynucleotide encoding a third polypeptide of the protein of interest; and
11) A third polyA signal sequence.

In some embodiments, the set of expression cassettes comprises from 5′ to 3′ the following operably linked elements:
1) A first promoter;
2) A first polynucleotide encoding a first polypeptide of the protein of interest;
3) A first modified IRES as described herein, e.g., of any one of SEQ ID NOs: 3-4, 9-25 and 33-42, e.g., of any one of SEQ ID NOs: 12-14, 17, 22, 24, 40 and 42;
4) A first selection marker, e.g., a polynucleotide encoding a GS or a polynucleotide encoding an antibiotic resistance gene;
5) A first polyA signal sequence;
6) A second promoter;
7) A second polynucleotide encoding a second polypeptide of the protein of interest;
8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
9) A second selection marker, optionally, wherein the second selection marker is different from the first selection marker;
10) A second polyA signal sequence;
11) A third promoter;
12) A third polynucleotide encoding a third polypeptide of the protein of interest;
13) A third modified IRES, optionally comprising a polynucleotide of any one of SEQ ID NOs: 3-4, 9-25 and 33-42, e.g., of any one of SEQ ID NOs: 12-14, 17, 22, 24, 40 and 42;
14) A third selection marker, optionally, wherein the third selection marker is different from the first selection marker and the second selection marker; and
15) A third polyA signal sequence.

In some embodiments, the set of expression cassettes comprises from 5′ to 3′ the following operably linked elements:
1) A first promoter;
2) A first polynucleotide encoding an antibody light chain;
3) A first polyA signal sequence;
4) A second promoter;
5) A second polynucleotide encoding an antibody heavy chain;
6) A modified IRES as described herein, e.g., of any one of SEQ ID NOs: 3-4, 9-25 and 33-42, e.g., of any one of SEQ ID NOs: 12-14, 17, 22, 24, 40 and 42;
7) A selection marker, e.g., a polynucleotide encoding a GS or a polynucleotide encoding an antibiotic resistance gene;
8) A second polyA signal sequence;
9) A third promoter;
10) A third polynucleotide encoding an Fc fusion protein or a scFv-Fc fusion protein; and
11) A third polyA signal sequence.

In some embodiments, the set of expression cassettes comprises from 5′ to 3′ the following operably linked elements:
1) A first promoter;
2) A first polynucleotide encoding an antibody light chain;
3) A first polyA signal sequence;
4) A second promoter;
5) A second polynucleotide encoding an antibody heavy chain;
6) A second polyA signal sequence;
7) A third promoter;
8) A third polynucleotide encoding an Fc fusion protein or a scFv-Fc fusion protein;
9) A modified IRES as described herein, e.g., of any one of SEQ ID NOs: 3-4, 9-25 and 33-42, e.g., of any one of SEQ ID NOs: 12-14, 17, 22, 24, 40 and 42;
10) A selection marker, e.g., a polynucleotide encoding a GS or a polynucleotide encoding an antibiotic resistance gene; and
11) A third polyA signal sequence.

In some embodiments, the set of expression cassettes comprises from 5′ to 3′ the following operably linked elements:
1) A first promoter;
2) A first polynucleotide encoding an antibody light chain;
3) A first polyA signal sequence;
4) A second promoter;
5) A second polynucleotide encoding an antibody heavy chain;
6) A first modified IRES as described herein, e.g., of any one of SEQ ID NOs: 3-4, 9-25 and 33-42, e.g., of any one of SEQ ID NOs: 12-14, 17, 22, 24, 40 and 42;
7) A first selection marker, e.g., a polynucleotide encoding a GS or a polynucleotide encoding an antibiotic resistance gene;
8) A second polyA signal sequence;
9) A third promoter;
10) A third polynucleotide encoding an Fc fusion protein or a scFv-Fc fusion protein;
11) A second modified IRES, optionally comprising a polynucleotide of any one of SEQ ID NOs: 3-4, 9-25 and 33-42, e.g., of any one of SEQ ID NOs: 12-14, 17, 22,24,40 and 42;
12) A second selection marker, optionally, wherein the second selection marker is different from the first selection marker; and
13) A third polyA signal sequence.

In some embodiments, the set of expression cassettes comprises from 5′ to 3′ the following operably linked elements:
1) A first promoter;
2) A first polynucleotide encoding an antibody heavy chain;
3) A first polyA signal sequence;
4) A second promoter;
5) A second polynucleotide encoding an antibody light chain;
6) A modified IRES as described herein, e.g., of any one of SEQ ID NOs: 3-4, 9-25 and 33-42, e.g., of any one of SEQ ID NOs: 12-14, 17, 22, 24, 40 and 42;
7) A selection marker, e.g., a polynucleotide encoding a GS or a polynucleotide encoding an antibiotic resistance gene;
8) A second polyA signal sequence;
9) A third promoter;
10) A third polynucleotide encoding an Fc fusion protein or a scFv-Fc fusion protein; and
11) A third polyA signal sequence.

In some embodiments, the set of expression cassettes comprises from 5′ to 3′ the following operably linked elements:
1) A first promoter;
2) A first polynucleotide encoding an antibody heavy chain;
3) A first polyA signal sequence;
4) A second promoter;
5) A second polynucleotide encoding an antibody light chain;
6) A second polyA signal sequence;
7) A third promoter;
8) A third polynucleotide encoding an Fc fusion protein or a scFv-Fc fusion protein;
9) A modified IRES as described herein, e.g., of any one of SEQ ID NOs: 3-4, 9-25 and 33-42, e.g., of any one of SEQ ID NOs: 12-14, 17, 22, 24, 40 and 42;
10) A selection marker, e.g., a polynucleotide encoding a GS or a polynucleotide encoding an antibiotic resistance gene; and
11) A third polyA signal sequence.

In some embodiments, the set of expression cassettes comprises from 5′ to 3′ the following operably linked elements:
1) A first promoter;
2) A first polynucleotide encoding an antibody heavy chain;
3) A first polyA signal sequence;
4) A second promoter;
5) A second polynucleotide encoding an antibody light chain;
6) A first modified IRES as described herein, e.g., of any one of SEQ ID NOs: 3-4, 9-25 and 33-42, e.g., of any one of SEQ ID NOs: 12-14, 17, 22, 24, 40 and 42;
7) A first selection marker, e.g., a polynucleotide encoding a GS or a polynucleotide encoding an antibiotic resistance gene;
8) A second polyA signal sequence;
9) A third promoter;
10) A third polynucleotide encoding an Fc fusion protein or a scFv-Fc fusion protein;
11) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
12) A second selection marker, e.g., a polynucleotide encoding a GS or a polynucleotide encoding an antibiotic resistance gene, optionally wherein the second selection marker is different from the first selection marker; and
13) A third polyA signal sequence.

In some embodiments, the set of expression cassettes comprises from 5′ to 3′ the following operably linked elements:
1) A first promoter;
2) A first polynucleotide encoding an antibody light chain;
3) A modified IRES as described herein, e.g., of any one of SEQ ID NOs: 3-4, 9-25 and 33-42, e.g., of any one of SEQ ID NOs: 12-14, 17, 22, 24, 40 and 42;
4) A selection marker, e.g., a polynucleotide encoding a GS or a polynucleotide encoding an antibiotic resistance gene;
5) A first polyA signal sequence;
6) A second promoter;
7) A second polynucleotide encoding an antibody heavy chain;
8) A second polyA signal sequence;
9) A third promoter;
10) A third polynucleotide encoding an Fc fusion protein or a scFv-Fc fusion protein; and
11) A third polyA signal sequence.

In some embodiments, the set of expression cassettes comprises from 5′ to 3′ the following operably linked elements:
1) A first promoter;
2) A first polynucleotide encoding an antibody heavy chain;
3) A modified IRES as described herein, e.g., of any one of SEQ ID NOs: 3-4, 9-25 and 33-42, e.g., of any one of SEQ ID NOs: 12-14, 17, 22, 24, 40 and 42;
4) A selection marker, e.g., a polynucleotide encoding a GS or a polynucleotide encoding an antibiotic resistance gene;
5) A first polyA signal sequence;
6) A second promoter;
7) A second polynucleotide encoding an antibody light chain;
8) A second polyA signal sequence;
9) A third promoter;
10) A third polynucleotide encoding an Fc fusion protein or a scFv-Fc fusion protein; and
11) A third polyA signal sequence.

In some embodiments, the set of expression cassettes comprises from 5′ to 3′ the following operably linked elements:
1) A first promoter;
2) A first polynucleotide encoding an antibody light chain;
3) A first modified IRES as described herein, e.g., of any one of SEQ ID NOs: 3-4, 9-25 and 33-42, e.g., of any one of SEQ ID NOs: 12-14, 17, 22, 24, 40 and 42;
4) A first selection marker, e.g., a polynucleotide encoding a GS or a polynucleotide encoding an antibiotic resistance gene;
5) A first polyA signal sequence;
6) A second promoter;
7) A second polynucleotide encoding an antibody heavy chain;
8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
9) A second selection marker, optionally, wherein the second selection marker is different from the first selection marker;
10) A second polyA signal sequence;
11) A third promoter;
12) A third polynucleotide encoding an Fc fusion protein or a scFv-Fc fusion protein;
13) A third modified IRES, optionally comprising a polynucleotide of any one of SEQ ID NOs: 3-4, 9-25 and 33-42, e.g., of any one of SEQ ID NOs: 12-14, 17, 22, 24, 40 and 42;
14) A third selection marker, optionally wherein the third selection marker is different from the first selection marker and the second selection marker; and
15) A third polyA signal sequence.

In some embodiments, the set of expression cassettes comprises from 5′ to 3′ the following operably linked elements:
1) A first promoter;
2) A first polynucleotide encoding an antibody heavy chain;
3) A first modified IRES as described herein, e.g., of any one of SEQ ID NOs: 3-4, 9-25 and 33-42, e.g., of any one of SEQ ID NOs: 12-14, 17, 22, 24, 40 and 42;
4) A first selection marker, e.g., a polynucleotide encoding a GS or a polynucleotide encoding an antibiotic resistance gene;
5) A first polyA signal sequence;
6) A second promoter;
7) A second polynucleotide encoding an antibody light chain;
8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
9) A second selection marker, optionally, wherein the second selection marker is different from the first selection marker;
10) A second polyA signal sequence;
11) A third promoter;
12) A third polynucleotide encoding an Fc fusion protein or a scFv-Fc fusion protein;
13) A third modified IRES, optionally comprising a polynucleotide of any one of SEQ ID NOs: 3-4, 9-25 and 33-42, e.g., of any one of SEQ ID NOs: 12-14, 17, 22, 24, 40 and 42;
14) A third selection marker, optionally wherein the third selection marker is different from the first selection marker and the second selection marker; and
15) A third polyA signal sequence.

In various embodiments, two or more of the first promoter, the second promoter and the third promoter are the same. In some embodiments, two or more of the first promoter, the second promoter and the third promoter are different. In some embodiments, two or more of the first promoter, the second promoter and the third promoter induce equivalent expression levels of a 3′ protein of interest. In some embodiments, two or more of the first promoter, the second promoter and the third promoter induce different expression levels of a 3′ protein of interest.

In various embodiments, two or more of the first modified IRES, the second modified IRES and the third modified IRES are the same. In various embodiments, two or more of the first modified IRES, the second modified IRES and the third modified IRES are different.

Generally, the first selection marker, the second selection marker and the third selection marker are different. For example, in some embodiments, the first selection marker comprises a polynucleotide encoding a GS, the second selection marker comprises a polynucleotide encoding a first antibiotic resistance gene and the third selection marker comprises a polynucleotide encoding a second antibiotic resistance gene. In some embodiments, the first selection marker comprises a polynucleotide encoding a first antibiotic resistance gene, the second selection marker comprises a polynucleotide encoding a GS and the third selection marker comprises a polynucleotide encoding a second antibiotic resistance gene. In some embodiments, the first selection marker comprises a polynucleotide encoding a first antibiotic resistance gene, the second selection marker comprises a polynucleotide encoding a second antibiotic resistance gene and the third selection marker comprises a polynucleotide encoding a GS. In some embodiments, the selection marker is an antibiotic resistance protein encoded by NeoR, BsdR, HygR, PuroR, BleoR (a.k.a., ZeoR). In some embodiments, the selection marker is a polynucleotide encoding a glutamine synthetase (GS) enzyme. In some embodiments, GS is selected from a human GS, a fish GS, a koala GS, a hamster GS and variants thereof. In some embodiments, the GS is a variant human GS. In some embodiments the human variant GS is R324S (SEQ ID NO:30).

In various embodiments, two or more of the first polyA signal sequence, the second polyA signal sequence and the third polyA signal sequence are the same. In various embodiments, two or more of the first polyA signal sequence, the second polyA signal sequence and the third polyA signal sequence are different.

In various embodiments, the first expression cassette containing the first polynucleotide, the second expression cassette containing the second polynucleotide and the third expression cassette containing the third polynucleotide are in one vector (e.g., a tricistronic vector). In various embodiments, the first expression cassette containing the first polynucleotide, the second expression cassette containing the second polynucleotide and the third expression cassette containing the third polynucleotide are in two vectors (e.g., a bicistronic vector and a monocistronic vector). In various embodiments, the first expression cassette containing the first polynucleotide, the second expression cassette containing the second polynucleotide and the third expression cassette containing the third polynucleotide are in three vectors (e.g., three monocistronic vectors).

d. Biologic Molecule Having Four Polypeptide Chains

Further provided are sets of four or more expression cassettes comprising elements for expressing a protein of interest (i.e., a biologic molecule) comprising four polypeptide chains. A set of four or more expression cassettes can be in one or more vectors (e.g., a multi-cistronic, e.g., a bicistronic, a tricistronic vector or a vector comprising four cistrons). Illustrative biologic molecules expressed from expression cassettes having coding sequences for three polypeptide chains include without limitation, Fab-Fc x Fab-Fc heterodimers, including traditional and CrossMab formats. Biologic molecules having four polypeptide chains can have first and second immunoglobulin heavy chains and first and second immunoglobulin light chains.

In some embodiments, the set of expression cassettes comprises from 5′ to 3′ the following operably linked elements:
1) A first promoter;
2) A first polynucleotide encoding a first polypeptide of the protein of interest;
3) A first polyA signal sequence;
4) A second promoter;
5) A second polynucleotide encoding a second polypeptide of the protein of interest;
6) A second polyA signal sequence;
7) A third promoter;
8) A third polynucleotide encoding a third polypeptide of the protein of interest;
9) A third polyA signal sequence;
10) A fourth promoter;
11) A fourth polynucleotide encoding a fourth polypeptide of the protein of interest;
12) A modified IRES as described herein, e.g., of any one of SEQ ID NOs: 3-4, 9-25 and 33-42, e.g., of any one of SEQ ID NOs: 12-14, 17, 22, 24, 40 and 42;
13) A selection marker, e.g., a polynucleotide encoding a GS or a polynucleotide encoding an antibiotic resistance gene; and
14) A fourth polyA signal sequence.

In some embodiments, the set of expression cassettes comprises from 5′ to 3′ the following operably linked elements:
1) A first promoter;
2) A first polynucleotide encoding a first polypeptide of the protein of interest;
3) A modified IRES as described herein, e.g., of any one of SEQ ID NOs: 3-4, 9-25 and 33-42, e.g., of any one of SEQ ID NOs: 12-14, 17, 22, 24, 40 and 42;
4) A selection marker, e.g., a polynucleotide encoding a GS or a polynucleotide encoding an antibiotic resistance gene;
5) A first polyA signal sequence;
6) A second promoter;
7) A second polynucleotide encoding a second polypeptide of the protein of interest;
8) A second polyA signal sequence;
9) A third promoter;
10) A third polynucleotide encoding a third polypeptide of the protein of interest;
11) A third polyA signal sequence;
12) A fourth promoter;
13) A fourth polynucleotide encoding a fourth polypeptide of the protein of interest; and
14) A fourth polyA signal sequence.

In some embodiments, the set of expression cassettes comprises from 5′ to 3′ the following operably linked elements:
1) A first promoter;
2) A first polynucleotide encoding a first polypeptide of the protein of interest;
3) A first modified IRES as described herein, e.g., of any one of SEQ ID NOs: 3-4, 9-25 and 33-42, e.g., of any one of SEQ ID NOs: 12-14, 17, 22, 24, 40 and 42;
4) A first selection marker, e.g., a polynucleotide encoding a GS or a polynucleotide encoding an antibiotic resistance gene;
5) A first polyA signal sequence;
6) A second promoter;
7) A second polynucleotide encoding a second polypeptide of the protein of interest;
8) A second polyA signal sequence;
9) A third promoter;
10) A third polynucleotide encoding a third polypeptide of the protein of interest;
11) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
12) A second selection marker, e.g., a polynucleotide encoding a GS, e.g., human variant GS is R324S (SEQ ID NO:30) or an antibiotic resistance gene, e.g., encoded by BleoR, optionally, wherein the second selection marker is different from the first selection marker;
13) A third polyA signal sequence;
14) A fourth promoter;
15) A fourth polynucleotide encoding a fourth polypeptide of the protein of interest; and
16) A fourth polyA signal sequence.

In some embodiments, the set of expression cassettes comprises from 5′ to 3′ the following operably linked elements:
1) A first promoter;
2) A first polynucleotide encoding a first polypeptide of the protein of interest;
3) A first polyA signal sequence;
4) A second promoter;
5) A second polynucleotide encoding a second polypeptide of the protein of interest;
6) A modified IRES as described herein, e.g., of any one of SEQ ID NOs: 3-4, 9-25 and 33-42, e.g., of any one of SEQ ID NOs: 12-14, 17, 22, 24, 40 and 42;
7) A selection marker, e.g., a polynucleotide encoding a GS or a polynucleotide encoding an antibiotic resistance gene;
8) A second polyA signal sequence;
9) A third promoter;
10) A third polynucleotide encoding a third polypeptide of the protein of interest;
11) A third polyA signal sequence;
12) A fourth promoter;
13) A fourth polynucleotide encoding a fourth polypeptide of the protein of interest; and
14) A fourth polyA signal sequence.

In some embodiments, the set of expression cassettes comprises from 5′ to 3′ the following operably linked elements:
1) A first promoter;
2) A first polynucleotide encoding a first polypeptide of the protein of interest;
3) A first polyA signal sequence;
4) A second promoter;
5) A second polynucleotide encoding a second polypeptide of the protein of interest;
6) A first modified IRES as described herein, e.g., of any one of SEQ ID NOs: 3-4, 9-25 and 33-42, e.g., of any one of SEQ ID NOs: 12-14, 17, 22, 24, 40 and 42;
7) A selection marker, e.g., a polynucleotide encoding a GS or a polynucleotide encoding an antibiotic resistance gene;
8) A second polyA signal sequence;
9) A third promoter;
10) A third polynucleotide encoding a third polypeptide of the protein of interest;
11) A third polyA signal sequence;
12) A fourth promoter;
13) A fourth polynucleotide encoding a fourth polypeptide of the protein of interest;
14) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
15) A second selection marker, e.g., a polynucleotide encoding a GS, e.g., human variant GS is R324S (SEQ ID NO:30) or an antibiotic resistance gene, e.g., encoded by BleoR, optionally, wherein the second selection marker is different from the first selection marker; and
16) A fourth polyA signal sequence.

In some embodiments, the set of expression cassettes comprises from 5′ to 3′ the following operably linked elements:
1) A first promoter;
2) A first polynucleotide encoding a first polypeptide of the protein of interest;
3) A first polyA signal sequence;
4) A second promoter;
5) A second polynucleotide encoding a second polypeptide of the protein of interest;
6) A second polyA signal sequence;
7) A third promoter;
8) A third polynucleotide encoding a third polypeptide of the protein of interest;
9) A modified IRES as described herein, e.g., of any one of SEQ ID NOs: 3-4, 9-25 and 33-42, e.g., of any one of SEQ ID NOs: 12-14, 17, 22, 24, 40 and 42;
10) A selection marker, e.g., a polynucleotide encoding a GS or a polynucleotide encoding an antibiotic resistance gene;
11) A third polyA signal sequence;
12) A fourth promoter;
13) A fourth polynucleotide encoding a fourth polypeptide of the protein of interest; and
14) A fourth polyA signal sequence.

In some embodiments, the set of expression cassettes comprises from 5′ to 3′ the following operably linked elements:
1) A first promoter;
2) A first polynucleotide encoding a first polypeptide of the protein of interest;
3) A first modified IRES as described herein, e.g., of any one of SEQ ID NOs: 3-4, 9-25 and 33-42, e.g., of any one of SEQ ID NOs: 12-14, 17, 22, 24, 40 and 42;
4) A first selection marker, e.g., a polynucleotide encoding a GS or a polynucleotide encoding an antibiotic resistance gene;
5) A first polyA signal sequence;
6) A second promoter;
7) A second polynucleotide encoding a second polypeptide of the protein of interest;
8) A second polyA signal sequence;
9) A third promoter;
10) A third polynucleotide encoding a third polypeptide of the protein of interest;
11) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
12) A second selection marker, e.g., a polynucleotide encoding a GS, e.g., human variant GS is R324S (SEQ ID NO:30) or an antibiotic resistance gene, e.g., encoded by BleoR, optionally, wherein the second selection marker is different from the first selection marker;
13) A third polyA signal sequence;
14) A fourth promoter;
15) A fourth polynucleotide encoding a fourth polypeptide of the protein of interest; and
16) A fourth polyA signal sequence.

In some embodiments, the set of expression cassettes comprises from 5′ to 3′ the following operably linked elements:
1) A first promoter;
2) A first polynucleotide encoding a first polypeptide of the protein of interest;
3) A first modified IRES as described herein, e.g., of any one of SEQ ID NOs: 3-4, 9-25 and 33-42, e.g., of any one of SEQ ID NOs: 12-14, 17, 22, 24, 40 and 42;
4) A first selection marker, e.g., a polynucleotide encoding a GS or a polynucleotide encoding an antibiotic resistance gene;
5) A first polyA signal sequence;
6) A second promoter;
7) A second polynucleotide encoding a second polypeptide of the protein of interest;
8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
9) A second selection marker, e.g., a polynucleotide encoding a GS, e.g., human variant GS is R324S (SEQ ID NO:30) or an antibiotic resistance gene, e.g., encoded by BleoR, optionally, wherein the second selection marker is different from the first selection marker;
10) A second polyA signal sequence;
11) A third promoter;
12) A third polynucleotide encoding a third polypeptide of the protein of interest;
13) A third modified IRES, optionally comprising a polynucleotide of any one of SEQ ID NOs: 3-4, 9-25 and 33-42, e.g., of any one of SEQ ID NOs: 12-14, 17, 22, 24, 40 and 42;
14) A third selection marker, e.g., a polynucleotide encoding a GS, e.g., human variant GS is R324S (SEQ ID NO:30) or an antibiotic resistance gene, e.g., encoded by BleoR, optionally wherein the third selection marker is different from the first selection marker and the second selection marker;
15) A third polyA signal sequence;
16) A fourth promoter;
17) A fourth polynucleotide encoding a fourth polypeptide of the protein of interest;
18) A fourth modified IRES, optionally comprising a polynucleotide of any one of SEQ ID NOs: 3-4, 9-25 and 33-42, e.g., of any one of SEQ ID NOs: 12-14, 17, 22, 24, 40 and 42;
19) A fourth selection marker, e.g., a polynucleotide encoding a GS, e.g., human variant GS is R324S (SEQ ID NO:30) or an antibiotic resistance gene, e.g., encoded by BleoR, optionally wherein the fourth selection marker is different from the first selection marker, the second selection marker and the third selection marker; and
20) A fourth polyA signal sequence.

In some embodiments, the set of expression cassettes comprises from 5′ to 3′ the following operably linked elements:
1) A first promoter;
2) A first polynucleotide encoding a first antibody light chain;
3) A first polyA signal sequence;
4) A second promoter;
5) A second polynucleotide encoding a first antibody heavy chain;
6) A first modified IRES as described herein, e.g., of any one of SEQ ID NOs: 3-4, 9-25 and 33-42, e.g., of any one of SEQ ID NOs: 12-14, 17, 22, 24, 40 and 42;
7) A first selection marker, e.g., a polynucleotide encoding a GS or a polynucleotide encoding an antibiotic resistance gene;
8) A second polyA signal sequence;
9) A third promoter;
10) A third polynucleotide encoding a second antibody light chain;
11) A third polyA signal sequence;
12) A fourth promoter;
13) A fourth polynucleotide encoding a second antibody heavy chain;
14) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
15) A second selection marker, optionally, wherein the second selection marker is different from the first selection marker; and
16) A fourth polyA signal sequence. In some embodiments, the first antibody light chain pairs with the first antibody heavy chain to form an antigen binding domain. In some embodiments, the first antibody light chain pairs with the second antibody heavy chain to form an antigen binding domain. In some embodiments, the second antibody light chain pairs with the first antibody heavy chain to form an antigen binding domain. In some embodiments, the second antibody light chain pairs with the second antibody heavy chain to form an antigen binding domain.

In some embodiments, the set of expression cassettes comprises from 5′ to 3′ the following operably linked elements:
1) A first promoter;
2) A first polynucleotide encoding a light chain;
3) A first modified IRES as described herein, e.g., of any one of SEQ ID NOs: 3-4, 9-25 and 33-42, e.g., of any one of SEQ ID NOs: 12-14, 17, 22, 24, 40 and 42;
4) A first selection marker, e.g., a polynucleotide encoding a GS or a polynucleotide encoding an antibiotic resistance gene;
5) A first polyA signal sequence;
6) A second promoter;
7) A second polynucleotide encoding a first antibody heavy chain;
8) A second polyA signal sequence;
9) A third promoter;
10) A third polynucleotide encoding a second antibody light chain;
11) A second modified IRES, optionally comprising a polynucleotide of any of SEQ ID NOs: 3-4, 9-25 and 33-42, e.g., of any one of SEQ ID NOs: 12-14, 17, 22, 24, 40 and 42;
12) A second selection marker, e.g., a polynucleotide encoding a GS, e.g., human variant GS is R324S (SEQ ID NO:30) or an antibiotic resistance gene, e.g., encoded by BleoR, optionally, wherein the second selection marker is different from the first selection marker; and
13) A third polyA signal sequence;
14) A fourth promoter;
15) A fourth polynucleotide encoding a second antibody heavy chain; and
16) A fourth polyA signal sequence. In some embodiments, the first antibody light chain pairs with the first antibody heavy chain to form an antigen binding domain. In some embodiments, the first antibody light chain pairs with the second antibody heavy chain to form an antigen binding domain. In some embodiments, the second antibody light chain pairs with the first antibody heavy chain to form an antigen binding domain. In some embodiments, the second antibody light chain pairs with the second antibody heavy chain to form an antigen binding domain.

In some embodiments, the set of expression cassettes comprises from 5′ to 3′ the following operably linked elements:
1) A first promoter;
2) A first polynucleotide encoding a first antibody light chain;
3) A first modified IRES as described herein, e.g., of any one of SEQ ID NOs: 3-4, 9-25 and 33-42, e.g., of any one of SEQ ID NOs: 12-14, 17, 22, 24, 40 and 42;
4) A first selection marker, e.g., a polynucleotide encoding a GS or a polynucleotide encoding an antibiotic resistance gene;
5) A first polyA signal sequence;
6) A second promoter;
7) A second polynucleotide encoding a first antibody heavy chain;
8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
9) A second selection marker, e.g., a polynucleotide encoding a GS, e.g., human variant GS is R324S (SEQ ID NO:30) or an antibiotic resistance gene, e.g., encoded by BleoR, optionally, wherein the second selection marker is different from the first selection marker;
10) A second polyA signal sequence;
11) A third promoter;
12) A third polynucleotide encoding a second antibody light chain;
13) A third modified IRES, optionally comprising a polynucleotide of any one of SEQ ID NOs: 3-4, 9-25 and 33-42, e.g., of any one of SEQ ID NOs: 12-14, 17, 22, 24, 40 and 42;
14) A third selection marker, e.g., a polynucleotide encoding a GS, e.g., human variant GS is R324S (SEQ ID NO:30) or an antibiotic resistance gene, e.g., encoded by BleoR, optionally wherein the third selection marker is different from the first selection marker and the second selection marker;
15) A third polyA signal sequence;
16) A fourth promoter;
17) A fourth polynucleotide encoding a second antibody heavy chain;
18) A fourth modified IRES, optionally comprising a polynucleotide of any one of SEQ ID NOs: 3-4, 9-25 and 33-42, e.g., of any one of SEQ ID NOs: 12-14, 17, 22, 24, 40 and 42;
19) A fourth selection marker, e.g., a polynucleotide encoding a GS, e.g., human variant GS is R324S (SEQ ID NO:30) or an antibiotic resistance gene, e.g., encoded by BleoR, optionally wherein the fourth selection marker is different from the first selection marker, the second selection marker and the third selection marker; and
20) A fourth polyA signal sequence. In some embodiments, the first antibody light chain pairs with the first antibody heavy chain to form an antigen binding domain. In some embodiments, the first antibody light chain pairs with the second antibody heavy chain to form an antigen binding domain. In some embodiments, the second antibody light chain pairs with the first antibody heavy chain to form an antigen binding domain. In some embodiments, the second antibody light chain pairs with the second antibody heavy chain to form an antigen binding domain.

In some embodiments, the set of expression cassettes comprises from 5′ to 3′ the following operably linked elements:
1) A first promoter;
2) A first polynucleotide encoding a first antibody heavy chain;
3) A first polyA signal sequence;
4) A second promoter;
5) A second polynucleotide encoding a first antibody light chain;
6) A first modified IRES as described herein, e.g., of any one of SEQ ID NOs: 3-4, 9-25 and 33-42, e.g., of any one of SEQ ID NOs: 12-14, 17, 22, 24, 40 and 42;
7) A first selection marker, e.g., a polynucleotide encoding a GS or a polynucleotide encoding an antibiotic resistance gene;
8) A second polyA signal sequence;
9) A third promoter;
10) A third polynucleotide encoding a second antibody heavy chain;
11) A third polyA signal sequence;
12) A fourth promoter;
13) A fourth polynucleotide encoding a second antibody light chain;
14) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
15) A second selection marker, e.g., a polynucleotide encoding a GS, e.g., human variant GS is R324S (SEQ ID NO:30) or an antibiotic resistance gene, e.g., encoded by BleoR, optionally, wherein the second selection marker is different from the first selection marker; and
16) A fourth polyA signal sequence. In some embodiments, the first antibody light chain pairs with the first antibody heavy chain to form an antigen binding domain. In some embodiments, the first antibody light chain pairs with the second antibody heavy chain to form an antigen binding domain. In some embodiments, the second antibody light chain pairs with the first antibody heavy chain to form an antigen binding domain. In some embodiments, the second antibody light chain pairs with the second antibody heavy chain to form an antigen binding domain.

In some embodiments, the set of expression cassettes comprises from 5′ to 3′ the following operably linked elements:
1) A first promoter;
2) A first polynucleotide encoding a first antibody heavy chain;
3) A first modified IRES as described herein, e.g., of any one of SEQ ID NOs: 3-4, 9-25 and 33-42, e.g., of any one of SEQ ID NOs: 12-14, 17, 22, 24, 40 and 42;
4) A first selection marker, e.g., a polynucleotide encoding a GS or a polynucleotide encoding an antibiotic resistance gene;
5) A first polyA signal sequence;
6) A second promoter;
7) A second polynucleotide encoding a first antibody light chain;
8) A second polyA signal sequence;
9) A third promoter;
10) A third polynucleotide encoding a second antibody heavy chain;
11) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
12) A second selection marker, optionally, wherein the second selection marker is different from the first selection marker; and
13) A third polyA signal sequence;
14) A fourth promoter;
15) A fourth polynucleotide encoding a second antibody light chain; and
16) A fourth polyA signal sequence. In some embodiments, the first antibody light chain pairs with the first antibody heavy chain to form an antigen binding domain. In some embodiments, the first antibody light chain pairs with the second antibody heavy chain to form an antigen binding domain. In some embodiments, the second antibody light chain pairs with the first antibody heavy chain to form an antigen binding domain. In some embodiments, the second antibody light chain pairs with the second antibody heavy chain to form an antigen binding domain.

In some embodiments, the set of expression cassettes comprises from 5′ to 3′ the following operably linked elements:
1) A first promoter;
2) A first polynucleotide encoding a first antibody heavy chain;
3) A first modified IRES as described herein, e.g., of any one of SEQ ID NOs: 3-4, 9-25 and 33-42, e.g., of any one of SEQ ID NOs: 12-14, 17, 22, 24, 40 and 42;
4) A first selection marker, e.g., a polynucleotide encoding a GS or a polynucleotide encoding an antibiotic resistance gene;
5) A first polyA signal sequence;
6) A second promoter;
7) A second polynucleotide encoding a first antibody light chain;
8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
9) A second selection marker, e.g., a polynucleotide encoding a GS, e.g., human variant GS is R324S (SEQ ID NO:30) or an antibiotic resistance gene, e.g., encoded by BleoR, optionally, wherein the second selection marker is different from the first selection marker;
10) A second polyA signal sequence;
11) A third promoter;
12) A third polynucleotide encoding a second antibody heavy chain;
13) A third modified IRES, optionally comprising a polynucleotide of any one of SEQ ID NOs: 3-4, 9-25 and 33-42, e.g., of any one of SEQ ID NOs: 12-14, 17, 22, 24, 40 and 42;
14) A third selection marker, wherein the third selection marker is different from the first selection marker and the second selection marker; and
15) A third polyA signal sequence;
16) A fourth promoter;
17) A fourth polynucleotide encoding a second antibody light chain;
18) A fourth modified IRES, optionally comprising a polynucleotide of any one of SEQ ID NOs: 3-4, 9-25 and 33-42, e.g., of any one of SEQ ID NOs: 12-14, 17, 22, 24, 40 and 42;
19) A fourth selection marker, wherein the fourth selection marker is different from the first selection marker, the second selection marker and the third selection marker; and
20) A fourth polyA signal sequence. In some embodiments, the first antibody light chain pairs with the first antibody heavy chain to form an antigen binding domain. In some embodiments, the first antibody light chain pairs with the second antibody heavy chain to form an antigen binding domain. In some embodiments, the second antibody light chain pairs with the first antibody heavy chain to form an antigen binding domain. In some embodiments, the second antibody light chain pairs with the second antibody heavy chain to form an antigen binding domain.

In various embodiments, two or more (i.e., at least two) of the first promoter, the second promoter, the third promoter and the fourth promoter are the same. In some embodiments, two or more of the first promoter, the second promoter, the third promoter and the fourth promoter are different. In some embodiments, two or more of the first promoter, the second promoter, the third promoter and the fourth promoter induce equivalent expression levels of a 3′ protein of interest. In some embodiments, the first promoter, the second promoter, the third promoter and the fourth promoter induce different expression levels of a 3′ protein of interest.

In various embodiments, two or more of the first modified IRES, the second modified IRES, the third modified IRES and the fourth modified IRES are the same. In various embodiments, two or more of the first modified IRES, the second modified IRES, the third modified IRES and the fourth modified IRES are different.

In some embodiments, the first selection marker, the second selection marker, the third selection marker and the fourth selection marker are different. For example, in some embodiments, the first selection marker comprises a polynucleotide encoding a GS, the second selection marker comprises a polynucleotide encoding a first antibiotic resistance gene, the third selection marker comprises a polynucleotide encoding a second antibiotic resistance gene and the fourth selection marker comprises a polynucleotide encoding a third antibiotic resistance gene. In some embodiments, the first selection marker comprises a polynucleotide encoding a first antibiotic resistance gene, the second selection marker comprises a polynucleotide encoding a GS, the third selection marker comprises a polynucleotide encoding a second antibiotic resistance gene and the fourth selection marker comprises a polynucleotide encoding a third antibiotic resistance gene. In some embodiments, the first selection marker comprises a polynucleotide encoding a first antibiotic resistance gene, the second selection marker comprises a polynucleotide encoding a second antibiotic resistance gene, the third selection marker comprises a polynucleotide encoding a GS, and the fourth selection marker comprises a polynucleotide encoding a third antibiotic resistance gene. In some embodiments, the first selection marker comprises a polynucleotide encoding a first antibiotic resistance gene, the second selection marker comprises a polynucleotide encoding a second antibiotic resistance gene, the third selection marker comprises a polynucleotide encoding a third antibiotic resistance gene, and the fourth selection marker comprises a polynucleotide encoding a GS. In some embodiments, two or more of the selection markers are the same. In some embodiments, the selection marker is an antibiotic resistance protein encoded by NeoR, BsdR, HygR, PuroR, BleoR (a.k.a., ZeoR). In some embodiments, the selection marker is a polynucleotide encoding a glutamine synthetase (GS) enzyme. In some embodiments, GS is selected from a human GS, a fish GS, a koala GS, a hamster GS and variants thereof. In some embodiments, the GS is a variant human GS. In some embodiments the human variant GS is R324S (SEQ ID NO:30).

In various embodiments, two or more of the first polyA signal sequence, the second polyA signal sequence, the third polyA signal sequence and the fourth polyA signal sequence are the same. In various embodiments, two or more of the first polyA signal sequence, the second polyA signal sequence, the third polyA signal sequence and the fourth polyA signal sequence are different.

In various embodiments, the first expression cassette containing the first polynucleotide, the second expression cassette containing the second polynucleotide, the third expression cassette containing the third polynucleotide, and the fourth expression cassette containing the fourth polynucleotide are in one vector. In various embodiments, the first expression cassette containing the first polynucleotide, the second expression cassette containing the second polynucleotide, the third expression cassette containing the third polynucleotide, and the fourth expression cassette containing the fourth polynucleotide are in two vectors. In various embodiments, the first expression cassette containing the first polynucleotide, the second expression cassette containing the second polynucleotide, the third expression cassette containing the third polynucleotide, and the fourth expression cassette containing the fourth polynucleotide are in three vectors. In various embodiments, the first expression cassette containing the first polynucleotide, the second expression cassette containing the second polynucleotide, the third expression cassette containing the third polynucleotide, and the fourth expression cassette containing the fourth polynucleotide are in four vectors.

### 4. Vectors

Further provided are vectors comprising one or more polynucleotides encoding one or more of the proteins of interest (e.g., antibodies, Fc fusion proteins, multi-specific binding molecules), described herein. Also provided are vectors comprising one or more expression cassettes (e.g., multi-cistronic, bicistronic, tricistronic) for expressing one or more of the proteins of interest (e.g., antibodies, Fc fusion proteins, multi-specific binding molecules), described herein. Moreover, expression cassettes for expressing polypeptide chains of a biologic molecule comprised of two or more polypeptide chains, e.g., two, three or four polypeptide chains, can be expressed from one or multiple expression vectors, e.g., one, two, three or four vectors. A vector can be of any type, for example, a recombinant vector such as an expression vector. Vectors include, but are not limited to, plasmids, cosmids, bacterial artificial chromosomes (BAC) and yeast artificial chromosomes (YAC) and vectors derived from bacteriophages or plant or animal (including human) viruses. Vectors can comprise an origin of replication and one or more selection markers recognized by and operational in the proposed host cell (e.g., including prokaryotic and eukaryotic host cells), and in the case of expression vectors, promoter, terminator, and other regulatory regions recognized by the host cell. In additional embodiments, a vector comprises an expression cassette as described herein, e.g., comprising a polynucleotide encoding a protein of interest of the disclosure operably linked to a promoter and optionally additional regulatory elements. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome. Vectors include, but are not limited to, those suitable for recombinant production of the antibodies disclosed herein. The term “vector,” as used herein, refers to a nucleic acid molecule capable of propagating another nucleic acid. The term includes the vector as a self-replicating nucleic acid structure as well as the vector incorporated into the genome of a host cell into which it has been introduced. Some vectors are suitable for delivering the nucleic acid molecule or polynucleotide of the present application. Certain vectors are capable of directing the expression of nucleic acids to which they are operatively linked. Such vectors are referred to herein as expression vectors.

The choice of the vector is dependent on the recombinant procedures followed and the host used. Introduction of vectors into host cells can be effected by inter alia calcium phosphate transfection, virus infection, DEAE-dextran-mediated transfection, lipofectamine transfection or electroporation. Vectors may be autonomously replicating or may replicate together with the chromosome into which they have been integrated. In certain embodiments, the vectors contain one or more selection markers. The choice of the markers may depend on the host cells of choice. These include, but are not limited to, kanamycin, neomycin, puromycin, hygromycin, zeocin, ampicillin (AmpR), thymidine kinase gene from Herpes simplex virus (HSV-TK), glutamine synthetase (GS) and dihydrofolate reductase (DHFR) gene. Vectors comprising one or more polynucleotides encoding the antibodies described herein, operably linked to one or more polynucleotides encoding proteins or peptides that can be used to isolate the polypeptides of interest, are also covered by the disclosure. These proteins or peptides include, but are not limited to, glutathione-S-transferase, maltose binding protein, metal-binding polyhistidine, green fluorescent protein, luciferase and beta-galactosidase. In other embodiments, the vector that is used is or is based on pcDNA™3.1+(ThermoFisher, MA), pCI (Promega, WI), pIRES (Takara, CA), or pCGS3 (Sigma-Aldrich, MO).

### Multiple Vectors for Expressing Biologic Molecules Having Two Polypeptide Chains

In some embodiments, provided is a set of vectors comprising a first vector and a second vector, the first vector comprising:
(a) a first expression cassette comprising from 5′ to 3′ the following operably linked elements:
   1) A first promoter;
   2) A first polynucleotide encoding a first polypeptide of a protein of interest;
   3) A first modified IRES as described herein, e.g., of any one of SEQ ID NOs: 3-4, 9-25 and 33-42, e.g., of any one of SEQ ID NOs: 12-14, 17, 22, 24, 40 and 42;
   4) A first selection marker, e.g., a polynucleotide encoding a GS, e.g., human variant GS is R324S (SEQ ID NO:30) or an antibiotic resistance gene, e.g., encoded by BleoR; and
   5) A first polyA signal sequence; and
(b) a second vector, the second vector comprising a second expression cassette comprising from 5′ to 3′ the following operably linked elements:
   1) A second promoter;
   2) A second polynucleotide encoding a second polypeptide of a protein of interest;
   3) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
   4) A second selection marker, e.g., a polynucleotide encoding a GS, e.g., human variant GS is R324S (SEQ ID NO:30) or an antibiotic resistance gene, e.g., encoded by BleoR, optionally wherein the second selection marker is different from the first selection marker; and
   5) A second polyA signal sequence.

### Multiple Vectors for Expressing Biologic Molecules Having Three Polypeptide Chains

In some embodiments, provided is a set of vectors comprising a first vector and a second vector, the first vector comprising:
(a) a first expression cassette comprising from 5′ to 3′ the following operably linked elements:
   1) A first promoter;
   2) A first polynucleotide encoding a first polypeptide of the protein of interest;
   3) A first polyA signal sequence;
   4) A second promoter;
   5) A second polynucleotide encoding a second polypeptide of the protein of interest;
   6) A first modified IRES as described herein, e.g., of any one of SEQ ID NOs: 3-4, 9-25 and 33-42, e.g., of any one of SEQ ID NOs: 12-14, 17, 22, 24, 40 and 42;
   7) A first selection marker, e.g., a polynucleotide encoding a GS, e.g., human variant GS is R324S (SEQ ID NO:30) or an antibiotic resistance gene, e.g., encoded by BleoR; and
   8) A second polyA signal sequence; and
(b) a second vector, the second vector comprising a second expression cassette comprising from 5′ to 3′ the following operably linked elements:
   1) A third promoter;
   2) A third polynucleotide encoding a third polypeptide of a protein of interest;
   3) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
   4) A second selection marker, e.g., a polynucleotide encoding a GS, e.g., human variant GS is R324S (SEQ ID NO:30) or an antibiotic resistance gene, e.g., encoded by BleoR, optionally wherein the second selection marker is different from the first selection marker; and
   5) A third polyA signal sequence.

In some embodiments, provided is a set of vectors comprising a first vector and a second vector, the first vector comprising:
(a) a first expression cassette comprising from 5′ to 3′ the following operably linked elements:
   1) A first promoter;
   2) A first polynucleotide encoding a first polypeptide of the protein of interest;
   3) A modified IRES as described herein, e.g., of any one of SEQ ID NOs: 3-4, 9-25 and 33-42, e.g., of any one of SEQ ID NOs: 12-14, 17, 22, 24, 40 and 42;
   4) A selection marker, e.g., a polynucleotide encoding a GS, e.g., human variant GS is R324S (SEQ ID NO:30) or an antibiotic resistance gene, e.g., encoded by BleoR;
   5) A first polyA signal sequence;
   6) A second promoter;
   7) A second polynucleotide encoding a second polypeptide of the protein of interest; and
   8) A second polyA signal sequence; and
(b) a second vector, the second vector comprising a second expression cassette comprising from 5′ to 3′ the following operably linked elements:
   1) A third promoter;
   2) A third polynucleotide encoding a third polypeptide of a protein of interest;
   3) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
   4) A second selection marker, e.g., a polynucleotide encoding a GS, e.g., human variant GS is R324S (SEQ ID NO:30) or an antibiotic resistance gene, e.g., encoded by BleoR, optionally wherein the second selection marker is different from the first selection marker; and
   5) A third polyA signal sequence.

In some embodiments, provided is a set of vectors comprising a first vector and a second vector, the first vector comprising:
(a) a first expression cassette comprising from 5′ to 3′ the following operably linked elements:
   1) A first promoter;
   2) A first polynucleotide encoding a first polypeptide of the protein of interest;
   3) A first modified IRES as described herein, e.g., of any one of SEQ ID NOs: 3-4, 9-25 and 33-42, e.g., of any one of SEQ ID NOs: 12-14, 17, 22, 24, 40 and 42;
   4) A first selection marker, e.g., a polynucleotide encoding a GS, e.g., human variant GS is R324S (SEQ ID NO:30) or an antibiotic resistance gene, e.g., encoded by BleoR;
   5) A first polyA signal sequence;
   6) A second promoter;
   7) A second polynucleotide encoding a second polypeptide of the protein of interest;
   8) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
   9) A second selection marker, e.g., a polynucleotide encoding a GS, e.g., human variant GS is R324S (SEQ ID NO:30) or an antibiotic resistance gene, e.g., encoded by BleoR, e.g., optionally, wherein the second selection marker is different from the first selection marker;
   10) A second polyA signal sequence; and
(b) a second vector, the second vector comprising a second expression cassette comprising from 5′ to 3′ the following operably linked elements:
   1) A third promoter;
   2) A third polynucleotide encoding a third polypeptide of a protein of interest;
   3) A third modified IRES, optionally as described herein, e.g., of any one of SEQ ID NOs: 3-4, 9-25 and 33-42, e.g., of any one of SEQ ID NOs: 12-14, 17, 22, 24, 40 and 42;
   4) A third selection marker, e.g., a polynucleotide encoding a GS, e.g., human variant GS is R324S (SEQ ID NO:30) or an antibiotic resistance gene, e.g., encoded by BleoR or ZeoR, optionally wherein the third selection marker is different from the first selection marker and the second selection marker; and
   5) A third polyA signal sequence.

In some embodiments, provided is a set of vectors comprising a first vector, a second vector and a third vector, the first vector comprising:
(a) a first expression cassette comprising from 5′ to 3′ the following operably linked elements:
   1) A first promoter;
   2) A first polynucleotide encoding a first polypeptide of the protein of interest;
   3) A first modified IRES as described herein, e.g., of any one of SEQ ID NOs: 3-4, 9-25 and 33-42, e.g., of any one of SEQ ID NOs: 12-14, 17, 22, 24, 40 and 42;
   4) A first selection marker, e.g., a polynucleotide encoding a GS, e.g., human variant GS is R324S (SEQ ID NO:30) or an antibiotic resistance gene, e.g., encoded by BleoR; and
   5) A first polyA signal sequence;
(b) a second vector, the second vector comprising a second expression cassette comprising from 5′ to 3′ the following operably linked elements:
   1) A second promoter;
   2) A second polynucleotide encoding a second polypeptide of the protein of interest;
   3) A second modified IRES, optionally comprising a polynucleotide of any one of SEQ ID NOs: 3-4, 9-25 and 33-42, e.g., of any one of SEQ ID NOs: 12-14, 17, 22, 24, 40 and 42;
   4) A second selection marker, e.g., a polynucleotide encoding a GS, e.g., human variant GS is R324S (SEQ ID NO:30) or an antibiotic resistance gene, e.g., encoded by BleoR; and
   5) A second polyA signal sequence; and
(b) a third vector, the second vector comprising a second expression cassette comprising from 5′ to 3′ the following operably linked elements:
   1) A third promoter;
   2) A third polynucleotide encoding a third polypeptide of a protein of interest;
   3) A third modified IRES, optionally as described herein, e.g., of any one of SEQ ID NOs: 3-4, 9-25 and 33-42, e.g., of any one of SEQ ID NOs: 12-14, 17, 22, 24, 40 and 42;
   4) A third selection marker, e.g., a polynucleotide encoding a GS, e.g., human variant GS is R324S (SEQ ID NO:30) or an antibiotic resistance gene, e.g., encoded by BleoR, optionally wherein the third selection marker is different from the first selection marker and the second selection marker; and
   5) A third polyA signal sequence.

### 5. Host Cells

The disclosure also provides host cells comprising one or more recombinant polynucleotides, one or more expression cassettes, or one or more vectors, as described herein. Any of a variety of host cells can be used. In one embodiment, a host cell is a eukaryotic cell, for example, a yeast cell, a plant cell, an insect cell, a mammalian cell. In some embodiments, the host cell is a mammalian cell such as a Chinese Hamster Ovary (CHO)-based or CHO-originated cell (e.g., CHO-S, CHO DG44, CHO DXB-11, ExpiCHO™, CHOZN® ZFN-modified GS−/− CHO cell line, CHOSOURCE™ GS knockout cell line, CHOK1SV, CHOK1SV GS-KO® cell line, CHO-K1, or other CHO-K1-derived cell lines), a COS cell, a BHK cell, a NS0 cell or a human cell. Examples of human host cells of use include without limitation, inter alia, HeLa, 911, AT1080, A549, HEK293, Expi293™ and HEK293T-cells.

The terms “host cell,” “host cell line,” and “host cell culture” are used interchangeably and refer to cells into which exogenous nucleic acid could be or has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with a polynucleotide encoding a protein of interest, as described herein. In some embodiments, the host cells are isolated.

### 6. Methods of Improving Expression of Protein of Interest

In various embodiments, the proteins of interest expressed from the expression cassettes comprising one or more modified IRES sequences, described herein, may be produced in eukaryotic cells, e.g., mammalian cells, e.g., CHO-based or CHO-originated cells. The proteins of interest can also be produced in eukaryotic cells such as transformed cell lines (e.g., 293E, 293T, Expi293™, COS, NIH3T3). In addition, the proteins of interest described herein can be expressed in a yeast cell such as Pichia (see, e.g., Powers et al., J Immunol Methods. 251:123-35 (2001)), Hanseula, or Saccharomyces. In one embodiment, the proteins of interest described herein are produced in a CHO-based or CHO-originated cell line (e.g., CHO-S, CHO DG44, CHO DXB-11, ExpiCHO™, CHOZN® ZFN-modified GS−/− CHO cell line, CHOSOURCE™ GS knockout cell line, CHOK1SV, CHOK1SV GS-KO® cell line, CHO-K1, or other CHO-K1-derived cell lines) or a HEK293 (e.g., Expi293™) cell line. To produce proteins of interest, one or more polynucleotides encoding the proteins of interest are constructed, introduced into an expression vector, and then expressed in one or more suitable host cells. Standard molecular biology techniques can be used to prepare the recombinant expression vector, transfect the host cells, select for transfectants, culture the transfected cells, and recover the proteins of interest (e.g. Fc fusion proteins, antibodies, multi-specific antigen binding molecules).

If the proteins of interest are to be expressed in mammalian host cells, e.g., such as CHO-based or CHO-originated cells, COS, and NIH3T3 cells, the expression vector includes a promoter useful for expression in these cells. In various embodiments, the promoter for expression of the proteins of interest in mammalian cells is a constitutive promoter or an inducible promoter. Illustrative promoters for expression of the proteins of interest in mammalian cells include without limitation those described above, and e.g., an SV40 promoter (Mulligan et al., Nature, 277:108 (1979)), a MMLV-LTR promoter, an EFlu promoter (Mizushima et al., Nucleic Acids Res., 18:5322 (1990)), or a CMV promoter. In addition to the nucleic acid sequence encoding the protein of interest thereof, the recombinant expression vectors may carry additional sequences, such as sequences that regulate replication of the vector in host cells (e.g., origins of replication) and selection marker genes. The selection marker gene facilitates selection of host cells into which the vector has been introduced (see e.g., U.S. Pat. Nos. 4,399,216, 4,634,665 and 5,179,017). In some embodiments, the selection marker gene confers resistance to drugs, such as G418, hygromycin, zeocin, blastcidin, puromycin, or methotrexate, on a host cell into which the vector has been introduced. Examples of vectors with selection markers include pMAM, pDR2, pBK-RSV, pBK-CMV, pOPRSV, pIRESbleo3, pIRESneo3, pIREShyg3, pIRESpuro3, and pOP13. In some embodiments, the selection marker is a glutamine synthetase (GS).

In one embodiment, the proteins of interest are produced in mammalian cells. Exemplary mammalian host cells for expressing the proteins of interest include Chinese Hamster Ovary (CHO cells) (including dhfr-CHO cells, e.g., described in Urlaub and Chasin (1980) Proc. Natl. Acad. Sci. USA 77: 4216-4220, used with a DHFR selection marker, e.g., as described in Kaufman and Sharp (1982) Mol. Biol. 159:601 621 and glutamine synthetase (GS)-cells used with a GS selection marker, e.g., described in Lin, et al., MAbs. (2019) 11(5):965-976; and Noh, et al., Sci Rep. (2018) 8(1):5361), human embryonic kidney 293 cells (e.g., 293, 293E, 293T, Expi293™), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell. CHO and NS0 cell lines for recombinant antibody production are reviewed by Dhara, et al., BioDrugs. (2018) 32(6):571-584.

In an exemplary system for expression of the proteins of interest, recombinant expression vectors encoding the first and second polypeptide chains (e.g., VH and VL of a first antigen binding domain and VH and VL of a second antigen binding domain) are introduced into dhfr-CHO cells by calcium phosphate-mediated transfection. In a specific embodiment, the dhfr-CHO cells are cells of the DG44 cell line, such as DG44i (see, e.g., Derouaz et al., Biochem Biophys Res Commun., (2006) 340(4):1069-77). Within the recombinant expression vectors, the first and second polypeptide chains are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vectors also carry a DHFR gene, which allows for selection of CHO cells that have been transfected with the vector using methotrexate selection/amplification. The selected transformant host cells are cultured to allow for expression of the proteins of interest, which can be recovered from the culture medium. In one embodiment, three polypeptide chains of a multi-specific antigen binding molecule, as described herein, are expressed in a single cell. In one embodiment, three polypeptide chains of a multi-specific antigen binding molecule, as described herein, are expressed in a single cell from a single vector.

The biologic molecules of interest can be isolated from inside or outside (such as medium) of the host cell and purified as substantially pure and homogenous, non-aggregated biologic molecules of interest (e.g., antibodies, Fc fusion proteins, multi-specific binding molecules). As appropriate or desired, the cell or population of cells are cultured in a culture volume of at least 2 L, e.g., at least 5 L, 10 L, 50 L, 100 L, 150 L, 200 L, 250 L, or more.

Using the modified IRES sequences, as described herein, in some embodiments, at least 1 g/L e.g., at least 1.2 g/L, at least 1.4 g/L, at least 1.6 g/L, at least 1.8 g/L, at least 2.0 g/L, at least 3.0 g/L, at least 4.0 g/L, at least 5.0 g/L, at least 6.0 g/L, at least 7.0 g/L, at least 8.0 g/L, at least 9.0 g/L, of the protein of interest is expressed from the host cell. In some embodiments, at least 2-fold, e.g., at least 5-fold, at least 10-fold, at least 15-fold, at least 20-fold, of the protein of interest is expressed from the host cell comprising an expression cassette comprising a modified IRES, in comparison to a host cell comprising an expression cassette comprising a wild-type encephalomyocarditis virus (EMCV) IRES.

In some embodiments, expression levels of the biologic molecules can be further increased by using in combination with gene-amplification (Noh, et al., Sci Rep (2018) 8:5361) or transposon-mediated expression (Balasubramanian, et al., Biotechnol Bioeng (2016) 113:1234-1243).

### 7. Kits

Further provided are kits comprising one or more vectors or host cells comprising one or more expression cassettes comprising one or more of the modified IRES sequences, as described herein.

In one embodiment, the kit comprises one or more containers (e.g., vials, ampules) containing one or more of the vectors or host cells provided herein. Optionally associated with such container(s) can be a description and instructions for use.

### EXAMPLES

The following examples are offered to illustrate, but not to limit the claimed invention.

### Example 1

### Modified IRES Elements Elevated GS Selection Stringency and Enabled Preferential Enrichment of High-Expression Cell Population For GFP

In this example, we evaluated how IRES modification affected stable pool selection stringency and the impact on the expression levels of green fluorescent protein (GFP).

### Methods

To do this, we constructed plasmid vectors containing an expression cassette with the following operably linked elements in the 5′ to 3′ direction: CMV promoter, GFP coding sequence (CDS), internal ribosome entry site (IRES) (wild type or modified variant), hamster glutamine synthetase (GS) CDS, and SV40 polyadenylation signal (CMVp-GFP CDS-IRES (wild type or modified variant)-hamster GS CDS-SV40 polyA). The modified IRES element physically linked the CDS of GFP and GS together and allowed the two genes to be transcribed together as a single mRNA transcript. This genetic arrangement ensured that GFP and GS were always co-expressed within a cell, and eliminated the possibility of transfected cells only expressing the GS marker without any GFP expression.

Endotoxin-free DNA was prepared for GFP-expression vectors containing different IRES variants, and transfected into GS knock-out Chinese hamster ovary (CHO)-K1 host cells using the NEON™ electroporation apparatus (Invitrogen) according to the manufacturer's recommended protocol. After 24 hours recovery in non-selective growth medium, the post-electroporation cells were centrifuged and resuspended in glutamine-free selective medium, and continuously sub-cultured every 3 or 4 days, to select for bulk pools in which the expression vector has been stably integrated into the host cell genome. After the bulk pool culture viability reached above 90%, research cell banks (RCB) were cryo-preserved and subsequently revived for analysis and characterization.

In all examples, the tested variant IRES polynucleotide sequences are provided in Table A.

### Results

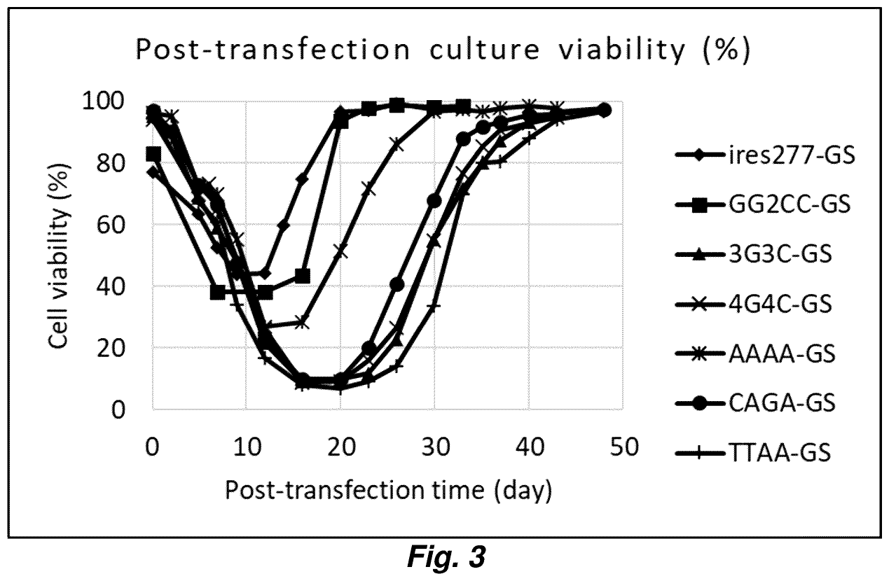

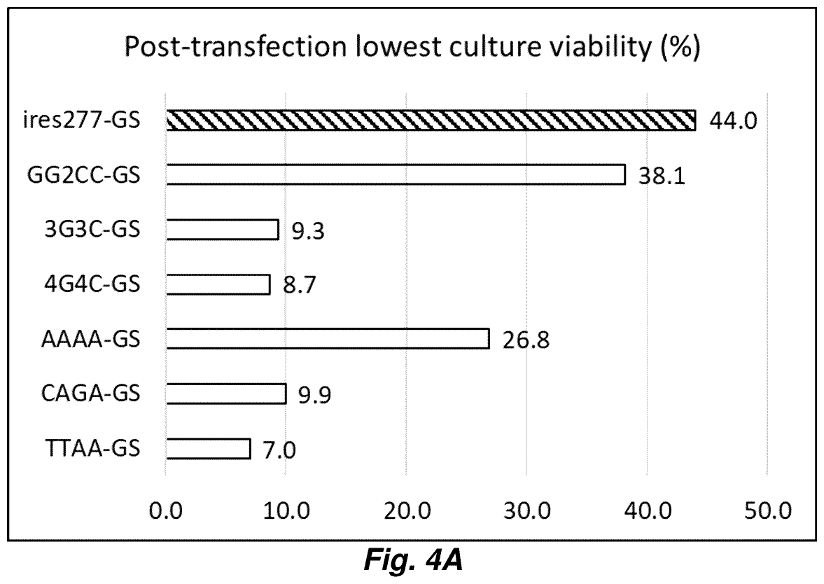

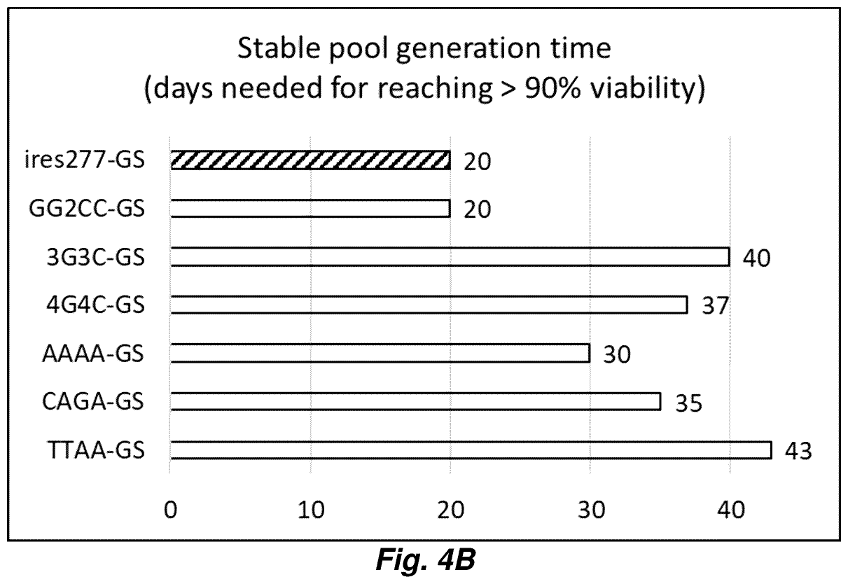

As shown in FIG. 3, cells transfected with expression vectors containing different IRES variants displayed very different cell viability and growth profiles during the post-transfection selection period. Stable pools transfected with the wild-type IRES vector displayed a brief phase of viability reduction and reached its minimal culture viability of 44% on day 9, and then quickly resumed active growth and reached over 90% culture viability on day 20 (FIG. 4A). In contrast, most IRES-variant transfected pools exhibited a more extended viability reduction phase, displayed substantially lower minimal culture viability levels (<10%), and took much more time (up to 43 days) to reach 90% culture viability (FIG. 4B). These results demonstrated that connecting GFP and GS via these modified IRES elements enabled substantially elevated GS selection stringency post transfection, resulting in much more effective elimination of the subset cell populations expressing low levels of GS protein.

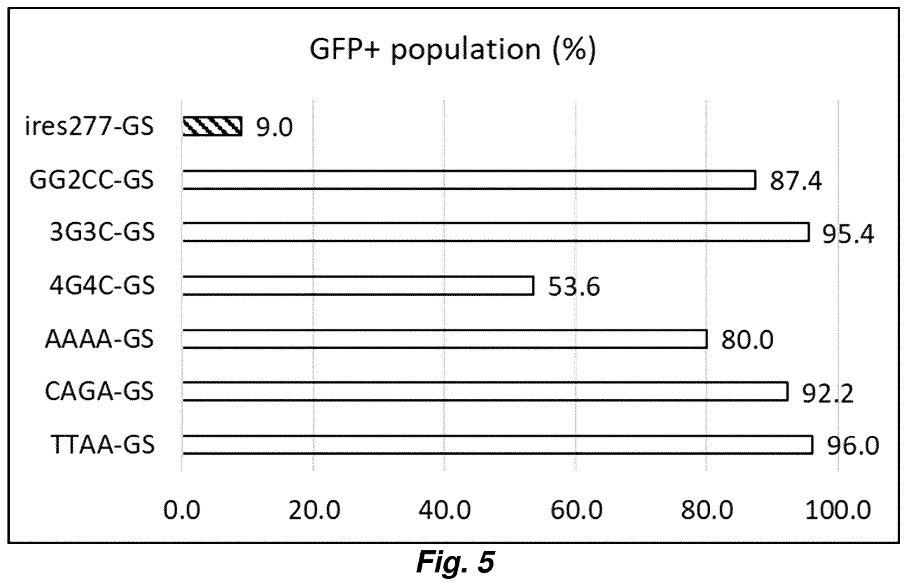

To determine if these modified IRES could also enrich high GFP expressing cell population, we thawed the cryo-preserved RCBs for all transfected bulk pools, sub-cultured them for at least two passages to ensure each revived bulk pool resume active growth, and then took cell samples and subjected them to fluorescence-activated cell sorting (FACS) analysis to analyze GFP expression levels on a single-cell basis. As summarized in FIG. 5, only 9% of the cells transfected by the wild-type IRES expression vector display positive GFP signal, whereas much higher percentage (ranging from 53.6 to 96.0%) of cells transfected with different IRES-variant containing vector were GFP-positive. These results demonstrated that these modified IRES elements, when used as described here, are capable of generating transfected stable pools with high levels of GFP expression. These data also indicated their potential utility in improving the productivity of recombinant proteins that are more relevant to the biopharmaceutical industry, including antibodies, multi-specific immunoglobulins, immunoadhesins and fusion proteins.

### Example 2

### Modified IRES Elements Enabled Preferential Enrichment of High-Producing Cell Population for an Fc Fusion Protein

In this example, we evaluated how IRES modification affected the expression level of an Fc fusion protein (hereinafter “Fc-1”).

### Methods

To do this, we constructed plasmid vectors containing an expression cassette with the following operably linked elements in the 5′ to 3′ direction: CMVp-Fc-1 CDS-IRES (wild type or modified variant)-hamster GS CDS-SV40 polyadenylation signal (polyA). The modified IRES element physically linked the coding sequences of Fc-1 and GS together and allowed the two genes to be transcribed together as a single mRNA transcript. This genetic arrangement ensured that Fc-1 and GS were always co-expressed within a cell, and eliminated the possibility of transfected cells only expressing the GS marker without any Fc-1 expression.

Endotoxin-free DNA was prepared for Fc-1 expression vectors containing different IRES variants, and transfected into GS knock-out Chinese hamster ovary (CHO)-K1 host cells using the NEON™ electroporation apparatus (Invitrogen) according to the manufacturer's recommended protocol. After 24 hours recovery in non-selective growth medium, the post-electroporation cells were centrifuged and resuspended in glutamine-free selective medium, and continuously sub-cultured every 3 or 4 days, to select for bulk pools in which the expression vector has been stably integrated into the host cell genome. After the bulk pool culture viability reached above 90%, RCBs were cryo-preserved and subsequently revived for analysis and characterization.

To determine the productivity of Fc-1, we thawed the cryo-preserved RCBs for all transfected bulk pools, sub-cultured them for at least two passages to ensure each revived bulk pool resume active growth, and then subjected them to a standard 14-day fed-batch production process, using Ex-CELL Adv. CHO Fed-Batch Medium (SAFC) as basal medium, and Cell Boost Feed 7a & 7b (Cytiva) as feed media. On day 0 of the fed-batch process, 24-well deep-well plates were inoculated by different bulk pool culture at a seeding density of 1×10⁶ cells per milliliter. The cell cultures were initially incubated at 37° C., and then switched to 34° C. from day 3 to day 14. Cell Boost Feed 7a & 7b were fed to each culture on day 2, day 5, day 7 and day 9 according to the manufacturer's recommendation, and cell counts, culture metabolites (including glucose, lactate, ammonia levels) were monitored on days 2, 5, 7, 9, and 12. Extra glucose was supplemented to the cultures on day 0 (to 12 g/L), day 9 (to 18 g/L) and day 12 (to 14 g/L), to minimize the potential risk of glucose depletion during the process. The fed-batch cultures were harvested on day 14 (or on the day when the culture viability dropped to below 50%), and the harvested cell culture fluid (HCCF) were used to determine IgG expression levels using the Cedex Bio HT® analyzer.

### Results

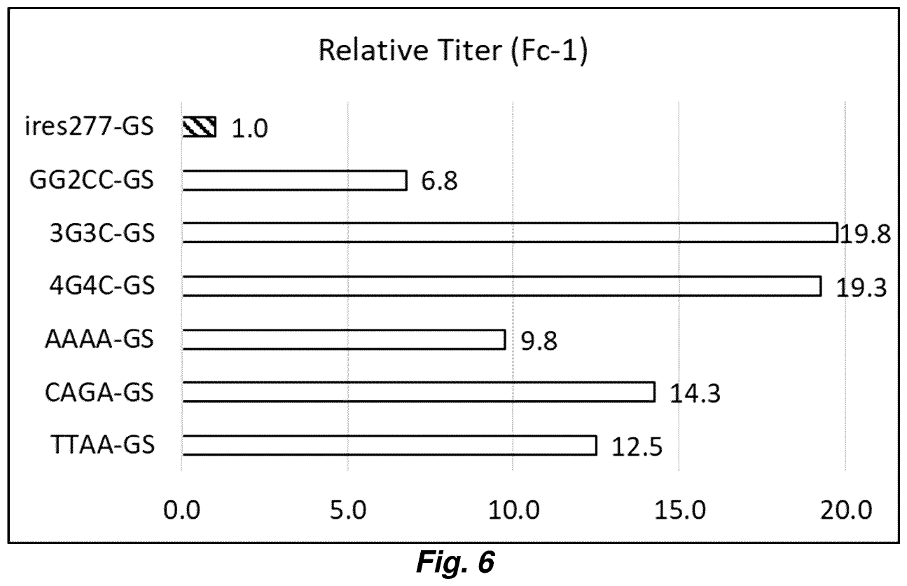

The relative Fc-1 expression levels, normalized against the Fc-1 expression level observed from the wild-type IRES containing vector, are shown in FIG. 6. Similar to the GFP-expression results depicted in FIG. 5 and described in Example 1, bulk pools transfected by expression vectors containing different IRES variants displayed significantly higher Fc-1 expression levels (ranging from 6.8-fold to 19.8-fold higher) in comparison with the expression level displayed by the stable pool generated from the wild-type IRES containing vector. These results confirmed that the modified IRES variants described herein are indeed capable of generating stably transfected cell pools expressing high levels of the Fc fusion protein Fc-1. This class of Fc fusion proteins, namely immunoadhesins, is highly relevant to the biopharmaceutical industry.

### Example 3

### Modified IRES Elements Enabled Preferential Enrichment of High-Producing Cell Population for Monoclonal Antibody (mAb-1)

In this example, we evaluated how IRES modification affected the expression level of a therapeutic monoclonal antibody (mAb-1).

### Methods

To do this, we constructed plasmid vectors containing dual-expression cassette with the following operably linked elements in the 5′ to 3′ direction: CMVp-mAb-1 light chain (LC) CDS-BGH polyadenylation signal (polyA)-CMVp-mAb-1 heavy chain (HC) CDS-IRES (wild type or modified variant)-GS CDS-SV40 polyA. The modified IRES element physically linked the CDS of mAb-1 HC and GS together and allowed the two genes to be transcribed together as a single mRNA transcript. This genetic arrangement ensured that HC and GS were always co-expressed within a cell, and eliminates the possibility of cells only expressing the GS marker without any HC expression. A traditional non-IRES expression vector, which has the following operably linked elements in the 5′ to 3′ direction: CMVp-mAb-1 light chain (LC) CDS-BGH polyA-CMVp-mAb-1 heavy chain (HC) CDS-BGH polyA-SV40 promoter (SV40p)-GS CDS-SV40 polyA, was used as a comparator.

Endotoxin-free DNA was prepared for mAb-1 expression vectors containing different IRES variants, as well as the control non-IRES vector, and transfected into GS knock-out Chinese hamster ovary (CHO)-K1 host cells using the NEON™ electroporation apparatus (Invitrogen) according to manufacture recommended protocol. After 24 hours recovery in non-selective growth medium, the post-electroporation cells were centrifuged and resuspended in glutamine-free selective medium, and continuously sub-cultured every 3 or 4 days, to select for bulk pools in which the expression vector has been stably integrated into the host cell genome. After the bulk pool culture viability reached above 90%, RCBs were cryo-preserved and subsequently revived for analysis and characterization.

To determine the productivity of mAb-1, we thawed the cryo-preserved RCBs for all transfected bulk pools, sub-cultured them for at least two passages to ensure each revived bulk pool resume active growth, and then subjected them to a standard 14-day fed-batch production process, using Ex-CELL Adv. CHO Fed-Batch Medium (SAFC) as basal medium, and Cell Boost Feed 7a & 7b (Cytiva) as feed media. On day 0 of the fed-batch process, 24-well deep-well plates were inoculated by different bulk pool culture at a seeding density of 1×10⁶ cells per milliliter. The cell cultures were initially incubated at 37° C., and then switched to 34° C. from day 3 to day 14. Cell Boost Feed 7a & 7b were fed to each culture on day 2, day 5, day 7 and day 9 according to manufacturer's recommendation, and cell counts, culture metabolites (including glucose, lactate, ammonia levels) were monitored on days 2, 5, 7, 9, and 12. Extra glucose was supplemented to the cultures on day 0 (to 12 g/L), day 9 (to 18 g/L) and day 12 (to 14 g/L), to minimize the potential risk of glucose depletion during the process. The fed-batch cultures were harvested on day 14 (or on the day when the culture viability dropped to below 50%), and the harvested cell culture fluid (HCCF) were used to determine IgG expression level using the Cedex Bio HT® analyzer.

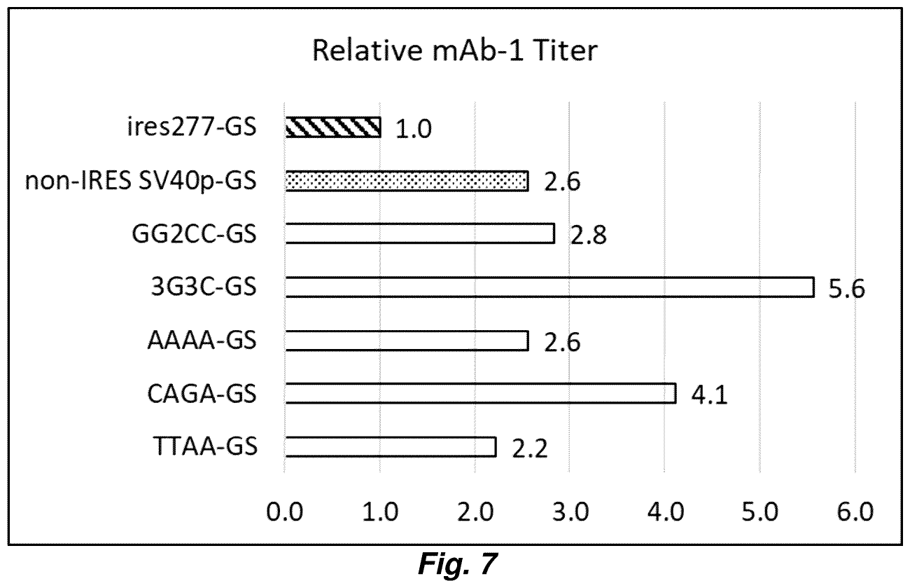

The relative mAb-1 expression levels, normalized against the mAb-1 expression level observed from the non-IRES control vector, are shown in FIG. 7. Bulk pools transfected by expression vectors containing different IRES variants displayed significantly higher mAb-1 expression levels (ranging from 2.2-fold to 5.6-fold) than the expression level displayed by the stable pool generated from the wild-type IRES containing vector. Compared to the traditional non-IRES vector with GS gene independently transcribed from the SV40 promoter, the stable pools generated from vectors containing the CAGA and 3G3C variants displayed, respectively, 1.6-times and 2.2-times of mAb-1. These results confirmed that the modified IRES variants described herein are indeed capable of generating stably transfected cell pools expressing high levels of mAb-1. This class of therapeutic proteins, namely monoclonal antibodies, is highly relevant to the biopharmaceutical industry.

### Example 4

### Using Non-Hamster GS Genes as Selection Marker to Further Enrich High-Producing Cell Population

In this example, we assessed if GS genes from non-hamster organisms could be used as alternative GS selectable markers to further enrich a high-producing cell population and increase product expression level in our IRES-based expression system.

The rationale behind this experiment is as follows. With the amino acid sequence divergence from the hamster GS, non-hamster GS proteins will very likely exhibit reduced levels of the GS enzymatic activity in CHO cells. To compensate for the reduced enzymatic activity, CHO cells need to express higher level of the non-hamster GS protein to support growth in glutamine-free mediaum (a standard selective condition). Furthermore, combining non-hamster GS with the herein described IRES variants will have at least an additive effect in further increasing the expression level of therapeutic proteins in CHO cells.

### Methods

To do this, we constructed plasmid vectors containing dual-expression cassette with the following operably linked elements in the 5′ to 3′ direction: CMVp-mAb-1 light chain (LC) CDS-BGH polyA signal-CMVp-mAb-1 heavy chain (HC) CDS-IRES (wild-type or modified variant)-GS (hamster or non-hamster) CDS-SV40 polyA signal. The modified IRES element physically linked the CDS of mAb-1 HC and GS together, allowing the two genes to be transcribed together as a single mRNA transcript. This genetic arrangement ensured that HC and GS were always co-expressed within a cell, and eliminated the possibility of cells only expressing the GS marker without any HC expression.

Endotoxin-free DNA was prepared for mAb-1 expression vectors containing different IRES variants and hamster or non-hamster GS gene, then transfected into GS knock-out Chinese hamster ovary (CHO)-K1 host cells using the NEON™ electroporation apparatus (Invitrogen) according to the manufacturer's recommended protocol. After 24 hours recovery in non-selective growth medium, the post-electroporation cells were centrifuged and resuspended in glutamine-free selective medium, and continuously sub-cultured every 3 or 4 days, to select for bulk pools in which the expression vector has been stably integrated into the host cell genome. After the bulk pool culture viability reached above 90%, RCBs were cryo-preserved and subsequently revived for analysis and characterization.

To determine the productivity of mAb-1, we thawed the cryo-preserved RCBs for all transfected bulk pools, sub-cultured them for at least two passages to ensure each revived bulk pool resume active growth, and then subjected them to a standard 14-day fed-batch production process, using Ex-CELL Adv. CHO Fed-Batch Medium (SAFC) as basal medium, and Cell Boost Feed 7a & 7b (Cytiva) as feed media. On day 0 of the fed-batch process, 24-well deep-well plates were inoculated by different bulk pool culture at a seeding density of 1×10⁶ cells per milliliter. The cell cultures were initially incubated at 37° C., and then switched to 34° C. from day 3 to day 14. Cell Boost Feed 7a & 7b were fed to each culture on day 2, day 5, day 7 and day 9 according to manufacturer's recommendation, and cell counts, culture metabolites (including glucose, lactate, ammonia levels) were monitored on days 2, 5, 7, 9, and 12. Extra glucose was supplemented to the cultures on day 0 (to 12 g/L), day 9 (to 18 g/L) and day 12 (to 14 g/L), to minimize the potential risk of glucose depletion during the process. The fed-batch cultures were harvested on day 14 (or on the day when the culture viability dropped to below 50%), and the harvested cell culture fluid (HCCF) were used to determine IgG expression level using the Cedex Bio HT® analyzer.

### Results

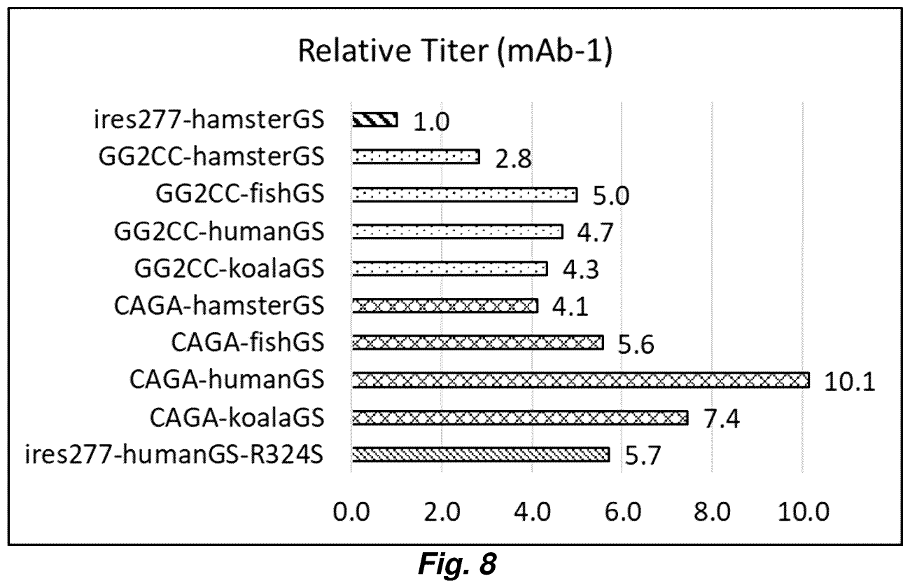

The relative mAb-1 expression levels, normalized against the mAb-1 expression level observed from the wild type-IRES (ires277) hamster GS control vector, are shown in FIG. 8. As demonstrated in previous examples, bulk pools transfected by expression vectors containing IRES variants (GG2CC & CAGA) and hamster GS gene displayed higher mAb-1 expression levels (2.8-fold and 4.1-fold, respectively) than the expression level displayed by the stable pool generated from the wild-type IRES and hamster GS containing vector. More interestingly, using GS genes from non-hamster organisms (fish, human, and koala) substantially increased the bulk pool expression levels further for both GG2CC and CAGA IRES variants (4.3 to 5.0-fold, and 5.6 to 10.1-fold, respectively). Furthermore, even with the wild type IRES, using the mutant human GS gene (human GS R324S) resulted in a dramatic expression level increase (5.7-fold) as well. These results confirmed and validated the hypothesis that non-hamster GS genes can be used as GS selection markers, and that they have at least an additive effect with the modified IRES variants described herein in further improving cell line productivities for mAb-1. This class of therapeutic proteins, namely monoclonal antibodies, is highly relevant to the biopharmaceutical industry.

### Example 5

### Identifying IRES-Variants to Further Improve Cell Population Expression Levels

The GG2CC variant was originally introduced to IRES by Jang and Wimmer (1990) in their structure-function study of the wild type IRES element. Specifically, the “GG” to “CC” mutations were made to disrupt the structure of stem-loop E, which is essential for IRES function (Jang and Wimmer, 1990, GENES & DEVELOPMENT 4:1560-1572). Similarly, the CAGA, AAAA, and TTAA variants were originally described by Robertson et al. (1999) as part of their characterization of the IRES GNRA tetraloop, which plays a critical role in IRES function (Robertson et al. 1999, RNA 5:1167-1179).

Having demonstrated these IRES variants worked well in improving cell line productivities in the above examples, we evaluated additional IRES variants, as described herein within or near stem-loop E and GNRA tetraloop regions, to identify improved IRES variants for enriching high-producing cell populations in our system.

### Methods

To do this, we used an RNA secondary structure prediction software (RNAfold), and designed a number of IRES variants with predicted changes in the secondary structure of stem-loop E or GNRA tetraloop. Subsequently, we constructed plasmid vectors containing dual-expression cassette with the following operably linked elements in the 5′ to 3′ direction: CMVp-mAb-2 light chain (LC) CDS-IRES (modified variant)-human GS-R324S CDS-BGH polyA signal-CMVp-mAb-2 heavy chain (HC) CDS-SV40 polyA signal. The modified IRES element physically linked the CDS of mAb-2 LC and GS together, allowing the two genes to be transcribed together as a single mRNA transcript. This genetic arrangement ensured that LC and GS were always co-expressed within a cell, and eliminated the possibility of cells only expressing the GS marker without any LC expression. A traditional non-IRES expression vector, which has the following operably linked elements in the 5′ to 3′ direction: CMVp-mAb-2 light chain (LC) CDS-BGH polyA signal-CMVp-mAb-2 heavy chain (HC) CDS-BGH polyA signal-SV40p-hamster GS CDS-SV40 polyA signal, was used as a comparator.

Endotoxin-free DNA was prepared for mAb-2 expression vectors containing different IRES variants and hamster or non-hamster GS gene, then transfected into GS knock-out Chinese hamster ovary (CHO)-K1 host cells using the NEON™ electroporation apparatus (Invitrogen) according to the manufacturer's recommended protocol. After 24 hours recovery in non-selective growth medium, the post-electroporation cells were centrifuged and resuspended in glutamine-free selective medium, and continuously sub-cultured every 3 or 4 days, to select for bulk pools in which the expression vector has been stably integrated into the host cell genome. After the bulk pool culture viability reached above 90%, RCBs were cryo-preserved and subsequently revived for analysis and characterization.

To determine the productivity of mAb-2, we thawed the cryo-preserved RCBs for all transfected bulk pools, sub-cultured them for at least two passages to ensure each revived bulk pool resume active growth, and then subjected them to a standard 14-day fed-batch production process, using Ex-CELL Adv. CHO Fed-Batch Medium (SAFC) as basal medium, and Cell Boost Feed 7a & 7b (Cytiva) as feed media. On day 0 of the fed-batch process, 24-well deep-well plates were inoculated by different bulk pool culture at a seeding density of 1×10⁶ cells per milliliter. The cell cultures were initially incubated at 37° C., and then switched to 34° C. from day 3 to day 14. Cell Boost Feed 7a & 7b were fed to each culture on day 2, day 5, day 7 and day 9 according to manufacturer's recommendation, and cell counts, culture metabolites (including glucose, lactate, ammonia levels) were monitored on days 2, 5, 7, 9, and 12. Extra glucose was supplemented to the cultures on day 0 (to 12 g/L), day 9 (to 18 g/L) and day 12 (to 14 g/L), to minimize the potential risk of glucose depletion during the process. The fed-batch cultures were harvested on day 14 (or on the day when the culture viability dropped to below 50%), and the harvested cell culture fluid (HCCF) were used to determine IgG expression level using the Cedex Bio HT® analyzer.

### Results

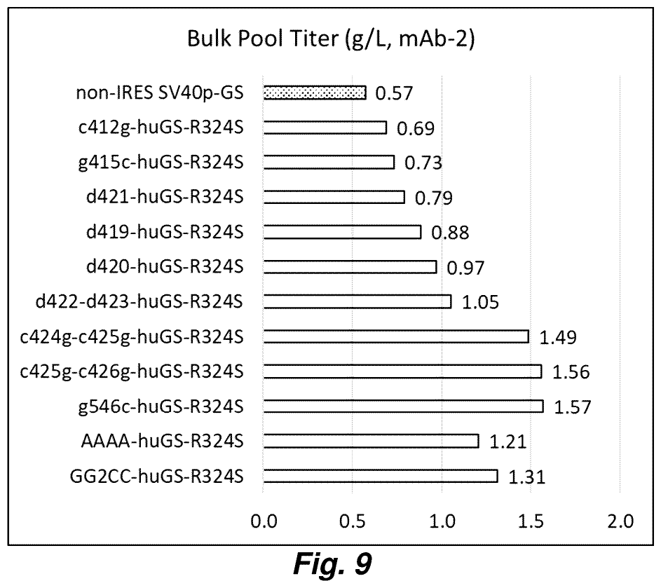

FIG. 9 summarizes the fed-batch mAb-2 expression levels observed from bulk pools transfected with vectors containing different IRES variants and GS genes. The control bulk pool, which was generated from the traditional non-IRES vector with hamster GS gene independently transcribed from the SV40 promoter, displayed an expression level of 0.57 g/L, e.g., 0.6 g/L. In comparison, bulk pools derived from expression vectors containing IRES variants (GG2CC, AAAA, and the newly designed variants) and human GS-R324S gene displayed a wide range of mAb-2 expression levels. Some newly designed IRES variants (c412g, g415c, and d421) produced expression levels comparable to the non-IRES control vector. On the other hand, vectors containing the following IRES variants, d422-d423, c424g-c425g, c425g-c426g, and g546c, produced much higher expression levels (from 1.05 to 1.57 g/L, e.g., 1.1 g/L to 1.6 g/L, or 1.8-fold to 2.8-fold of the non-IRES pool expression level). As expected, vectors containing the AAAA and GG2CC variants also gave rise to higher mAb-2 expression levels (1.21 and 1.31 g/L, e.g., 1.2 and 1.3 g/L, respectively). These results demonstrated that some of the newly designed herein described IRES variants, including d422-d423, c424g-c425g, c425g-c426g, and g546c, are capable of enriching high-producing cell populations in transfected cell pools, and significantly increasing the pool expression levels of mAb-2. This class of therapeutic proteins, namely monoclonal antibodies, is highly relevant to the biopharmaceutical industry.

### Example 6

### Identifying IRES-Variants for the Improved Expression of Fc-1

To further demonstrate the utility of the herein described IRES variants described in previous example, we examined their effects on the expression of a Fc fusion protein Fc-1.

### Methods

To do this, we constructed plasmid vectors containing an expression cassette with the following operably linked elements in the 5′ to 3′ direction: CMVp-Fc-1 CDS-IRES (modified variant)-human GS-R324S CDS-SV40 polyA signal. The modified IRES element physically linked the CDS of Fc-1 and GS together, and allowed the two genes to be transcribed together as a single mRNA transcript. This genetic arrangement ensured Fc-1 and GS were always co-expressed within a cell, and eliminated the possibility of cells only expressing the GS marker without any Fc-1 expression. A traditional non-IRES expression vector, which has the following operably linked elements: CMVp-Fc-1 CDS-BGH polyA signal-SV40p-hamster GS CDS-SV40 polyA signal, was used as a comparator.

Endotoxin-free DNA was prepared for Fc-1 expression vectors, and then transfected into GS knock-out Chinese hamster ovary (CHO)-K1 host cells using the NEON™ electroporation apparatus (Invitrogen) according to the manufacturer's recommended protocol. After 24 hours recovery in non-selective growth medium, the post-electroporation cells were centrifuged and resuspended in glutamine-free selective medium, and continuously sub-cultured every 3 or 4 days, to select for bulk pools in which the expression vector has been stably integrated into the host cell genome. After the bulk pool culture viability reached above 90%, RCB were cryo-preserved and subsequently revived for analysis and characterization.

To determine the productivity of Fe-1, we thawed the cryo-preserved RCBs for all transfected bulk pools, sub-cultured them for at least two passages to ensure each revived bulk pool resume active growth, and then subjected them to a standard 14-day fed-batch production process, using Ex-CELL Adv. CHO Fed-Batch Medium (SAFC) as basal medium, and Cell Boost Feed 7a & 7b (Cytiva) as feed media. On day 0 of the fed-batch process, 24-well deep-well plates were inoculated by different bulk pool culture at a seeding density of 1×10⁶ cells per milliliter. The cell cultures were initially incubated at 37° C., and then switched to 34° C. from day 3 to day 14. Cell Boost Feed 7a & 7b were fed to each culture on day 2, day 5, day 7 and day 9 according to manufacturer's recommendation, and cell counts, culture metabolites (including glucose, lactate, ammonia levels) were monitored on days 2, 5, 7, 9, and 12. Extra glucose were supplemented to the cultures on day 0 (to 12 g/L), day 9 (to 18 g/L) and day 12 (to 14 g/L), to minimize the potential risk of glucose depletion during the process. The fed-batch cultures were harvested on day 14 (or on the day when the culture viability dropped to below 50%), and the harvested cell culture fluid (HCCF) were used to determine IgG expression level using the Cedex Bio HT® analyzer.

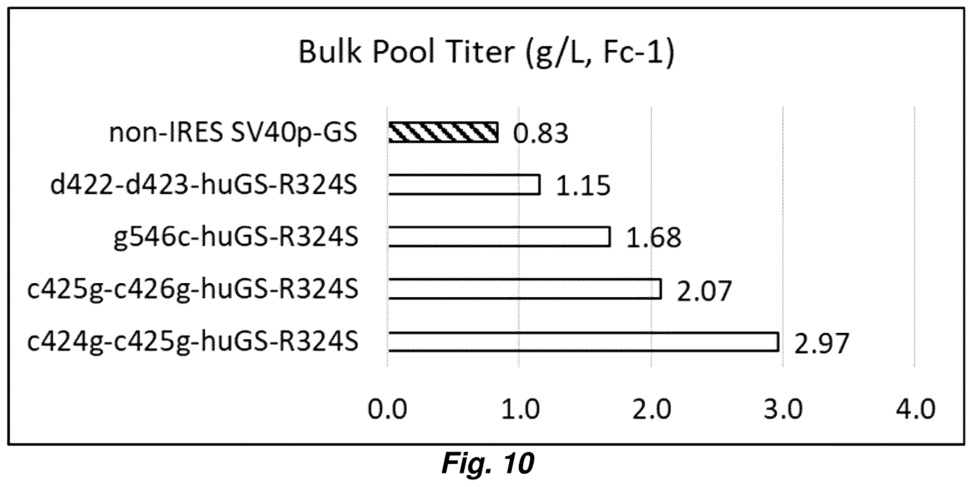

FIG. 10 summarizes the fed-batch Fc-1 expression levels observed from bulk pools transfected with vectors containing different IRES variants and GS genes. The control bulk pool, which was generated from the traditional non-IRES vector with hamster GS gene independently transcribed from the SV40 promoter, displayed an expression level of 0.83 g/L. In comparison, bulk pools derived from expression vectors containing the herein described IRES variants (d422-d423, c425g-c426g, g546c and c424g-c425g) and human GS-R324S gene displayed significantly higher expression levels, with the highest expression level reaching to 2.97 g/L, e.g., 3.0 g/L (3.6-fold of the non-IRES control expression level) from the c424g-c425g variant. These results demonstrated that these newly designed herein described IRES variants are capable of enriching high-producing cell populations in transfected cell pools, and significantly increasing the pool expression levels of Fc-1. This class of therapeutic proteins, namely Fc fusion proteins or immunoadhesins, is highly relevant to the biopharmaceutical industry.

### Example 7

### Identifying IRES-Variants for the Improved Expression of a Bispecific Fab-Targeted Cytokine

To further demonstrate the utility of the herein described IRES variants described in previous example, we examined their effects on the expression of a bispecific Fab-targeted cytokine, which is a heterodimeric molecule having a Fab-Fc polypeptide (LC1 and HC1) and a cytokine-Fc-fusion polypeptide (HC2), referred to as “bsAb-1”.

### Methods

To do this, we constructed plasmid vectors containing an expression cassette with the following operably linked elements in the 5′ to 3′ direction: CMVp-LC1 CDS-IRES (modified variant)-human GS-R324S CDS-BGH (bovine growth hormone) polyA signal. The modified IRES element physically linked the CDS of LC1 and GS together, and allowed the two genes to be transcribed together as a single mRNA transcript. This genetic arrangement ensured LC1 and GS were always co-expressed within a cell, and eliminated the possibility of cells only expressing the GS marker without any LC1 expression. The HC1 and HC2 genes were independently expressed from separate traditional non-IRES expression vectors, which have the following operably linked elements: CMVp-HC1 CDS-BGH polyA signal-SV40p-BSD (blasticidin resistence gene) CDS-SV40 polyA signal, and CMVp-HC2 CDS-BGH polyA signal-SV40p-BSD (blasticidin resistence gene) CDS-SV40 polyA signal, respectively.

Endotoxin-free DNA was prepared for LC1, HC1, and HC2 expression vectors, and then co-transfected into CHO-K1 GS-knockout cells using the NEON™ electroporation apparatus (Invitrogen) according to the manufacturer's recommended protocol. After 24 hours recovery in non-selective growth medium, the post-electroporation cells were centrifuged and resuspended in glutamine-free selective medium, and continuously sub-cultured every 3 or 4 days, to select for bulk pools in which the expression vector has been stably integrated into the host cell genome. After the bulk pool culture viability reached above 90%, RCB were cryo-preserved and subsequently revived for analysis and characterization.

To determine the productivity of bsAb-1, we thawed the cryo-preserved RCBs for all transfected bulk pools, sub-cultured them for at least two passages to ensure each revived bulk pool resume active growth, and then subjected them to a standard 14-day fed-batch production process, using Ex-CELL Adv. CHO Fed-Batch Medium (SAFC) as basal medium, and Cell Boost Feed 7a & 7b (Cytiva) as feed media. On day 0 of the fed-batch process, 24-well deep-well plates were inoculated by different bulk pool culture at a seeding density of 1×10⁶ cells per milliliter. The cell cultures were initially incubated at 37° C., and then switched to 34° C. from day 3 to day 14. Cell Boost Feed 7a & 7b were fed to each culture on day 2, day 5, day 7 and day 9 according to manufacturer's recommendation, and cell counts, culture metabolites (including glucose, lactate, ammonia levels) were monitored on days 2, 5, 7, 9, and 12. Extra glucose were supplemented to the cultures on day 0 (to 12 g/L), day 9 (to 18 g/L) and day 12 (to 14 g/L), to minimize the potential risk of glucose depletion during the process. The fed-batch cultures were harvested on day 14 (or on the day when the culture viability dropped to below 50%), and the harvested cell culture fluid (HCCF) were used to determine IgG expression level using the Cedex Bio HT® analyzer.

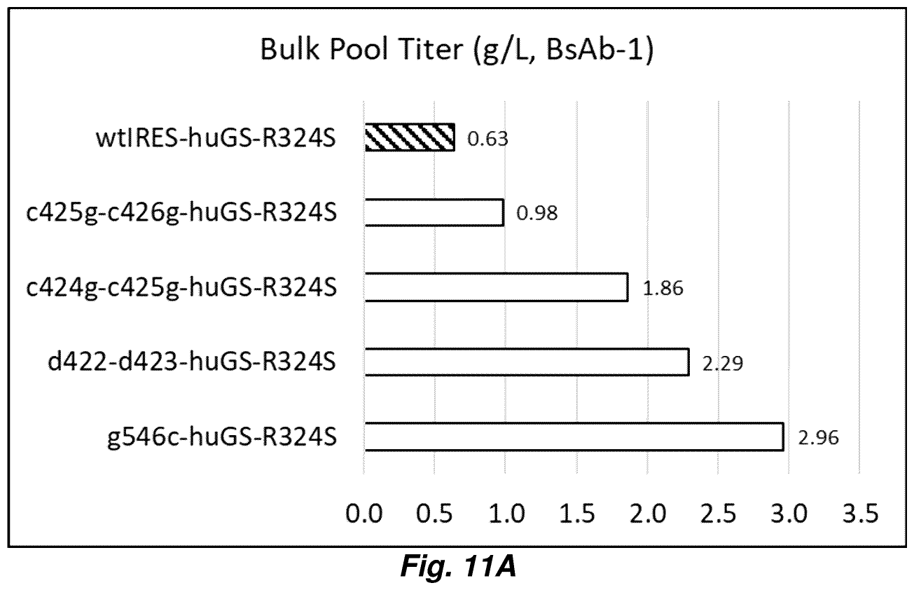

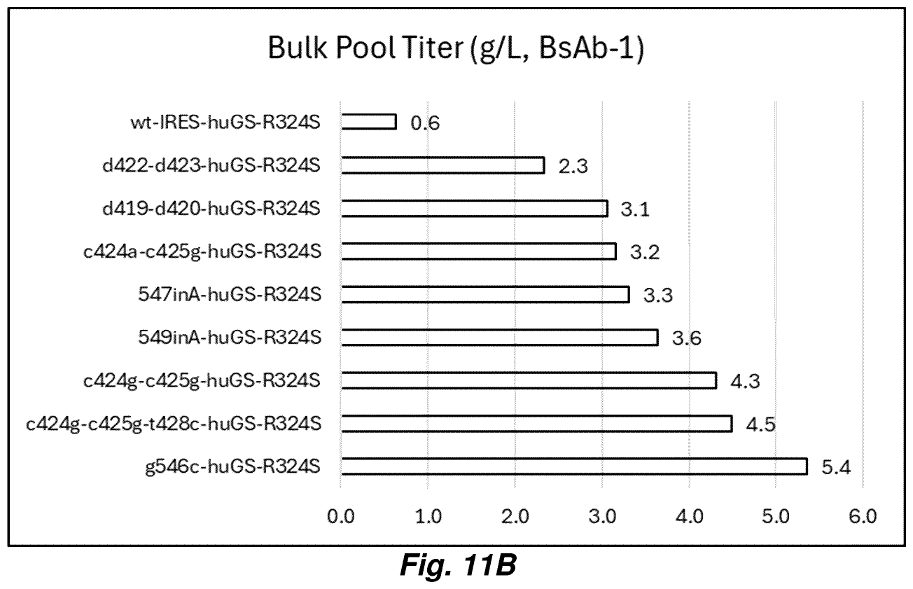

FIGS. 11A-11B depict the fed-batch bsAb-1 expression levels observed from bulk pools transfected with vectors containing different IRES variants and the human GS-R324S gene as the selectable marker. The control bulk pool, which was generated from the wild-type (un-modified) IRES vector, displayed an expression level of 0.63 g/L, e.g., 0.6 g/L. In comparison, bulk pools derived from expression vectors containing IRES variants (d422-d423, d419-d420, c424a-c425g, 547inA, 549inA, c424g-c425g, c424g-c425g-t428c, and g546c) displayed higher expression levels, with the highest expression level reaching to 2.96 g/L, e.g., 3.0 g/L (4.7-fold), as high as 5.4 g/L (9.0-fold), of the wild-type IRES control expression level) from the g546c variant. These results demonstrate that the herein described IRES variants enriched high-producing cell populations in transfected cell pools, and significantly increasing the pool expression levels of the bsAb-1 molecule. This class of therapeutic proteins, namely targeted cytokines, is highly relevant to the biopharmaceutical industry.

### Example 8

### Different Selectable Markers Require Different IRES-Variants for Improved Enhancement of Protein Expression

From experiments described in Examples 5 to 7, we also identified several IRES variants that, when coupled with the mutated human GS-R324S selectable marker, failed to generate viable stable pools and resulted in cell death in glutamine-free selective medium. These results suggested that these IRES variants, which included d547, d549, d422-d423-g546c, and 546inA, were unable to translate enough human GS-R324S enzyme to support cell growth in glutamine-free medium.

### Methods

To evaluate whether these IRES variants could be used in combination of other selectable markers, we constructed plasmid vectors containing the Fc-1 expression cassette with the following operably linked elements in the 5′ to 3′ direction: CMVp-Fc-1 CDS-IRES (modified variant)-ZeoR CDS-BGH (bovine growth hormone) polyA signal. The modified IRES element physically linked the CDS of Fc-1 and Zeocin-resistance gene (ZeoR) together, and allowed the two genes to be transcribed together as a single mRNA transcript. This genetic arrangement ensured Fc-1 and ZeoR were always co-expressed within a cell, and eliminated the possibility of cells only expressing the Zeocin marker without any Fc-1 expression.

We also constructed plasmid vectors containing the bsAb-1 expression cassette with the following operably linked elements in the 5′ to 3′ direction: CMVp-bsAb-1-LC1 CDS-IRES (modified variant)-human GS-wt CDS-BGH (bovine growth hormone) polyA signal, or CMVp-bsAb-1-LC1 CDS-IRES (modified variant)-hamster GS CDS-BGH (bovine growth hormone) polyA signal. The modified IRES element physically linked the CDS of bsAb-1-LC1 and the human GS wild-type (wt) gene, or the hamster GS gene together, and allowed the LC1 and the human or hamster GS genes to be transcribed together as a single mRNA transcript. This genetic arrangement ensured LC1 and GS were always co-expressed within a cell, and eliminated the possibility of cells only expressing the GS marker without any LC1 expression. The bsAb-1-HC1 and bsAb-1-HC2 genes were independently expressed from separate IRES expression vectors, which have the following operably linked elements: CMVp-HC1 CDS-IRES (d549)-ZeoR-BGH polyA signal, and CMVp-HC2 CDS-IRES (d549)-ZeoR-BGH polyA signal, respectively.

Furthermore, we constructed plasmid vectors containing the VHH1-VHH2-Fc-1, which is a bispecific antibody comprising a first single immunoglobulin variable domain (VHH1) and a second VHH (VHH2). The VHH1-VHH2-Fc-1 expression cassette has the following operably linked elements in the 5′ to 3′ direction: CMVp-VHH1-VHH2-Fc-1 CDS-IRES (modified variant)-human GS-wt CDS-BGH (bovine growth hormone) polyA signal, or CMVp-VHH1-VHH2-Fc-1 CDS-IRES (modified variant)-hamster GS CDS-BGH (bovine growth hormone) polyA signal. The modified IRES element physically linked the CDS of VHH1-VHH2-Fc-1 and the human GS wild-type (wt) gene, or the hamster GS gene together, and allowed the VHH1-VHH2-Fc-1 and the human or hamster GS genes to be transcribed together as a single mRNA transcript. This genetic arrangement ensured VHH1-VHH2-Fc-1 and GS were always co-expressed within a cell, and eliminated the possibility of cells only expressing the GS marker without any VHH1-VHH2-Fc-1 expression.

Endotoxin-free DNA was prepared for the Fc-1, bsAb-1-LC1, bsAb-1-HC1, bsAb-1-HC2, and VHH1-VHH2-Fc-1 expression vectors, and then transfected into CHO-K1 GS-knockout cells using the NEON™ electroporation apparatus (Invitrogen) according to the manufacturer's recommended protocol. After 24 hours recovery in non-selective growth medium, the post-electroporation cells were centrifuged and resuspended in either Zeocin-supplement selective medium (for Fc-1), or glutamine-free selective medium (for bsAb-1 and VHH1-VHH2-Fc-1), and continuously sub-cultured every 3 or 4 days, to select for bulk pools in which the expression vector has been stably integrated into the host cell genome. After the bulk pool culture viability reached above 90%, RCB were cryo-preserved and subsequently revived for analysis and characterization.

To determine the productivity of Fc-1, bsAb-1, or VHH1-VHH2-Fc-1, we thawed the cryo-preserved RCBs for all transfected bulk pools, sub-cultured them for at least two passages to ensure each revived bulk pool resume active growth, and then subjected them to a standard 14-day fed-batch production process, using Ex-CELL Adv. CHO Fed-Batch Medium (SAFC) as basal medium, and Cell Boost Feed 7a & 7b (Cytiva) as feed media. On day 0 of the fed-batch process, 24-well deep-well plates were inoculated by different bulk pool culture at a seeding density of 1×10⁶ cells per milliliter. The cell cultures were initially incubated at 37° C., and then switched to 34° C. from day 3 to day 14. Cell Boost Feed 7a & 7b were fed to each culture on day 2, day 5, day 7 and day 9 according to manufacturer's recommendation, and cell counts, culture metabolites (including glucose, lactate, ammonia levels) were monitored on days 2, 5, 7, 9, and 12. Extra glucose were supplemented to the cultures on day 0 (to 12 g/L), day 9 (to 18 g/L), and day 12 (to 14 g/L), to minimize the potential risk of glucose depletion during the process. The fed-batch cultures were harvested on day 14 (or on the day when the culture viability dropped to below 50%), and the harvested cell culture fluid (HCCF) were used to determine IgG expression level using the Cedex Bio HT® analyzer.

### Results

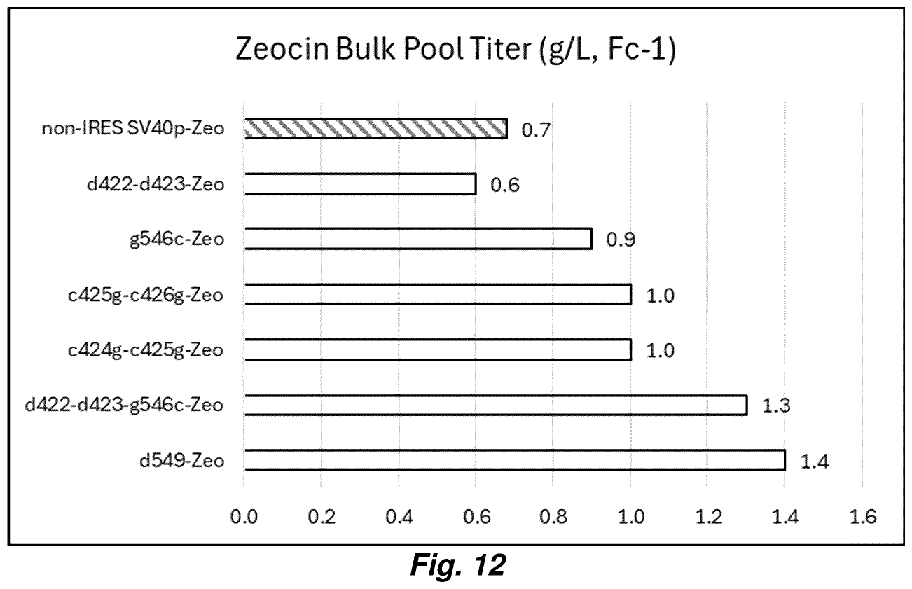

FIG. 12 depicts the fed-batch Fc-1 expression levels observed from bulk pools transfected with vectors containing different IRES variants and the ZeoR gene as the selectable marker. The control bulk pool, which was generated from the traditional non-IRES vector with ZeoR gene independently transcribed from the SV40 promoter, displayed an Fc-1 expression level of 0.7 g/L. Similar to results obtained from the human GS-R324S selectable marker (FIG. 10 in Example 6), Zeocin-resistant bulk pools derived from expression vectors containing IRES variants g546c, c425g-c426g, or c424g-c425g displayed Fc-1 titers ranging from 0.9 to 1.0 g/L (30-45% increase over the control). In addition, Zeocin-resistant bulk pools derived from expression vectors containing IRES variants d422-d-423-g546c and d549, both of which failed to generate stable pools with the human GS-R324S selectable marker, displayed higher Fe-1 titers ranging from 1.3 to 1.4 g/L (~100% increase over the control). These results demonstrate that the d422-d-423-g546c and d549 IRES variants enriched high-producing cell populations in Zeocin-resistant transfected cell pools, and increased the pool expression levels of the Fc-1 molecule.

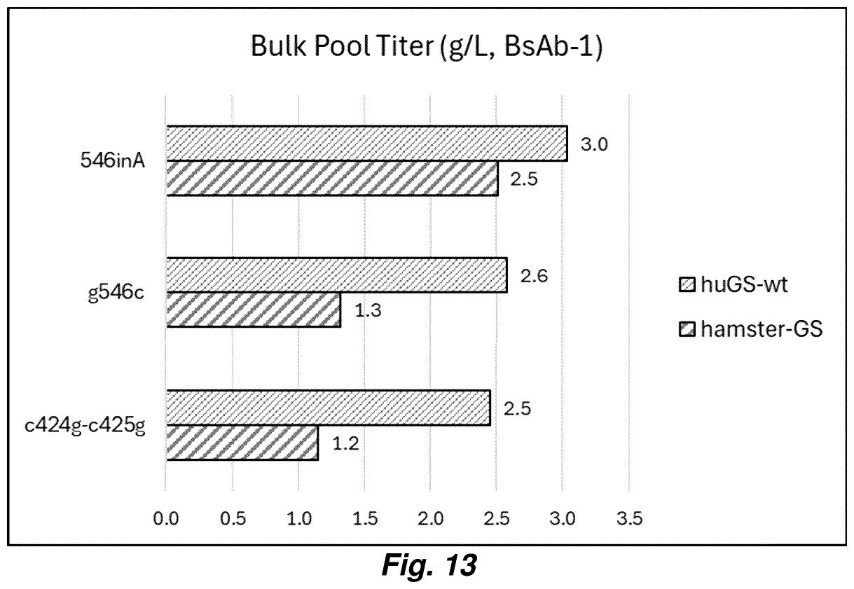

FIG. 13 depicts the fed-batch bsAb-1 expression levels observed from bulk pools transfected with vectors containing different IRES variants and the hamster or the wild-type human GS gene as the selectable marker. With the hamster GS selectable marker, bulk pools derived from expression vectors containing the 546inA IRES variant, which failed to generate stable pools when coupled with the mutated human GS-R324S gene, produced 2.2 g/L bsAb-1, approximately 100% higher than the titers displayed by bulk pools containing the c424g-c425g or the g546c variant. For the wild-type human GS selectable marker, bulk pools derived from expression vectors containing the 546inA IRES variant produced 3.0 g/L baAb-1, approximately 20% higher than the titers displayed by bulk pools containing the c424g-c425g or g546c variant.

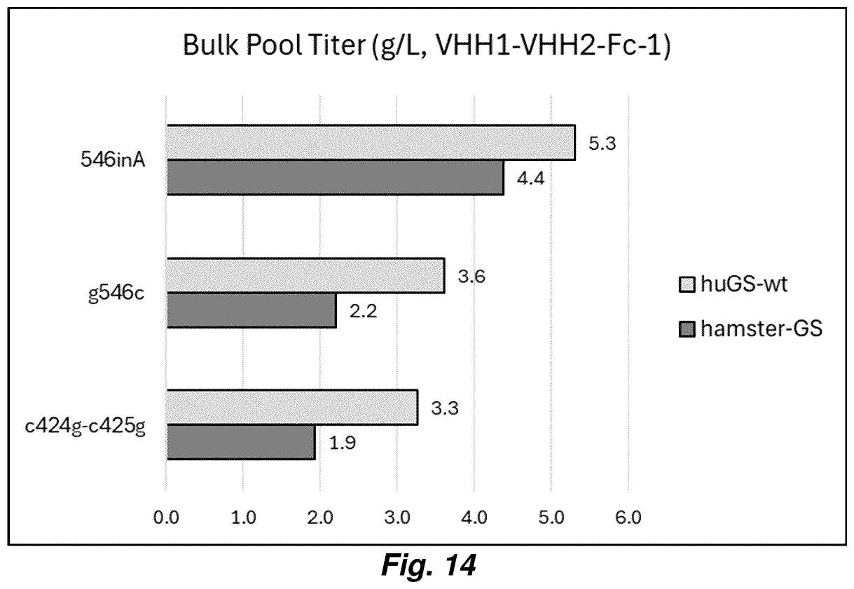

FIG. 14 depicts the fed-batch VHH1-VHH2-Fc-1 expression levels observed from bulk pools transfected with vectors containing different IRES variants and the hamster or the wild-type human GS gene as the selectable marker. With the hamster GS selectable marker, bulk pools derived from expression vectors containing the 546inA IRES variant, which failed to generate stable pools when coupled with the mutated human GS-R324S gene, produced 4.4 g/L VHH1-VHH2-Fc-1, approximately 100% higher than the titers displayed by bulk pools containing the c424g-c425g or the g546c variant. For the wild-type human GS selectable marker, bulk pools derived from expression vectors containing the 546inA IRES variant produced 5.3 g/L VHH1-VHH2-Fc-1, approximately 50% higher than the titers displayed by bulk pools containing the c424g-c425g or g546c variant.

Collectively, these results demonstrate that the 546inA IRES variant enriched high-producing cell populations in transfected cell pools with both hamster and wild-type human GS gene as the selectable marker, and increased the pool expression levels of the bsAb-1 and the VHH1-VHH2-Fc-1 molecules, both of which are highly relevant to the biopharmaceutical industry.

Results from FIGS. 12 to 14 also indicated that different selectable markers (ZeoR, hamster GS, wild type human GS, or mutated human GS-R324S) need to be combined with different IRES-variants to achieve maximal titer improvement.

### Example 9

### IRES-Variant Enables High Expression of a VHH1-VHH2-Fc-1 Fusion Molecule in Clonal Cell Lines

To further evaluate how IRES variant-containing vectors behave in clonal cell lines, we selected a representative vector containing an expression cassette with the following operably linked elements in the 5′ to 3′ direction: CMVp-VHH1-VHH2-Fc CDS-IRES (modified variant c424g-c425g)-human GS-R324S CDS-BGH (bovine growth hormone) polyA signal, a bispecific antibody comprising a first single immunoglobulin variable domain (VHH1) and a second VHH (VHH2).

### Methods

We transfected it into CHO-K1 GS-knockout host cells using the NEON™ electroporation apparatus (Invitrogen) according to the manufacturer's recommended protocol. After 24 hours recovery in non-selective growth medium, the post-electroporation cells were centrifuged and resuspended in glutamine-free selective medium, and continuously sub-cultured every 3 or 4 days, to select for bulk pools in which the expression vector has been stably integrated into the host cell genome. After the bulk pool culture viability reached above 90%, RCB were cryo-preserved and subsequently revived for analysis and characterization. After screening for titer and product quality, the top 8 pools were advanced into the single-cell cloning stage. Approximately 200 single-cell-originated clones were generated from each pool (for a total of ~1600 clones), and these clones were then subjected to productivity and product quality screening, including the 14-day fed-batch assay, to identify the top-performing single-cell-derived clones. HCCF of day 14 fed-batch cultures from each single-cell derived clone were used to determine overall titer, and then subjected to protein A affinity chromatography purification. The protein A purified materials were then subjected to size exclusion chromatography (SEC) and non-reduced capillary electrophoresis (nrCE) to determine high molecular weight (HMW) and low molecular weight (LMW) size-variant profiles of the VHH1-VHH2-Fc-1 fusion protein. The results are summarized in Table 1.

### Results

As shown in Table 1, the top 8 single-cell-derived clones displayed very favorable growth characteristics (11 to 23 1×10⁶ cells/ml and greater than 70% viability on day 14), very high productivity (5.86 to 9.06 g/L), and very low levels of HMW (lower than 1.72%) and LMW (lower than 1.83%) impurities. All these clones are suitable for clinical and commercial production of the VHH1-VHH2-Fc-1 molecule.

TABLE 1
Growth Characteristics and Product Quality Attributes of Clonal Cell Lines Comprising
Expression Cassette Comprising IRES Variant and Coding Sequence for VHH1-VHH2-Fc

| | FB Production | | | | | nrCE | | |
|---|---|---|---|---|---|---|---|---|
| | HPLC | FB D14 VCC | FB D14 | SEC | | | Main | |
| | Titer | (1 × 10⁶ | viability | % Main | % | LMW | Peak | HMW |
| Name | (g/L) | cells/mL) | (%) | Peak | HMW | % CPA | % CPA | % CPA |
| CLONE-26 | 9.06 | 13.1 | 93.2 | 98.28 | 1.72 | 1.62 | 98.09 | 0.28 |
| CLONE-24 | 7.92 | 13.5 | 93.0 | 98.75 | 1.25 | 1.39 | 98.39 | 0.21 |
| CLONE-10 | 7.60 | 14.9 | 86.1 | 98.72 | 1.28 | 1.28 | 98.47 | 0.24 |
| CLONE-17 | 6.72 | 22.7 | 88.9 | 98.82 | 1.18 | 1.83 | 97.99 | 0.18 |
| CLONE-18 | 6.54 | 11.2 | 74.7 | 98.72 | 1.28 | 1.46 | 98.33 | 0.21 |
| CLONE-13 | 6.48 | 15.8 | 81.6 | 98.40 | 1.60 | 1.35 | 98.45 | 0.20 |
| CLONE-06 | 6.18 | 11.8 | 82.6 | 98.81 | 1.19 | 1.06 | 98.74 | 0.20 |
| CLONE-08 | 5.86 | 12.5 | 91.5 | 98.74 | 1.26 | 1.36 | 98.42 | 0.22 |
| CLONE-05 | 5.62 | 17.5 | 91.4 | 98.68 | 1.32 | 1.61 | 98.18 | 0.21 |
| CLONE-19 | 3.68 | 3.2 | 95.9 | 98.39 | 1.61 | 1.79 | 97.98 | 0.23 |

HPLC = high performance liquid chromatography
FB D14 VCC = Fed-batch day 14 viable cell count
CPA = corrected peak area

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A modified, non-naturally occurring internal ribosome entry site (IRES) comprising or consisting of a polynucleotide sequence having at least 95%, 96%, 97%, 98% or 99% sequence identity, to SEQ ID NO: 1, and comprising one or both of:
i. 1-5 modifications (e.g., substitutions, additions or deletions) within positions 135-155, e.g., within positions 136-150; or
ii. 1-5 modifications (e.g., substitutions, additions or deletions) within positions 265-280, e.g., within positions 269-274, wherein the positions are with reference to SEQ ID NO: 1.

2. The modified IRES of claim 1, comprising or consisting of a polynucleotide sequence of any one of SEQ ID NOs: 3-4, 9-25 and 33-42 or a polynucleotide sequence comprising at least 95%, 96%, 97%, 98% or 99% sequence identity to any one of SEQ ID NOs: 3-4, 9-25 and 33-42.

3. The modified IRES of claim 1, wherein the modified IRES is no longer than 588 nucleotide bases in length.

4. An expression cassette comprising from 5′ to 3′ the following operably linked elements:
1) A promoter;
2) A polynucleotide encoding a protein of interest;
3) A modified IRES as set forth in claim 1;
4) A selection marker; and
5) A polyadenylation (polyA) signal sequence.

5. The expression cassette of claim 4, wherein the protein of interest is a therapeutic protein.

6. The expression cassette of claim 4, wherein the therapeutic protein is selected from an antibody and an immunoadhesin (e.g., an Fc fusion protein).

7. The expression cassette of claim 6, wherein the antibody is a multi-specific antibody.

8. The expression cassette of claim 4, wherein the promoter is a constitutive promoter.

9. The expression cassette of claim 4, wherein the promoter promotes or induces expression of the protein of interest in a mammalian host cell.

10. The expression cassette of claim 4, wherein the promoter is selected from a CMV promoter, a CAG promoter, an EF1a promoter, a PGK promoter, a SV40 promoter, an UBC promoter, and a beta-actin promoter.

11. The expression cassette of claim 4, wherein the modified IRES comprises a polynucleotide sequence of any one of SEQ ID NOs: 12-14, 17, 22, 24, 40 and 42.

12. The expression cassette of claim 4, wherein the selection marker comprises a polynucleotide encoding a glutamine synthetase (GS) or an antibiotic resistance protein.

13. The expression cassette of claim 12, wherein the antibiotic resistance protein is encoded by NeoR, BsdR, HygR, PuroR, BleoR (a.k.a., ZeoR).

14. The expression cassette of claim 12, wherein the GS is selected from a human GS, a fish GS, a koala GS, a hamster GS and variants thereof.

15. The expression cassette of claim 14, wherein the GS is encoded by a polynucleotide of any one of SEQ ID NOs: 26-30.

16-17. (canceled)

18. The expression cassette of claim 4, wherein the modified IRES comprises SEQ ID NO: 40, and the selection marker comprises a polynucleotide encoding a human GS or a hamster GS.

19. The expression cassette of claim 18, wherein the modified IRES comprises SEQ ID NO: 40, and the selection marker comprises a polynucleotide of SEQ ID NO: 28 or SEQ ID NO: 29.

20. The expression cassette of claim 4, wherein the modified IRES is selected from SEQ ID NOs: 11-25 and 42, and the selection marker comprises a polynucleotide encoding human variant GS-R324S (SEQ ID NO: 30).

21-22. (canceled)

23. The expression cassette of claim 4, wherein the modified IRES is selected from SEQ ID NOs: 11-25 and 42, and the selection marker comprises a polynucleotide encoding an antibiotic resistance gene, e.g., BleoR.

24-25. (canceled)

26. The expression cassette of claim 4, wherein the modified IRES comprises SEQ ID NO: 17, and the selection marker comprises a polynucleotide encoding an antibiotic resistance gene, e.g., BsdR or ZeoR.

27. The expression cassette of claim 4, wherein the polyA signal sequence is a SV40 polyA or a bovine growth hormone (BGH) polyA.

28. An expression cassette comprising from 5′ to 3′ the following operably linked elements:
1) A promoter;
2) A polynucleotide encoding an Fc fusion protein or an immunoadhesin;
3) A modified IRES comprising a polynucleotide of any one of SEQ ID NOs: 3-4, 9-25 and 33-42;
4) A selection marker encoding a human GS or a koala GS; and
5) A polyadenylation (polyA) signal sequence.

29-37. (canceled)

38. A vector comprising one or more expression cassettes of claim 4.

39-89. (canceled)

90. A host cell comprising the vector of claim 38.

91-99. (canceled)

100. A method of expressing a protein of interest, comprising:
a) Introducing into a mammalian host cell a vector of claim 38; and
b) Culturing the host cell under conditions to allow expression of the protein of interest.

101-103. (canceled)


SEQUENCE LISTING (42 sequences)

SEQ ID NO: 1 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 2 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaccggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 3 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctacccgtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 4 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctacccctctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 5 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctgcagacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 6 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctgaaaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 7 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctgttaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 8 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggagacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 9 - DNA, 556 bp, synthetic construct
  source 1..556: mol_type = other DNA
  variation 143: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 10 - DNA, 556 bp, synthetic construct
  source 1..556: mol_type = other DNA
  variation 144: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggttttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 11 - DNA, 555 bp, synthetic construct
  source 1..555: mol_type = other DNA
  variation 146..147: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 12 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttggcctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 13 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcggctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 14 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctcgcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 15 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccaccacgcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 16 - DNA, 556 bp, synthetic construct
  source 1..556: mol_type = other DNA
  variation 271: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctgcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 17 - DNA, 556 bp, synthetic construct
  source 1..556: mol_type = other DNA
  variation 273: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 18 - DNA, 556 bp, synthetic construct
  source 1..556: mol_type = other DNA
  variation 274: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgcaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 19 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcgtaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 20 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctacgggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 21 - DNA, 556 bp, synthetic construct
  source 1..556: mol_type = other DNA
  variation 145: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtcttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 22 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttggcctctcgccaaaggaatgcaaggtctgttgaatgtcgtgaaggaagcagttcctctggaagcttcttgaagacaaacaacgtctgtagcgaccctttgcaggcagcggaaccccccacctcgcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 23 - DNA, 556 bp, synthetic construct
  source 1..556: mol_type = other DNA
  variation 271: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttggcctctcgccaaaggaatgcaaggtctgttgaatgtcgtgaaggaagcagttcctctggaagcttcttgaagacaaacaacgtctgtagcgaccctttgcaggcagcggaaccccccacctgcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 24 - DNA, 555 bp, synthetic construct
  source 1..555: mol_type = other DNA
  variation 146..147: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctcccctctcgccaaaggaatgcaaggtctgttgaatgtcgtgaaggaagcagttcctctggaagcttcttgaagacaaacaacgtctgtagcgaccctttgcaggcagcggaaccccccacctcgcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 25 - DNA, 554 bp, synthetic construct
  source 1..554: mol_type = other DNA
  variation 146..147: note = deletion
  variation 271: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctcccctctcgccaaaggaatgcaaggtctgttgaatgtcgtgaaggaagcagttcctctggaagcttcttgaagacaaacaacgtctgtagcgaccctttgcaggcagcggaaccccccacctgcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 26 - DNA, 1119 bp, synthetic construct
  source 1..1119: mol_type = other DNA
atggctacctctgcctccagccacctgaacaagaacatcaagcagatgtacatgaacctgcctcagggcaacaaggtgctggccatgtatatctggatcgacggcaccggcgagggcctgagatgcaagacaagaaccctggactccgagcctaagtccatcgacgatctgcccgagtggaacttcgacggctcctccacctatcagtccgagggctccaactccgacatgtacttggtgcctgccgctctgttcagggaccctttcagaaaggaccccaacaagctggtgttctgcgaggtgttcaagtacaaccggaagcctgccgagacaaacctgcggcatacctgcaagcggatcatggacatggtcaccaaccagaaaccttggtttggcatggaacaagagtacaccctgatgggcaccgacggccatccttttggctggccttctaacggcttccctggacctcagggcccttactattgtggcgtgggcgccgataaggcctacggcagagatatcgctgaggcccactacagagcctgtctgtacgctggcgtgaagatcgtgggcaccaatgccgaggctatgccctctcagtgggagttccagatcggcccttgtgaaggcatcgagatgggcgatcatctgtgggtcgcccggttcatcctgcacagagtgtgcgaggacttcggcatgatcgtgaccttcgatcccaagcctattcctggcaactggaacggcgctggctgccacaccaacttctctaccaaggccatgcgggaagagaacggcctgaagttcatcgaggaagccatcgagcggctgtccaagcggcacaagtaccacatcagagcctacgatcccaaaggcggcctggacaatgctagaaggctgaccggcttcaacgagacatccaacatcaacgagttctctgccggcgtggccaacagaggcgccagcatcagaatccccagaatggtcggacaggacaagaagggctacttcgaggacagacggccttccgccaactgcgatccttacgctgtgacagaggccctgatccggacctgcctgttgaatgagacaggcgaggaacccttccagtacaagaac

SEQ ID NO: 27 - DNA, 1116 bp, synthetic construct
  source 1..1116: mol_type = other DNA
atggctacctccgcctcctccaagctgtccaaggctatgaagcagcagtacatggacctgcctcagggcgagaaggtgcaagtgatgtatatctggatcgacggcaccggcgagggcctgagatgcaagacaagaaccctggactccgagcctaagtccgtggaagaactgcccgagtggaacttcgacggctccagcacatatcaggccgagggctccaactccgacatgtatctgttccctcaagccatgttcagggaccctttccggaaggaccccaacaagctggtgctgtgcgacgtgctgaagtacaaccacaagcctgccgagacaaacctgcggcagtcctgcaagaaaatcatggacatggtgcagaatcagcacccttggtttggcatggaacaagagtacaccctgctgggcaccgacggccatccttttggctggccttctaacggcttccctggacctcagggcccttactattgtggcgtgggcgccgataaggcctacggcagagatgtggtggaagcccactacagagcctgtctgtacgctggcgtgaagatctgtggcaccaacgccgaagtgatgcccgctcagtgggagtttcaagtgggcccttgtgaaggcatcgagatgggcgatcatctgtgggtcgccagattcctgctgcacagagtgtgcgaggatttcggcgtggtggccagcttcgaccccaagcctattcctggcaactggaacggcgctggctgccacaccaacttctccaccaaagagatgcgcgaggaaggcggcctgaagcacatcgaggaatccatcgagaagctgggcaagagacaccagtaccacatccgggcctacgatcccaaaggcggactggataatgccagaaggctgaccggccaccatgagacatccaacatcaacgagttctctgccggcgtggccaacagaggcgccagcatcagaatccctagatccgtgggccaagagaagaagggctacttcgaggacagacggccttccgccaactgcgatccttacgctgtgaccgaggctctgatccggacctgtctgctgaacgaagagggcgaagtgcccgagaagtacatg

SEQ ID NO: 28 - DNA, 1119 bp, synthetic construct
  source 1..1119: mol_type = other DNA
atggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaac

SEQ ID NO: 29 - DNA, 1119 bp, synthetic construct
  source 1..1119: mol_type = other DNA
atgaccacctcagcaagttcccacttaaataaaggcatcaagcaggtgtacatgtccctgcctcagggtgagaaagtccaggccatgtatatctggatcgatggtactggagaaggactgcgctgcaagacccggaccctggacagtgagcccaagtgtgtggaagagttgcctgagtggaatttcgatggctctagtactttacagtctgagggttccaacagtgacatgtatctcgtgcctgctgccatgtttcgggaccccttccgtaaggaccctaacaagctggtgttatgtgaagttttcaagtacaatcgaaggcctgcagagaccaatttgaggcacacctgtaaacggataatggacatggtgagcaaccagcacccctggtttggcatggagcaggagtataccctcatggggacagatgggcacccctttggttggccttccaacggcttcccagggccccagggtccatattactgtggtgtgggagcagacagagcctatggcagggacatcgtggaggcccattaccgggcctgcttgtatgctggagtcaaaattgcggggactaatgccgaggtcatgcctgcccagtgggaatttcagattggaccttgtgaaggaatcagcatgggagatcatctctgggtggcccgtttcatcttgcatcgtgtgtgtgaagactttggagtgatagcaacctttgatcctaagcccattcctgggaactggaatggtgcaggctgccataccaacttcagcaccaaggccatgcgggaggagaatggtctgaagtacatcgaggaggccattgagaaactaagcaagcggcaccagtaccacatccgtgcctatgatcccaagggaggcctggacaatgcccgacgtctaactggattccatgaaacctccaacatcaacgacttttctgctggtgtagccaatcgtagcgccagcatacgcattccccggactgttggccaggagaagaagggttactttgaagatcgtcgcccctctgccaactgcgaccccttttcggtgacagaagccctcatccgcacgtgtcttctcaatgaaaccggcgatgagcccttccagtacaaaaat

SEQ ID NO: 30 - DNA, 1119 bp, synthetic construct
  source 1..1119: mol_type = other DNA
atgaccacctcagcaagttcccacttaaataaaggcatcaagcaggtgtacatgtccctgcctcagggtgagaaagtccaggccatgtatatctggatcgatggtactggagaaggactgcgctgcaagacccggaccctggacagtgagcccaagtgtgtggaagagttgcctgagtggaatttcgatggctctagtactttacagtctgagggttccaacagtgacatgtatctcgtgcctgctgccatgtttcgggaccccttccgtaaggaccctaacaagctggtgttatgtgaagttttcaagtacaatcgaaggcctgcagagaccaatttgaggcacacctgtaaacggataatggacatggtgagcaaccagcacccctggtttggcatggagcaggagtataccctcatggggacagatgggcacccctttggttggccttccaacggcttcccagggccccagggtccatattactgtggtgtgggagcagacagagcctatggcagggacatcgtggaggcccattaccgggcctgcttgtatgctggagtcaaaattgcggggactaatgccgaggtcatgcctgcccagtgggaatttcagattggaccttgtgaaggaatcagcatgggagatcatctctgggtggcccgtttcatcttgcatcgtgtgtgtgaagactttggagtgatagcaacctttgatcctaagcccattcctgggaactggaatggtgcaggctgccataccaacttcagcaccaaggccatgcgggaggagaatggtctgaagtacatcgaggaggccattgagaaactaagcaagcggcaccagtaccacatccgtgcctatgatcccaagggaggcctggacaatgcccgacgtctaactggattccatgaaacctccaacatcaacgacttttctgctggtgtagccaatcgtagcgccagcatatccattccccggactgttggccaggagaagaagggttactttgaagatcgtcgcccctctgccaactgcgaccccttttcggtgacagaagccctcatccgcacgtgtcttctcaatgaaaccggcgatgagcccttccagtacaaaaat

SEQ ID NO: 31 - skipped

SEQ ID NO: 32 - DNA, 850 bp, synthetic construct
  source 1..850: mol_type = other DNA
ttgaaagccgggggtgggagatccggattgccagtctgctcgatatcgcaggctgggtccgtgactacccactccccctttcaacgtgaaggctacgatagtgccagggcgggtactgccgtaagtgccaccccaaaataacaacagaccccccccccccccccccccccccccccccccccccccccccccccccccccccccccccccccccccccccccccccccccccccccccccccccccccccccccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgga

SEQ ID NO: 33 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctacgggtctttcccctctcgccaaaggaatgcaaggtctgttgaatgtcgtgaaggaagcagttcctctggaagcttcttgaagacaaacaacgtctgtagcgaccctttgcaggcagcggaaccccccacctcgcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 34 - DNA, 556 bp, synthetic construct
  source 1..556: mol_type = other DNA
  variation 143: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggctttcccctctcgccaaaggaatgcaaggtctgttgaatgtcgtgaaggaagcagttcctctggaagcttcttgaagacaaacaacgtctgtagcgaccctttgcaggcagcggaaccccccacctcgcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 35 - DNA, 556 bp, synthetic construct
  source 1..556: mol_type = other DNA
  variation 144: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggttttcccctctcgccaaaggaatgcaaggtctgttgaatgtcgtgaaggaagcagttcctctggaagcttcttgaagacaaacaacgtctgtagcgaccctttgcaggcagcggaaccccccacctcgcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 36 - DNA, 556 bp, synthetic construct
  source 1..556: mol_type = other DNA
  variation 147: note = deletion
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtcttcccctctcgccaaaggaatgcaaggtctgttgaatgtcgtgaaggaagcagttcctctggaagcttcttgaagacaaacaacgtctgtagcgaccctttgcaggcagcggaaccccccacctcgcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 37 - DNA, 555 bp, synthetic construct
  source 1..555: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 38 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttagcctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 39 - DNA, 557 bp, synthetic construct
  source 1..557: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttggcccctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 40 - DNA, 558 bp, synthetic construct
  source 1..558: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctgagcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 41 - DNA, 558 bp, synthetic construct
  source 1..558: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggacgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 42 - DNA, 558 bp, synthetic construct
  source 1..558: mol_type = other DNA
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat